(19) United States
(12) Patent Application Publication
(10) Pub. No.: US 2004/0202644 A1
(43) Pub. Date: Oct. 14, 2004

(54) Crystallization and structure of Staphylococcus aureus peptide deformylase

(76) Inventors: Eric T. Baldwin, Kalamazoo, MI (US); Melissa S. Harris, Marshall, MI (US)

Correspondence Address: PHARMACIA & UPJOHN, 301 HENRIETTA ST, 0228-32-LAW, KALAMAZOO, MI, 49007, US

(21) Appl. No.: 10/340,367

(22) Filed: Jan. 10, 2003

Related U.S. Application Data
(63) Continuation-in-part of application No. 09/896,580, filed on Jun. 29, 2001.
(60) Provisional application No. 60/215,550, filed on Jun. 30, 2000.

(51) Int. Cl. A61K 38/46; C07H 21/04; C12N 9/80; C12N 1/21; C12N 15/74

(52) U.S. Cl. 424/94.6; 435/69.1; 435/228; 435/252.3; 435/320.1; 536/23.2

(57) ABSTRACT

Staphylococcus aureus peptide deformylase has been crystallized, and the three-dimensional x-ray crystal structure has been solved to 1.9 Å resolution. The x-ray crystal structure is useful for solving the structure of other molecules or molecular complexes, and designing modifiers of peptide deformylase activity.

This application is a continuation in part of U.S. Ser. No. 09/896,580, filed on Jun. 29, 2001, which claims the benefit of the U.S. Provisional Application Serial No. 60/215,550, filed Jun. 30, 2000, both of which are incorporated herein by reference in their entirety.

### FIELD OF THE INVENTION

The present invention is related to the crystallization and structure determination of Staphylococcus aureus peptide deformylase (S. aureus pdf).

### BACKGROUND OF THE INVENTION

In all bacteria as well as mitochondria and chloroplasts the initiation of protein synthesis normally requires an N-formylated methionine residue. The special initiation tRNA, tRNA_{f}^{Met}, is charged with methionine by the Methionyl-tRNA synthetase (EC 6.1.10) which adds a methionine to either of the methionine tRNAs with the consumption of ATP. The formyl group is added to the charged tRNA_{f}^{Met} from 10-formyltetrahydrofolate which is catalyzed by methionine-tRNA_{f}^{Met} formyl-transferase (EC 2.1.2.9). The formylated tRNA is transferred to the ribosome where protein synthesis is initiated (FIG. 1). All nascent polypeptides are synthesized with N-formyl methionine at the n-terminus.

Mature proteins do not by and large retain n-formyl methionine at the n-terminus. In fact, a rather heterogenous population of amino acids are normally found at the n-terminus of mature proteins—alanine, glycine, serine, threonine, or methionine. Larger amino acids are rarely found, which suggests that multiple catabolic processing might occur after or in concert with protein synthesis. All known amino-terminal peptidases cannot use formylated peptides as substrates. After translation, the formyl group is removed by Peptide Deformylase (pdf) as illustrated in FIG. 2. This metalloenzyme (EC 3.5.1.27) removes the formyl group from the peptide amino-terminus and releases the protein for possible further processing by methionine aminopeptidase (MAP; EC 3.4.11.18). The formylation/deformylation cycle is unique to eubacteria and does not occur in eucaryotic protein synthesis. The essential deformylation activity of pdf makes it an attractive target for crystallization and structural studies. Such studies may lead to the design of new antibiotics.

### SUMMARY OF THE INVENTION

In one aspect, the present invention provides crystalline S. aureus peptide deformylase. Optionally, one or more methionine may be replaced with selenomethionine. The crystal may optionally include a coordinated metal ion selected from the group of metals consisting of Fe, Zn, Ni and combinations thereof.

In one embodiment, the crystal has the orthorhombic space group symmetry C222₁. Preferably, the unit cell has dimensions a, b, and c; wherein a is about 90 Å to about 100 Å, b is about 116 Å to about 128 Å, and c is about 45 Å to about 50 Å; and wherein α=β=γ=90°. More preferably, a is about 92 Å to about 95 Å, b is about 121 Å to about 124 Å, and c is about 47 Å to about 49 Å.

In another embodiment, the present invention provides a crystal of S. aureus peptide deformylase having the monoclinic space group symmetry C2. Preferably, the unit cell has dimensions a, b, and c; wherein a is about 85 Å to about 100 Å, b is about 35 Å to about 50 Å, and c is about 90 Å to about 110 Å; and wherein α=γ=90° and β is about 90° to about 95°. More preferably, a is about 91 Å to about 95 Å, b is about 41 Å to about 44 Å, and c is about 102 Å to about 105 Å.

In still another embodiment, the present invention provides a crystal of S. aureus peptide deformylase having the tetragonal space group symmetry P4₁, or P4₂2₁2. Preferably, the unit cell has dimensions a, b, and c; wherein a and b are about 130 Å to about 190 Å, and c is about 30 Å to about 70 Å; and wherein α=β=γ=90°. More preferably, a and b are about 160 Å to about 164 Å, and c is about 45 Å to about 49 Å.

In another aspect, the present invention provides a method for crystallizing an S. aureus peptide deformylase molecule or molecular complex. In one embodiment the method includes preparing a stock solution of purified S. aureus peptide deformylase at a concentration of about 1 mg/ml to about 50 mg/ml; contacting the stock solution with a precipitating solution containing about 1% by weight to about 35% by weight PEG having a number average molecular weight between about 300 and about 20,000; about 0 M to about 0.2 M MgCl₂; and about 0% by weight to about 25% by weight DMSO; the precipitating solution being buffered to a pH of about 5 to about 9; and allowing S. aureus peptide deformylase to crystallize from the resulting solution. Preferably, the precipitating solution contains about 15% by weight to about 25% by weight PEG having a number average molecular weight between about 3000 and about 5,000; about 0.05 M to about 0.15 M MgCl₂ and is buffered to a pH of about 8 to about 9.

In another embodiment the method for crystallizing an S. aureus peptide deformylase molecule or molecular complex includes preparing a stock solution of purified S. aureus peptide deformylase at a concentration of about 1 mg/ml to about 50 mg/ml; contacting the stock solution with a precipitating solution containing about 1% by weight to about 40% by weight PEG having a number average molecular weight between about 300 and about 20,000; about 0.005 M to about 0.5 M citric acid; about 0% by weight to about 25% by weight DMSO; and sufficient base to adjust the pH of the precipitating solution to about 5.0 to about 6.5; and allowing S. aureus peptide deformylase to crystallize from the resulting solution. Preferably, the precipitating solution contains about 1% by weight to about 40% by weight PEG having a number average molecular weight between about 2000 and about 4,000; about 0.05 M to about 0.2 M citric acid, and sufficient base to adjust the pH of the precipitating solution to about 5.0 to about 5.5.

In still another embodiment the method for crystallizing an S. aureus peptide deformylase molecule or molecular complex includes preparing a stock solution of purified S. aureus peptide deformylase at a concentration of about 1 mg/ml to about 50 mg/ml; contacting the stock solution with a precipitating solution containing about 0.2 M to about 1.5 M sodium citrate; about 0.005 M to about 0.5 M Hepes; about 0% by weight to about 25% by weight DMSO; and sufficient base to adjust the pH of the precipitating solution to about 7.0 to about 8.5; and allowing S. aureus peptide deformylase to crystallize from the resulting solution. Preferably, the precipitating solution contains about 25% by weight to about 35% by weight PEG having a number average molecular weight between about 2000 and about 4,000; about 0.05 M to about 0.2 M citric acid, and sufficient base to adjust the pH of the precipitating solution to about 5.0 to about 5.5.

In still another embodiment the method for crystallizing an S. aureus peptide deformylase molecule or molecular complex includes preparing a stock solution of purified S. aureus peptide deformylase at a concentration of about 1 mg/ml to about 50 mg/ml; contacting the stock solution with a precipitating solution containing about 1% by weight to about 40% by weight PEG having a number average molecular weight between about 300 and about 20,000; about 0 M to about 0.4 M MgCl₂; and about 0% by weight to about 25% by weight DMSO; the precipitating solution being buffered to a pH of about 7 to about 9; and allowing S. aureus peptide deformylase to crystallize from the resulting solution. Preferably, the precipitating solution contains about 15% by weight to about 35% by weight PEG having a number average molecular weight between about 3,000 and about 5,000; about 0.05 M to about 0.3 M MgCl₂; and the precipitating solution being buffered to a pH of about 8 to about 9.

In another aspect, the present invention provides a molecule or molecular complex including at least a portion of an S. aureus peptide deformylase or an S. aureus peptide deformylase-like active site including amino acids Gly58, Gly60, Leu61, Gln65, Glu109, Gly110, Cys111, Leu112, Ile150, His154, Glu155, and His158, the active site being defined by a set of points having a root mean square deviation of less than about 0.35 Å from points representing the backbone atoms of said amino acids as represented by structure coordinates listed in Table 1. Optionally, the molecule or molecular complex further includes a coordinated metal ion selected from the group of metals consisting of Fe, Zn, Ni and combinations thereof. Preferably, the metal ion is coordinated by the amino acids Cys111, His154, and His158.

In another aspect, the present invention provides a scalable three-dimensional configuration of points, at least a portion of said points, and preferably all of said points, derived from structure coordinates of at least a portion of an S. aureus peptide deformylase molecule or molecular complex listed in Table 1 and having a root mean square deviation of less than about 1.4 Å from said structure coordinates. Preferably, at least a portion of the points are derived from the S. aureus peptide deformylase structure coordinates are derived from structure coordinates representing the locations of at least the backbone atoms of a plurality of the amino acids defining at least one S. aureus peptide deformylase or S. aureus peptide deformylase-like active site, the active site including amino acids Gly58, Gly60, Leu61, Gln65, Glu109, Gly110, Cys111, Leu112, Ile150, His154, Glu155, and His158.

In another aspect, the present invention provides a machine-readable data storage medium including a data storage material encoded with machine readable data which, when using a machine programmed with instructions for using said data, displays a graphical three-dimensional representation of at least one molecule or molecular complex selected from the group consisting of (i) a molecule or molecular complex including at least a portion of an S. aureus peptide deformylase or an S. aureus peptide deformylase-like active site including amino acids Gly58, Gly60, Leu61, Gln65, Glu109, Gly110, Cys111, Leu112, Ile150, His154, Glu155, and His158, the active site being defined by a set of points having a root mean square deviation of less than about 0.35 Å from points representing the backbone atoms of said amino acids as represented by structure coordinates listed in Table 1.

In another aspect, the present invention provides a computer-assisted method for obtaining structural information about a molecule or a molecular complex of unknown structure including: crystallizing the molecule or molecular complex; generating an x-ray diffraction pattern from the crystallized molecule or molecular complex; applying at least a portion of the structure coordinates set forth in Table 1 to the x-ray diffraction pattern to generate a three-dimensional electron density map of at least a portion of the molecule or molecular complex whose structure is unknown.

In another aspect, the present invention provides a computer-assisted method for homology modeling an S. aureus peptide deformylase homolog including: aligning the amino acid sequence of an S. aureus peptide deformylase homolog with the amino acid sequence of S. aureus peptide deformylase SEQ ID NO:1 and incorporating the sequence of the S. aureus peptide deformylase homolog into a model of S. aureus peptide deformylase derived from structure coordinates set forth in Table 1 to yield a preliminary model of the S. aureus peptide deformylase homolog; subjecting the preliminary model to energy minimization to yield an energy minimized model; remodeling regions of the energy minimized model where stereochemistry restraints are violated to yield a final model of the S. aureus peptide deformylase homolog.

In another aspect, the present invention provides a computer-assisted method for identifying a potential modifier of S. aureus peptide deformylase activity including: supplying a computer modeling application with a set of structure coordinates of a molecule or molecular complex, the molecule or molecular complex including at least a portion of at least one S. aureus peptide deformylase or S. aureus peptide deformylase-like active site, the active site including amino acids Gly58, Gly60, Leu61, Gln65, Glu109, Gly110, Cys111, Leu112, Ile150, His154, Glu155, and His158; supplying the computer modeling application with a set of structure coordinates of a chemical entity; and determining whether the chemical entity is expected to bind to the molecule or molecular complex, wherein binding to the molecule or molecular complex is indicative of potential modification of S. aureus peptide deformylase activity.

In another aspect, the present invention provides a computer-assisted method for designing a potential modifier of S. aureus peptide deformylase activity including: supplying a computer modeling application with a set of structure coordinates of a molecule or molecular complex, the molecule or molecular complex including at least a portion of at least one S. aureus peptide deformylase or S. aureus peptide deformylase-like active site, the active site including amino acids Gly58, Gly60, Leu61, Gln65, Glu109, Gly110, Cys111, Leu112, Ile150, His154, Glu155, and His158; supplying the computer modeling application with a set of structure coordinates for a chemical entity; evaluating the potential binding interactions between the chemical entity and active site of the molecule or molecular complex; structurally modifying the chemical entity to yield a set of structure coordinates for a modified chemical entity; and determining whether the modified chemical entity is expected to bind to the molecule or molecular complex, wherein binding to the molecule or molecular complex is indicative of potential modification of S. aureus peptide deformylase activity.

In another aspect, the present invention provides a computer-assisted method for designing a potential modifier of S. aureus peptide deformylase activity de novo including: supplying a computer modeling application with a set of structure coordinates of a molecule or molecular complex, the molecule or molecular complex including at least a portion of at least one S. aureus peptide deformylase or S. aureus peptide deformylase-like active site, wherein the active site includes amino acids Gly58, Gly60, Leu61, Gln65, Glu109, Gly110, Cys111, Leu112, Ile150, His154, Glu155, and His158; forming a chemical entity represented by set of structure coordinates; and determining whether the chemical entity is expected to bind to the molecule or molecular complex, wherein binding to the molecule or molecular complex is indicative of potential modification of S. aureus peptide deformylase activity.

In another aspect, the present invention provides a method for making a potential modifier of S. aureus peptide deformylase activity, the method including chemically or enzymatically synthesizing a chemical entity to yield a potential modifier of S. aureus peptide deformylase activity, the chemical entity having been identified during a computer-assisted process including supplying a computer modeling application with a set of structure coordinates of a molecule or molecular complex, the molecule or molecular complex including at least a portion of a S. aureus peptide deformylase or S. aureus peptide deformylase-like active site; supplying the computer modeling application with a set of structure coordinates of a chemical entity; and determining whether the chemical entity is expected to bind to the molecule or molecular complex at the active site, wherein binding to the molecule or molecular complex is indicative of potential modification of S. aureus peptide deformylase activity.

In another aspect, the present invention provides a method for making a potential modifier of S. aureus peptide deformylase activity, the method including chemically or enzymatically synthesizing a chemical entity to yield a potential modifier of S. aureus peptide deformylase activity, the chemical entity having been designed during a computer-assisted process including supplying a computer modeling application with a set of structure coordinates of a molecule or molecular complex, the molecule or molecular complex including at least a portion of a S. aureus peptide deformylase or S. aureus peptide deformylase-like active site; supplying the computer modeling application with a set of structure coordinates for a chemical entity; evaluating the potential binding interactions between the chemical entity and the active site of the molecule or molecular complex; structurally modifying the chemical entity to yield a set of structure coordinates for a modified chemical entity; and determining whether the chemical entity is expected to bind to the molecule or molecular complex at the active site, wherein binding to the molecule or molecular complex is indicative of potential modification of S. aureus peptide deformylase activity.

In another aspect, the present invention provides a method for making a potential modifier of S. aureus peptide deformylase activity, the method including chemically or enzymatically synthesizing a chemical entity to yield a potential modifier of S. aureus peptide deformylase activity, the chemical entity having been designed during a computer-assisted process including supplying a computer modeling application with a set of structure coordinates of a molecule or molecular complex, the molecule or molecular complex including at least a portion of a S. aureus peptide deformylase or S. aureus peptide deformylase-like active site; forming a chemical entity represented by set of structure coordinates; and determining whether the chemical entity is expected to bind to the molecule or molecular complex at the active site, wherein binding to the molecule or molecular complex is indicative of potential modification of S. aureus peptide deformylase activity.

In still another aspect, the invention features a molecule or molecular complex comprising at least a portion of an S. aureus peptide deformylase or an S. aureus peptide deformylase-like active site comprising amino acids Arg56, Ser57, Gly58, Val59, Gly60, Leu61, Ala62, Leu105, Thr107, Gly108, Glu109, Gly110, Cys111, Leu112, Ser113, Ile150, Val151, His154, Glu155, and His158, the active site being defined by a set of points having a root mean square deviation of less than about 0.35 Å from points representing the backbone atoms of said amino acids as represented by structure coordinates listed in Table 1.

Table 1 lists the atomic structure coordinates for molecule Staphylococcus aureus peptide deformylase (S. aureus pdf) as derived by x-ray diffraction from a crystal of the protein. The following abbreviations are used in Table 1:

“Atom type” refers to the element whose coordinates are measured. The first letter in the column defines the element.

“X, Y, Z” crystallographically define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in all molecules of the crystal.

Abbreviations

The following abbreviations are used throughout this disclosure:

Staphylococcus aureus (S. aureus)

Escherichia coli (E. coli)

Haemophilis influenzae (Haemop. influenzae)

Bacillus subtilis (B. subtilis)

Mycoplasma pneumoniae (Mycopl. pneumoniae)

Peptide deformylase (pdf)

Isopropylthio-β-D-galactoside (IPTG)

(S)-2-O-(H-phosphonoxy)-L-caproyl-L-leucyl-p-nitroanilide (PCLNA)

Dimethyl sulfoxide (DMSO)

Polyethylene glycol (PEG)

Beta-mercaptoethanol (BME)

Optical density (OD)

Multiple anomalous dispersion (MAD)

Root mean square (r.m.s.)

Root mean square deviation (r.m.s.d.)

PNU-172550 is a compound having the following structure:

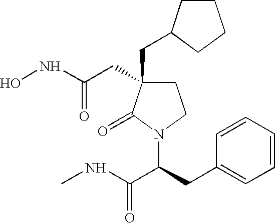

The following abbreviations are used for amino acids throughout this disclosure:

A = Ala = Alanine
V = Val = Valine
L = Leu = Leucine
I = Ile = Isoleucine
P = Pro = Proline
F = Phe = Phenylalanine
W = Trp = Tryptophan
M = Met = Methionine
G = Gly = Glycine
S = Ser = Serine
T = Thr = Threonine
C = Cys = Cysteine
Y = Tyr = Tyrosine
N = Asn = Asparagine
Q = Gln = Glutamine
D = Asp = Aspartic Acid
E = Glu = Glutamic Acid
K = Lys = Lysine
R = Arg = Arginine
H = His = Histidine

### BRIEF DESCRIPTION OF THE FIGURES

FIG. 1 is a schematic representation of the methionine cycle in bacteria.

FIG. 2 is a schematic representation of the reaction catalyzed by peptide deformylase.

FIG. 3 lists the amino acid sequences of peptide deformylases from various species of bacteria including Staphylococcus aureus peptide deformylase (pdf) with C-terminal 6×His tag (SEQ ID NO: 1); Escherichia coli pdf (SEQ ID NO:2); Haemophilis influenzae pdf (SEQ ID NO:3); Bacillus subtilis (SEQ ID NO:4); and Mycoplasma pneumoniae (SEQ ID NO:5); and Staphylococcus aureus def1 gene (a related but inactive form of the protein, also called Pseudo pdf) (SEQ ID NO:6). Alignments were generated from GCG SeqLab (Wisconsin Package Version 10.1, Genetics Computer Group, Madison, Wis.). The underlined residues show regions of importance to the activity of peptide deformylases. The highlighted amino acids show mutations for S. aureus pdf (SEQ ID NO:1).

FIG. 4 is a photograph illustrating 4-20% SDS PAGE gel of pseudo pdf, pdf1, and further purified pdf2.

FIG. 5 is a schematic secondary structure diagram of S. aureus pdf.

FIG. 6 is a depiction of the secondary structure of S. aureus peptide deformylase. The α-helices are starred and the β-sheets are not starred. Random coil connections are light gray. The single Zn/Fe atom is labeled **.

FIG. 7 is a stereo pair view of S. aureus peptide deformylase backbone from the same view as in FIG. 6.

FIG. 8 is a model showing the electrostatic surface potential for pdf. The positively charged region is indicated by the arrow (+100 kcal) while the negatively charged regions are gray (−100 kcal). The surface potential was created in MOSAIC2 (Computer Aided Drug Discovery) using point charge parameters derived from the AMBER force field (Weiner et al., J. Comput. Chem., 7:230-52 (1986)) and a formal charge of plus 2 for the metal ion.

FIG. 9 is a schematic model showing the active site metal ion (gray sphere). The metal ion may be Zn, Ni, or Fe. The ion is coordinated by protein sidechains H154, H158 and C111.

FIG. 10 is a sequence alignment based on x-ray structure comparisons for E. coli pdf and S. aureus pdf proteins.

FIG. 11 is a depiction of the secondary structure of pdf for a) S. aureus pdf and b) E. coli pdf. The n-terminus ends are starred.

FIG. 12 is a stereo pair view of the superimposed alpha carbons from S. aureus pdf (dark) and E. coli pdf (light). The metal ion is indicated by the sphere.

FIG. 13 is a stereo pair view of the superposition of the active site cavity of the E. coli pdf structure. Some selected residues from S. aureus pdf are labeled.

FIG. 14 a) is a schematic illustration of PCLNA inhibitor (Hao et al., Biochemistry, 38: 4712-19 (1999)) placing substituents into three pdf subsites. The S. aureus residue number is given first with the equivalent E. coli amino acid subsequent. The metal ion is the labeled sphere. FIG. 14 b) is a view of a surface rendering for the PCLNA complex with the E. coli enzyme with the location of the subsites indicated. The light gray surface represents hydrophobic surface associated with carbon atoms, dark gray for nitrogen atoms and medium gray for oxygen atoms.

FIG. 15 is a view of a model of the active site cleft of S. aureus pdf with PCLNA (from Hao et al., Biochemistry, 38: 4712-19 (1999)). The surface is colored according to atom type with all carbons in light gray, oxygens in medium gray, and nitrogens in dark gray. The six active site residues which are conserved between E. coli and S. aureus pdf are indicated in white. These residues line the bottom of the active site.

FIG. 16 is a view of a model of the surface rendering for PCLNA complex with E. coli enzyme (left) and of PCLNA with S. aureus enzyme (right). The light gray colors indicate the hydrophobic surface associated with carbon atoms, dark gray is for nitrogen atoms, and medium gray for oxygen atoms. Amino acid labeling indicates the surface corresponding to various residues.

FIG. 17 is a stereo view of the S1 subsite of pdf with PCLNA inhibitor. The amino acid sidechains which surround the P1, caproyl group, are indicated. Labels indicate the S. aureus amino acid first and the equivalent E. coli residue second. However, R97/N is indicated with the opposite nomenclature.

FIG. 18 is a stereo view of the S2 subsite of pdf with PCLNA inhibitor. The amino acid sidechains which surround the P2, leucyl group, are indicated. Labels indicate the S. aureus amino acid first and the equivalent E. coli residue second. However, R97/N is indicated with the opposite nomenclature.

FIG. 19 is a stereo view of the S3 subsite of pdf with PCLNA inhibitor. The amino acid sidechains which surround the P3, p-nitroanilide group, are indicated. Labels indicate the S. aureus amino acid first and the equivalent E. coli residue second.

FIG. 20 is model representation of hydrogen bonds made by Actinonin near the active site metal ion. The hydroxymate group makes hydrogen bonds with the backbone amide of Leucine-112, the side-chain acid of glutamic-155 and with the metal ion. The carbonyl of the P1′ alkyl residue makes a hydrogen bond with the backbone amide of Valine-59. S1′ indicates the hydrophobic pocket that binds the alkyl chain.

FIG. 21 is a model representation of the hydrogen bonds made by Actinonin in the S2′ and S3′ subsites. These include a hydrogen bond between the P1′ carbonyl and the backbone amide of V59 (see also FIG. 20), a poor hydrogen bond between the amide of S2′ and the carbonyl of G110, and a hydrogen bond between the S2′ carbonyl and the backbone amide of G110.

FIG. 22 is a model representation of the hydrogen bonds made by Thiorphan near the active site metal ion. The thiol group makes a hydrogen bond with the metal ion. The thiol group is nearly 3.4 Å from the backbone amide of Leucine-112 and is thus not a strong interaction. The carbonyl of the P1′ benzyl group residue makes a hydrogen bond with the backbone amide of Valine-59. S1′ indicates the hydrophobic pocket that binds the benzyl group.

FIG. 23 is a model representation of the hydrogen bonding interactions in the S1′ subsite. The thiol group is nearly 3.4 Å from the backbone amide of Leucine-112 and is thus not a strong interaction. The carbonyl of the P1′ benzyl group residue makes a hydrogen bond with the backbone amide of Valine-59. The S1′ hydrophobic pocket contains the benzyl group. The inhibitor amide does not make a hydrogen bond.

FIG. 24 is a model representation of the hydrogen bonding interactions in the S2′ subsite. The acid group of thiorphan makes a water mediated hydrogen bond with the backbone carbonyl of Serine-57 and with the sidechain of Arginine-56. The acid is also about 3.5-4.0 Å from Arginine-56 and may interact directly with a weak ionic bond. The carbonyl of the P1′ benzyl group residue makes a hydrogen bond with the backbone amide of Valine-59. The inhibitor amide does not make a hydrogen bond.

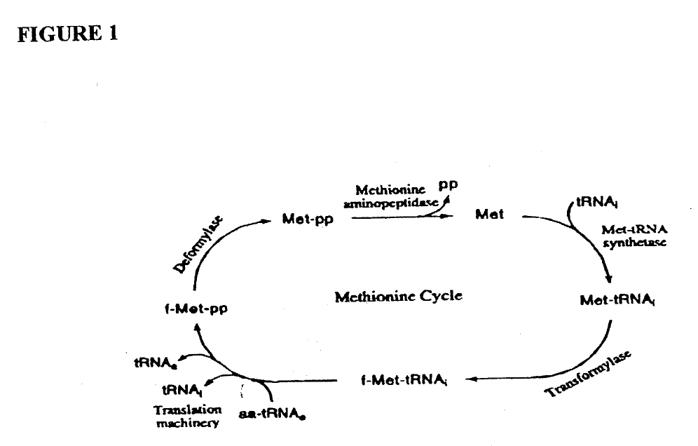

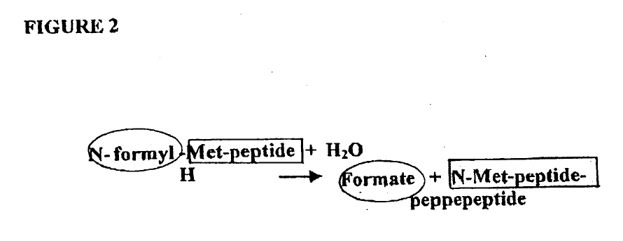

### DETAILED DESCRIPTION OF THE INVENTION

C222₁ Space Group Crystals

In one embodiment, crystals of S. aureus pdf have been obtained and belong to the C222₁ orthorhombic space group. Crystals were grown in four conditions, but crystals used for the structure solution were grown from 20% PEG 4000, 0.1M Tris pH=8.5 and 0.1M MgCl₂. Se-methionine pdf crystals were also grown and data was used to solve the pdf structure. Variation in buffer and buffer pH as well as other additives such as PEG is apparent to those skilled in the art and may result in similar crystals.

The S. aureus pdf protein was over-expressed and purified from E. coli. Crystallization attempts using pdf purified only by affinity Ni-NTA chromatography did not yield crystals, but the addition of an anion exchange purification step improved results. This further purified material resulted in many promising crystallization leads including four unique hits. Each of these hits were followed up using finely focused grid screens. All four conditions were pursued and characterized according to crystal behavior and quality. All small crystals were optimized though micro-seeding. Large, single crystals suitable for data collection were soaked in stabilization solution containing 25% glycerol prior to freezing for low temperature data collection. The useful crystals grown from the four diverse starting conditions all belong to the space group C222₁ with one molecule in the asymmetric unit. The unit cell parameters were a=94.1 b=121.87 c=47.58 Å.

Identical crystals of pdf were grown with Se-methionine pdf protein. One crystal was grown from 20% PEG 4000, 0.1M Tris pH=8.5 and 0.1m MgCl₂ and measured 0.22×0.22×0.6 micrometer. Data from this crystal was collected at the IMCA synchrotron facility and was found to belong to the space group C222₁ as well. The pdf structure was solved using this MAD data. However, the resulting structure could not be completely refined with the MAD data; so refinement was abandoned in favor of a new data set (see below).

A second crystal was grown in the presence of 2 mM of a potential inhibitor, 10% DMSO, 20% PEG 4000, 0.1M Tris pH=8.5 and 0.1M MgCl₂. This crystal measured 0.28×0.28×0.98 micrometer. No evidence for this compound was observed in the electron density map. After freezing the crystal, data was collected on a Siemens dual Hi-star. The crystal diffracted to 1.9 Å and molecular replacement was successfully performed using the MAD-derived model. This structure was refined to a final R-factor of 18.62%.

The orthorhombic crystal form could be prepared with or without compounds. The crystals belonging to the C222₁ space group generally have unit cell parameters with a 91.6 to 95.1 Å; b=121.3 to 123.5 Å; and c=47.6 to 48.4 Å. Crystals may be grown at 20° C., for example, by mixing a buffered protein sample with 19% PEG4000, 0.1M Tris pH 8.5 and 0.2M MgCl₂. Crystals may be stabilized in 25% PEG4000; 10% glycerol; 0.1M Tris pH 8.5 and 0.2M MgCl₂ for data collection.

C2 Space Group Crystals

The Monoclinic crystal form of S. aureus pdf, C2, with unit cell parameters ranging from a=90.8 to 95.1 Å; b=42.4 to 42.7 Å; and c=104.1 to 104.4 Å. Crystals were grown at 20° C. by mixing a buffered protein sample that included 5 mM PNU-172550 with an equal volume of 30% PEG 3000; 0.1M Na Citrate pH 5.2. Other compounds could be crystallized using the same procedure with variation in PEG concentration or pH. Crystals were stabilized in a solution containing PEG; Citrate, PNU-172550 and 10% glycerol for diffraction studies.

P4₂2₁2 Space Group Crystals

Another crystal form could also be prepared with PNU-172550. This tetragonal crystal form P4₂2₁2 has unit cell parameters ranging from a=b=160.4 to 163.5 Å and c=45.2 to 48.3 Å. Crystals are grown at 20° C. by mixing a buffered protein sample that included 5 mM PNU-172550 with an equal volume of 1.375 M Na Citrate and 0.1 M Na Hepes pH 7.5. Other compounds could be crystallized using the same procedure with variation in salts and buffers. Most often no stabilization solution was employed.

Comparison of S. aureus pdf and E. coli pdf Crystals

A number of structure determination reports have reported the crystallization of the pdf from E. coli as shown in Table 2. The present disclosure is believed to be the first crystallization of S. aureus pdf, and the reported crystal forms are also unique.

TABLE 2
The Space group and unit cell parameters for a variety of E. coli pdf
crystals.

| PDB No. | Space Group | A edge | B edge | C edge | Beta angle | A.U. |
|---|---|---|---|---|---|---|
| 1bs4 | C2₁ | 140.70 | 63.30 | 86.8 | 120.6 | 3 |
| 1bs5 | C2₁ | 143.4 | 64.10 | 84.6 | 123.2 | 3 |
| 1bs6 | C2₁ | 143.8 | 64.10 | 85.10 | 123.3 | 3 |
| 1bs7 | C2₁ | 143.4 | 64.0 | 84.50 | 123.0 | 3 |
| 1bs8 | C2₁ | 143.1 | 64.2 | 84.7 | 123.3 | 3 |
| 1bsz | C2₁ | 141.0 | 63.4 | 86.8 | 120.6 | 3 |
| 1dff | P6₁22 | 55.35 | 55.35 | 230.92 | 120 | 1 |
| 1icj | C2₁ | 140.7 | 63.4 | 86.9 | 120.6 | 3 |
| 1bsj | P6₅22 | 98.38 | 98.38 | 109.37 | 120 | 1 |
| 1bsk | P6₅22 | 100.11 | 100.11 | 111.34 | 120 | 1 |

X-Ray Crystallographic Analysis

Each of the constituent amino acids of S. aureus pdf is defined by a set of structure coordinates as set forth in Table 1. The term “structure coordinates” refers to Cartesian coordinates derived from mathematical equations related to the patterns obtained on diffraction of a monochromatic beam of x-rays by the atoms (scattering centers) of an S. aureus pdf complex in crystal form. The diffraction data are used to calculate an electron density map of the repeating unit of the crystal. The electron density maps are then used to establish the positions of the individual atoms of the S. aureus pdf protein or protein/ligand complex.

Slight variations in structure coordinates can be generated by mathematically manipulating the S. aureus pdf or S. aureus pdf/ligand structure coordinates. For example, the structure coordinates set forth in Table 1 could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of amino acids, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the S. aureus pdf or S. aureus pdf/ligand complex, as defined above, would not be expected to significantly alter the nature of ligands that could associate with the active sites. Thus, for example, a ligand that bound to the active site of S. aureus pdf would also be expected to bind to or interfere with another active site whose structure coordinates define a shape that falls within the acceptable error.

Binding Pockets/Active Sites/Other Structural Features

The present invention has provided, for the first time, information about the shape and structure of the active site of S. aureus pdf.

Active sites are of significant utility in fields such as drug discovery. The association of natural ligands or substrates with the active sites of their corresponding receptors or enzymes is the basis of many biological mechanisms of action. Similarly, many drugs exert their biological effects through association with the active sites of receptors and enzymes. Such associations may occur with all or any parts of the active site. An understanding of such associations helps lead to the design of drugs having more favorable associations with their target, and thus improved biological effects. Therefore, this information is valuable in designing potential modifiers of S. aureus pdf-like activity, as discussed in more detail below.

The term “active site (or binding pocket),” as used herein, refers to a region of a molecule or molecular complex, that, as a result of its shape, favorably associates with another chemical entity or compound. Thus, an active site may include or consist of features such as interfaces between domains. Chemical entities or compounds that may associate with an active site include, but are not limited to, cofactors, substrates, inhibitors, agonists, antagonists, etc.

The active site of S. aureus peptide deformylase may be represented by the amino acids in the following table, which are believed would fall within 5 Å of an incorporated modifier. Using structure coordinates of E. coli pdf with bound PCLNA and the present S. aureus pdf, the structures were superimposed using the Pharmacia program SUPERPDB.

In Model A, the 12 residues that are identical between E. coli pdf and S. aureus pdf were superimposed and chosen as the set to be minimized. The resulting distances between the α-Cs for the 12 residues, and the RMS for all the atoms in each of the corresponding residues were calculated and are reported in Table 3.

In Model B, the three residues which coordinate the metal atom (Cys111, His154, and His158 for S. aureus pdf) were chosen as the set to be minimized, and other residues within 2 Å were brought into the refinement. The resulting distances between the α-Cs for 18 active site amino acids and the RMS for all the atoms in each of the corresponding residues were calculated and are reported in Table 3.

In Model C, the 12 residues that are identical between E. coli pdf and S. aureus pdf were chosen as the set to be minimized, and other residues within 2 Å were brought into the refinement. The distances between the α-Cs for 18 active site amino acids and the RMS for all the atoms in each of the corresponding residues were calculated and are reported in Table 3.

TABLE 3
Active Site Residues

| | | Model A | | Model B | | Model C | |
|---|---|---|---|---|---|---|---|
| | | α-C | RMS (all | α-C | RMS (all | α-C | RMS (all |
| S. aureus | E. coli | Dist., Å | atoms) | Dist., Å | atoms) | Dist., Å | atoms) |
| Arg56 | Glu41 | — | — | Too Long | | Too Long | |
| Ser57 | Glu42 | — | — | 1.8337 | 2.1387 | 1.8194 | 2.1266 |
| Gly58 | Gly43 | 0.4093 | 0.4619 | 0.7992 | 0.8537 | 0.7834 | 0.8379 |
| Val59 | Ile44 | — | — | 0.4870 | 1.4130 | 0.4875 | 1.4151 |
| Gly60 | Gly45 | 0.5142 | 0.5109 | 0.4953 | 0.4869 | 0.4944 | 0.4876 |
| Leu61 | Leu46 | 0.4495 | 0.4802 | 0.4177 | 0.4842 | 0.4293 | 0.4902 |
| Gln65 | Gln50 | 0.1933 | 0.2696 | 0.4340 | 0.4239 | 0.4216 | 0.4156 |
| Leu105 | Ile86 | — | — | 0.9699 | 1.5517 | 0.9858 | 1.5510 |
| Pro106 | none | — | — | — | — | — | — |
| Thr107 | none | — | — | — | — | — | — |
| Gly108 | Glu87 | — | — | 1.7757 | 1.7415 | 1.7446 | 1.7150 |
| Glu109 | Glu88 | 0.2880 | 0.3478 | 0.6969 | 0.5832 | 0.6699 | 0.5618 |
| Gly110 | Gly89 | 0.2008 | 0.1993 | 0.6020 | 0.4590 | 0.5843 | 0.4439 |
| Cys111 | Cys90 | 0.3367 | 0.4177 | 0.3111 | 0.4429 | 0.3104 | 0.4398 |
| Leu112 | Leu91 | 0.6834 | 0.9241 | 0.6170 | 0.8266 | 0.6151 | 0.8243 |
| Asn117 | Arg97 | — | — | Too Long | | Too Long | |
| Tyr147 | Leu125 | — | — | 1.1351 | 1.1966 | 1.1011 | 1.1681 |
| Ile150 | Ile128 | 0.1519 | 0.3571 | 0.4175 | 0.5381 | 0.3877 | 0.5129 |
| Val151 | Cys129 | — | — | 0.5516 | 0.5981 | 0.5282 | 0.5765 |
| His154 | His132 | 0.2017 | 0.3312 | 0.2066 | 0.2882 | 0.2093 | 0.2909 |
| Glu155 | Glu133 | 0.2294 | 0.3944 | 0.3929 | 0.4743 | 0.3869 | 0.4745 |
| His158 | His136 | 0.2387 | 0.3944 | 0.2444 | 0.3116 | 0.2330 | 0.3050 |

| | | | | | | |
|---|---|---|---|---|---|---|
| Composite RMS | 0.36 | 0.46 | 0.83 | 0.98 | 0.81 | 0.97 |

The active site of S. aureus pdf preferably includes at least a portion of the amino acids Gly58, Gly60, Leu61, Gln65, Glu109, Gly110, Cys111, Leu112, Ile150, His154, Glu155, and His158; and more preferably at least a portion of the amino acids Arg56, Ser57, Gly58, Val59, Gly60, Leu61, Gln65, Leu105, Pro106, Thr107, Gly108, Glu109, Gly110, Cys111, Leu112, Asn117, Tyr147, Ile150, Val151, His154, Glu155, and His158, as shown in Table 1. As used herein, “at least a portion of the amino acids” means at least about 50% of the amino acids, preferably at least about 70% of the amino acids, more preferably at least about 90% of the amino acids, and most preferably all the amino acids. It will be readily apparent to those of skill in the art that the numbering of amino acids in other isoforms of S. aureus pdf may be different.

The amino acid constituents of an S. aureus pdf active site as defined herein, as well as selected constituent atoms thereof, are positioned in three dimensions in accordance with the structure coordinates listed in Table 1. In one aspect, the structure coordinates defining the active site of S. aureus pdf include structure coordinates of all atoms in the constituent amino acids; in another aspect, the structure coordinates of the active site include structure coordinates of just the backbone atoms of the constituent atoms.

The term “S. aureus pdf-like active site” refers to a portion of a molecule or molecular complex whose shape is sufficiently similar to at least a portion of the active site of S. aureus pdf as to be expected to bind related structural analogues. A structurally equivalent active site is defined by a root mean square deviation from the structure coordinates of the backbone atoms of the amino acids that make up the active sites in S. aureus pdf (as set forth in Table 1) of at most about 0.8 Å, and preferably less than about 0.35 Å. How this calculation is obtained is described below.

The term “associating with” refers to a condition of proximity between a chemical entity or compound, or portions thereof, and an S. aureus pdf molecule or portions thereof. The association may be non-covalent, wherein the juxtaposition is energetically favored by hydrogen bonding, van der Waals forces, or electrostatic interactions, or it may be covalent.

Accordingly, the invention thus provides molecules or molecular complexes including an S. aureus pdf active site or S. aureus pdf-like active site, as defined by the sets of structure coordinates described above.

The crystal structure of the Staphylococcus aureus peptide deformylase enzyme (the def2 gene product) has been determined by MAD phased X-ray crystallography to 2.0 Å resolution. The protein structure reveals a fold similar to but not identical to the well characterized E. coli enzyme. Differences also extend into the active site region and will play a role in the elaboration of peptide deformylase (pdf) specific inhibitors.

Description of the Structure of pdf

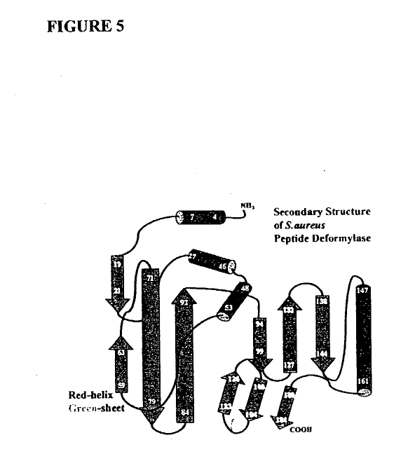

The pdf structure is composed mostly of β-sheet with two lengthy helical regions near the n and c-terminus (FIG. 5). The last helical region (147-161) forms the core of the structure and is also involved in catalysis. The β-sheet regions surround the centrally located, c-terminal helix and help to create the shallow cavity into which the substrates, formylated peptides, fit. The conserved motif HEXXH (H154 through H158) is found on this c-terminal helix and is involved in the coordination of the active site metal ion. Glutamic acid 155 is also likely essential for the catalytic process. Residues nearer the beginning of the helix are likely involved in specificity and are found near the opening of the cavity.

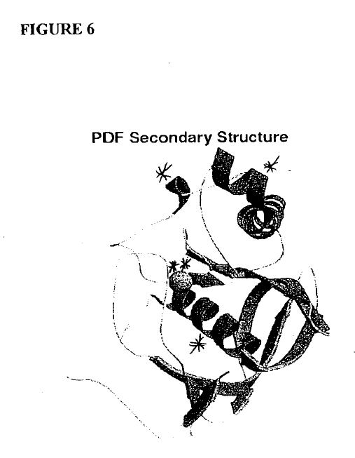

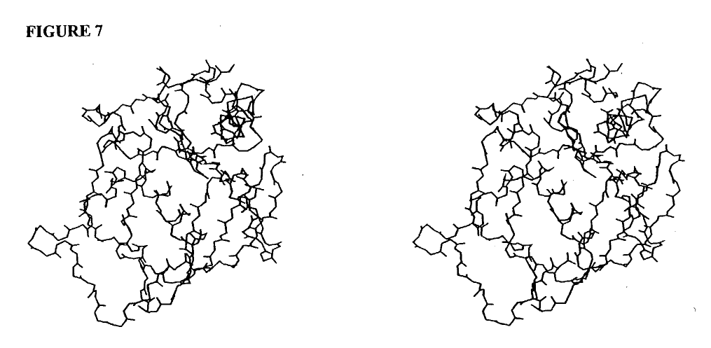

The n-terminal helical segments form a knot-like cluster on the “top” of the protein while the β-sheet regions are found on the lower half of the protein. A “thumb” region of coil extends from the lower sheet and covers the top of the metal ion (Center left FIG. 6). The β-sheet rich section is composed of three β-sheet elements, an n-terminal anti-parallel three stranded β-sheet, a central anti-parallel three stranded β-sheet and a c-terminal mixed β-sheet. The β-sheet elements pack around the active site helix and form the walls of the active site cavity. The c-terminus of the protein forms a last short strand of mixed β-sheet and is poised at the mouth of the active site (FIG. 7).

The structure has a large number of well ordered waters which have been placed into the electron density maps based upon 3 sigma difference density during the refinement as well as the potential for good hydrogen bonding. Many waters fill the active site cavity.

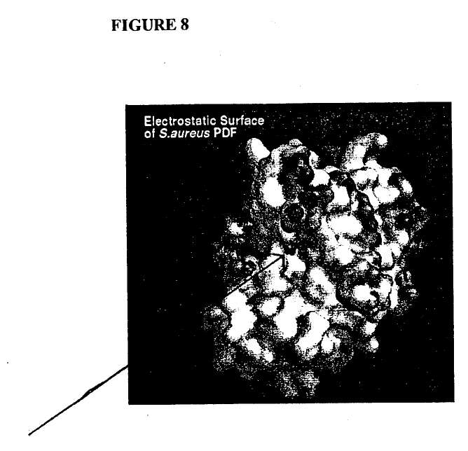

The electrostatic surface potential of pdf indicates an intense positively charged surface at the back of the active site cavity-due to the presence of the metal ion. The upper surface of the protein is richly decorated with negatively charged residues, while the lower surface is generally more neutral in potential (FIG. 8).

The Active Site Metal Ion

A large body of experimental data including X-ray and NMR structures suggests that pdf contains a metal ion in the active site (Meinnel et al., J. Bacteriol., 175:993-1000 (1993); Meinnel et al., J. Bacteriol., 177:1883-87 (1995); Chan et al., Biochemistry, 36:13904-09 (1997)). In addition, activity data (Rajagopalan et al., Biochemistry, 36:13910-18 (1997); Rajagopalan et al., J. Am. Chem. Soc., 119:12418-19 (1997)) point to iron as the most active metal ion. Data is consistent with this view; however, we have no experimental evidence based upon the present X-ray data to distinguish among ions like nickel, iron or zinc. From the initial MAD map it was clear that that a tetrahedrally coordinated metal ion is found in the three-dimensional structure of S. aureus pdf with water and the protein sidechains H154, H158, and CI111 coordinating the metal ion. The sequence motif HEXXH (Mazel et al., EMBO. J., 13:914-23 (1994)) in the c-terminal helix is a signature motif which is found in many metalloproteases including thermolysin (Blundell, Nat. Struct. Biol., 1:73-75 (1994); Jongeneel et al., FEBS Lett., 242: 211-14 (1989); Makarova et al., J. Mol. Biology, 292:11-17 (1999)). The glutamic acid residue of this motif probably plays a dual role in metal coordination and catalysis. The water molecule, which is a metal ligand, is tightly held in place by this glutamate residue in the present crystal structure. This residue likely plays a role in the protonation and deprotonation of reaction intermediates during the catalytic cycle in a manner similar to the role of the conserved glutamate in thermolysin (Matthews, Acc. Chem. Res., 21: 333-40 (1988); Chan et al., Biochemistry, 36:13904-09 (1997)).

Comparison of S. aureus pdf to E. coli Structure

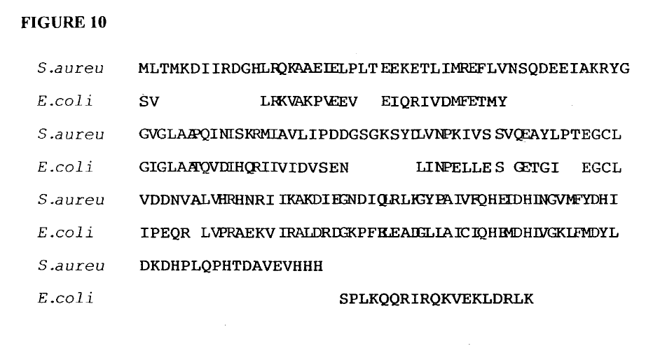

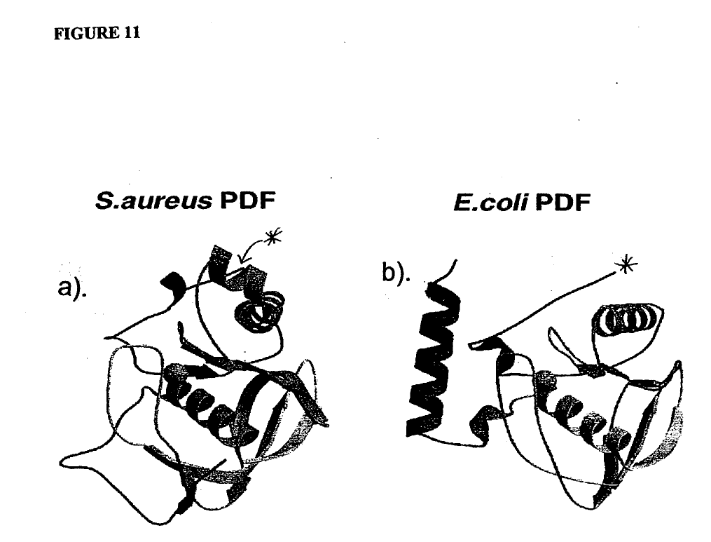

With the availability of numerous E. coli pdf X-ray and NMR structures (Table 1), it is possible to carry out a detailed comparison between these related enzymes. It should first be noted that the sequence identity between the E. coli and S. aureus enzymes is 45/134 or 33.5%. The rmsd for 134 α-carbons is 1.101 Å (1.457 Å for 861 common atoms; 1.189 Å for 536 main chain atoms). The vast majority of the identities (shown in FIG. 10) are limited to the conserved motifs (metal binding regions). A structure-based alignment of the protein sequence is given in FIG. 10. The poor sequence identity is not reflected in overall structural similarity. Both enzymes possess similar features in tertiary structure (FIG. 11).

S. aureus pdf has seven insertions with respect to the E. coli sequence (FIG. 10). The first insertion T3-M4 adds some additional hydrophobic surface area which forms a small surface for interaction with the third insertion (the extended n-terminal helix) N43-G54. The insertion after P25 adds one additional residue to the turn, which leads into the first long helix of pdf. This n-terminal helix is extended by an additional helix (insertion three N43-G54) which is not present in the E. coli structure. In the E. coli structure this helix is followed by a beta turn which drops down into the very conserved GXGLAA sequence which forms the third (and edge) strand of the n-terminal β-sheet. This strand also forms part of the wall of the active site crevice and provides loci for hydrogen bonding of peptide substrates (Hao et al., Biochemistry, 38: 4712-19 (1999)). The insertion of residues G81-G83 in the S. aureus structure extends the turn between strands II and III of the n-terminal β-sheet. The insertion of V100 is in the turn between strand I of the central anti-parallel β-sheet and the central strand of the c-terminal mixed sheet. Insertion six occurs at the end of the central strand of the mix sheet and includes P106 and T107. These residues are positioned at the opening of the active site crevice and may be important determinates of S. aureus specificity. The subsequent conserved residues EGCLS form the other wall of the active site crevice. Residue C111 at the center of this sequence is one of the active site metal ligands. The conserved glutamic acid projects downward to form a part of the crevice wall and makes a conserved salt bridge with R124, which is found in the center of the first strand of the mix β-sheet. The insertion of A119 results in a slight bulge of the connecting strand (with respect to the E. coli structure) which precedes the first strand of the c-terminal mixed β-sheet. This seventh insertion, the sixth insertion (P106/T107) [both located in the thumb] and the c-terminal extension are all in close proximity and constitute a S. aureus specific surface.

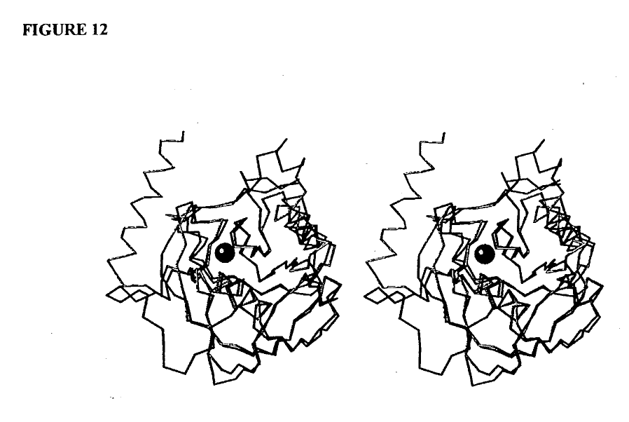

From the simplest comparison of these two X-ray structures one is immediately struck by the obvious difference at the c-terminus (FIG. 11). The E. coli enzyme has a long protruding α-helix which abutts the protein surface behind the active site cavity. The c-terminus of the S. aureus enzyme does not contain an equivalent α-helix, but wraps around the lower aspect of the thumb region to make a short stretch of β-sheet, terminating near the opening of the active site cavity. This is the major topological difference between the two structures, otherwise the proteins follow the same pattern and direction of secondary structure. Superposition of the two proteins permits a more detailed comparison of the alignment of secondary structural elements (FIG. 12) and was the basis of the structure-based sequence alignment of FIG. 10. A superficial evaluation would suggest that the core α-helix and the surrounding β-sheet is the most closely conserved region of the two proteins. Loops near the surface tend to be the location of insertions as is discussed above.

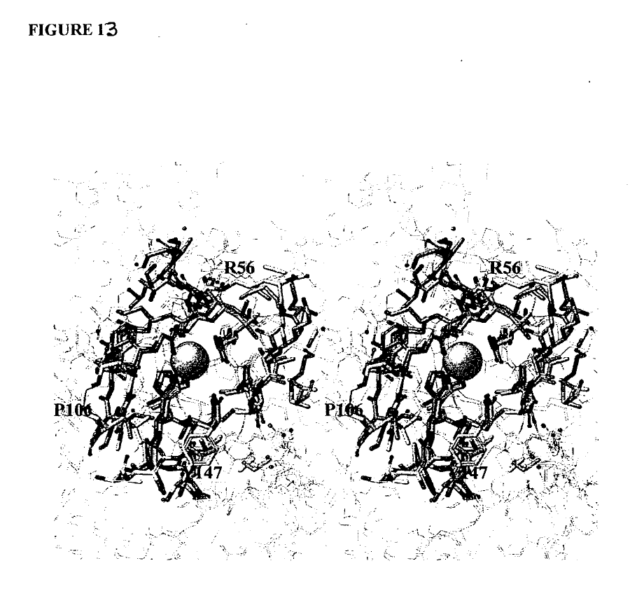

It follows from the low sequence identity between these two proteins, that the lining of the active site cavity would not be identical between S. aureus and E. coli. This expectation is in fact born out by the present structure (FIG. 13). Analysis of the active site cavity suggests that 9 residue changes are found in the crevice and the annulus about the crevice. These changes are indicated in the table below (Table 4). Some particularly interesting changes include the replacement of R56 (S. aureus) for R97 (E. coli) where the arginine sidechain is conserved but changes the side of the cavity from which it projects. A number of subtle hydrophobic-hydrophobic changes are observed as are a number of polar-polar changes.

TABLE 4
Difference in the active site residue between S. aureus and
E. coli pdf.

| S. aureus | E. coli | S. aureus | E. coli |
|---|---|---|---|
| V59 | I44 | T107 | E87 |
| S57 | E42 | P106 | — |
| R56 | E41 | L105 | I86 |
| N117 | R97 | Y147 | L125 |
| I150 | I128 | V151 | C129 |

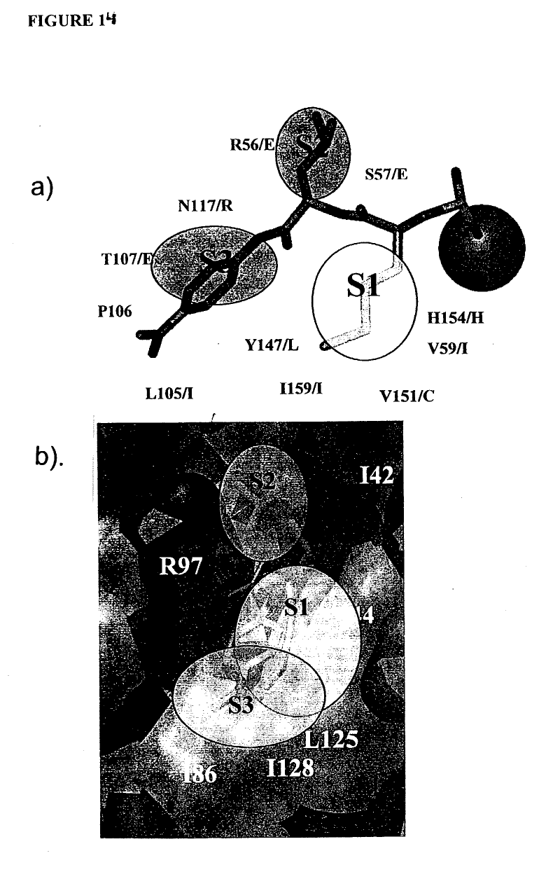

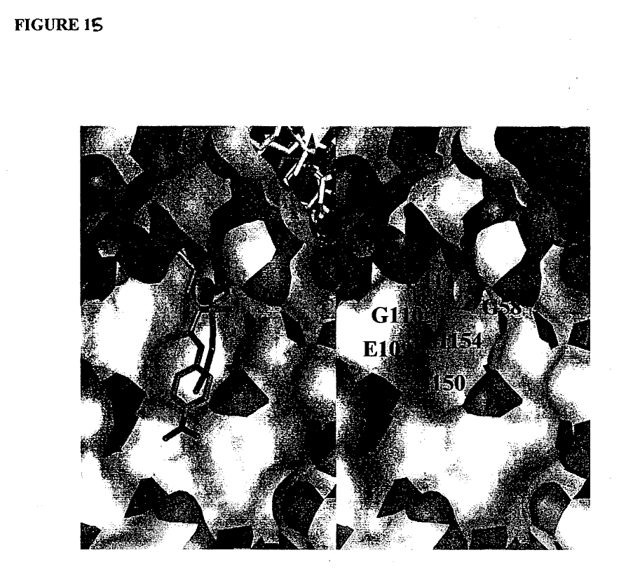

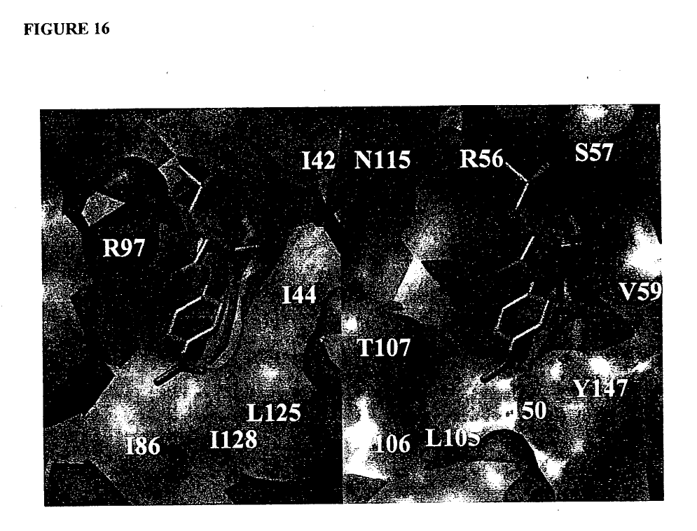

The X-ray structure of the (S)-2-O-(H-phosphonoxy)-L-caproyl-L-leucyl-p-nitroanilide (PCLNA) with the E. coli pdf enzyme (Hao et al., Biochemistry, 38: 4712-19 (1999)) can be used to guide the identification of the subsites (active sites) within the enzyme which accommodate the substrate amino acid sidechains (Schechter et al., BBRC, 27:157-62 (1967)). Using this scheme, the methionine analogue (caproyl), the P1 substituent, would occupy the S1 subsite; leucine, P2, the S2 subsite; and the p-nitroanilide, P3, the S3 subsite. With the PCLNA inhibitor as a frame of reference, superposition (as above) with the present S. aureus pdf X-ray structure permits the general comparison of the S. aureus with the corresponding E. coli subsites. This comparison is schematically shown in FIG. 14. The β-sheet mainchain conformation of the inhibitor forces the inhibitor substituents to adopt the typical down-up-down disposition observed for most peptidomimetic inhibitors. The P1 and P3 subsituents interact via the intra-molecular hydrophobic interface (between the caproyl and aromatic ring) to form a continuous surface which fills the S1 and S3 subsites. The P2 substituent projects away from the protein surface toward solvent.

Comparison of the E. coli and S. aureus crystal structures indicates that six residues in the region of the active site are conserved. In fact, five are always conserved in pdf sequences (ETB, data not shown). The residues come from the three regions of greatest sequence identity; Gxglaa, EGCls, and IxxqHexdhl, where the capitization indicates a conserved residue in the active site crevice. The first glycine is the lone invariant amino acid on the right side of the cleft (FIG. 15). The glutamic-glycine-cysteine triplet forms the invariant left side of the crevice. Finally, isoleucine and histidine are found at the bottom of the active site crevice (FIG. 15). These conserved residues form a continuous invariant surface which extends from the methionine (caproyl) site (S1) and up the left wall of the crevice. The variable residues encircle the upper aspect of the crevice. The differences account for the subtle differences in crevice shape when the two enzymes are compared—and presumably will be important determinates for inhibitor specificity.

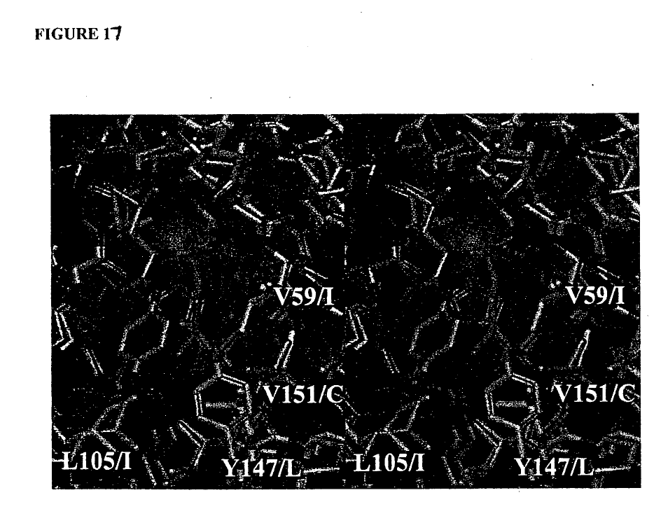

The S1 subsite has the greatest surface conservation between E. coli and S. aureus. This is due to the sequence conservation (outlined above) of the amino acids which form the bottom of the crevice—primarily H154, which also coordinates the metal ion, and I150. The long and fairly narrow hydrophobic subsite appears well-designed to cradle the preferred methionine residue. The rightside crevice wall is defined by V59(I, E. coli), Y147L, I150I, V151C, and L105I (FIG. 17). The subsite is an exclusive hydrophobic surface in E. coli; whereas, the hydroxyl group of Y147 introduces a potential hydrogen bonding group in the upper aspect of the rightside of the equivalent S. aureus subsite. The presence of the cysteine in the E. coli enzyme may contribute to the instability of the enzyme and may offer an advantage when working with S. aureus pdf.

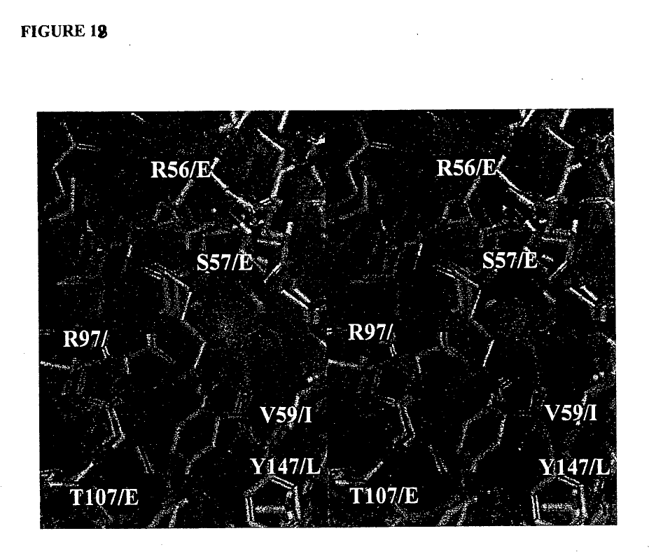

The S2 subsite is quite different between the two enzymes (FIG. 18). In E. coli R97 projects over the central leftside of the crevice and with E42 slightly narrows the entrance to the subsite. The principle hydrophobic interaction of the P2, leucyl, is with L91 (L112, in S. aureus). This residue is always hydrophobic, but not strictly conserved among pdf from different bacteria. The subsite continues unobstructed across the protein surface and is completely accessible to bulk solvent. In S. aureus pdf the E. coli R97 is lost and replaced with R56, which projects from the leftside of the crevice. Also, on the leftside the E. coli E42 is replaced with S57. The sidechain hydroxyl project directly into the S2 subsite and may provide a handle for P2 specific inhibitors directed towards S. aureus. Finally, the S2 subsite in S. aureus is obstructed by R56 which projects across the subsite limiting its depth, and concomitantly providing additional hydrogen bonding determinates.

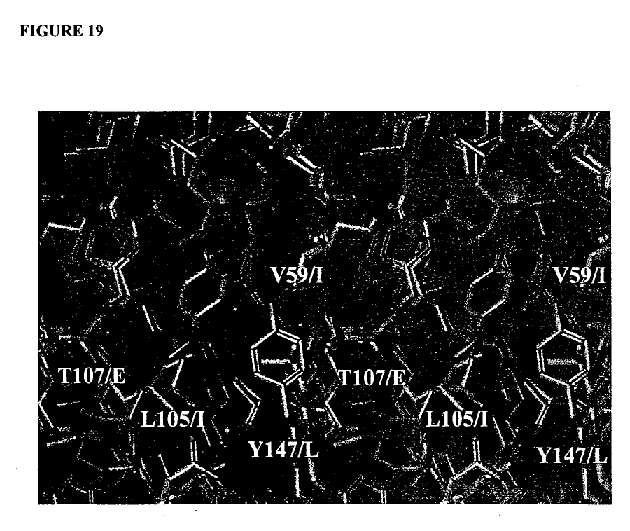

The S3 subsite is a broad somewhat flat hydrophobic surface in both enzymes (FIG. 19). Aside from an aliphatic contribution from E109, which is conserved among all pdf enzymes, there are no strictly conserved amino acids in the S3 subsite. The insertion of P106 broadens the subsite in the S. aureus species. The introduction of T107 for glutamatic acid is important as is the amino acid Y147 (as noted above). In the former case, the polar group projects into the subsite in the S. aureus protein and is available for a unique hydrogen bond. In addition the aromatic Y147 and the possible hydrogen bond from the hydroxyl differentiate the rightside of the S3 subsite. These differences between S. aureus and E. coli create distinct features for the S3 subsite, which may be exploited for bacteria-specific pdf inhibitors.

Three-Dimensional Configurations

The structure coordinates generated for S. aureus pdf or the S. aureus pdf/ligand complex or one of its active sites shown in Table 1 define a unique configuration of points in space. Those of skill in the art understand that a set of structure coordinates for protein or an protein/ligand complex, or a portion thereof, define a relative set of points that, in turn, define a configuration in three dimensions. A similar or identical configuration can be defined by an entirely different set of coordinates, provided the distances and angles between coordinates remain essentially the same. In addition, a scalable configuration of points can be defined by increasing or decreasing the distances between coordinates by a scalar factor while keeping the angles essentially the same.

The present invention thus includes the three-dimensional configuration of points derived from the structure coordinates of at least a portion of an S. aureus pdf molecule or molecular complex, as shown in Table 1, as well as structurally equivalent configurations, as described below. Preferably, the three-dimensional configuration includes points derived from structure coordinates representing the locations of a plurality of the amino acids defining the S. aureus pdf active site. In one embodiment, the three-dimensional configuration includes points derived from structure coordinates representing the locations the backbone atoms of a plurality of amino acids defining the S. aureus pdf active site, preferably Gly58, Gly60, Leu61, Gln65, Glu109, Gly110, Cys111, Leu112, Ile150, His154, Glu155, and His158; and more preferably Arg56, Ser57, Gly58, Val59, Gly60, Leu61, Gln65, Leu105, Pro106, Thr107, Gly108, Glu109, Gly110, Cys111, Leu112, Asn117, Tyr147, Ile150, Val151, His154, Glu155, and His158. In another embodiment, the three-dimensional configuration includes points derived from structure coordinates representing the locations of the side chain and the backbone atoms (other than hydrogens) of a plurality of the amino acids defining the S. aureus pdf active site, preferably Gly58, Gly60, Leu61, Gln65, Glu109, Gly110, Cys111, Leu112, Ile150, His154, Glu155, and His158; and more preferably Arg56, Ser57, Gly58, Val59, Gly60, Leu61, Gln65, Leu105, Pro106, Thr107, Gly108, Glu109, Gly110, Cys111, Leu112, Asn117, Tyr147, Ile150, Val151, His154, Glu155, and His158.

Likewise, the invention also includes the three-dimensional configuration of points derived from structure coordinates of molecules or molecular complexes that are structurally homologous to S. aureus pdf, as well as structurally equivalent configurations. Structurally homologous molecules or molecular complexes are defined below. Advantageously, structurally homologous molecules can be identified using the structure coordinates of S. aureus pdf (Table 1) according to a method of the invention.

The configurations of points in space derived from structure coordinates according to the invention can be visualized as, for example, a holographic image, a stereodiagram, a model or a computer-displayed image, and the invention thus includes such images, diagrams or models.

Structurally Equivalent Crystal Structures

Various computational analyses can be used to determine whether a molecule or the active site portion thereof is “structurally equivalent,” defined in terms of its three-dimensional structure, to all or part of S. aureus pdf or its active sites. Such analyses may be carried out in current software applications, such as the Molecular Similarity application of QUANTA (Molecular Simulations Inc., San Diego, Calif.) version 4.1, and as described in the accompanying User's Guide.

The Molecular Similarity application permits comparisons between different structures, different conformations of the same structure, and different parts of the same structure. The procedure used in Molecular Similarity to compare structures is divided into four steps: (1) load the structures to be compared; (2) define the atom equivalences in these structures; (3) perform a fitting operation; and (4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention equivalent atoms are defined as protein backbone atoms (N, Ca, C, and O) for all conserved residues between the two structures being compared. A conserved residue is defined as a residue that is structurally or functionally equivalent. Only rigid fitting operations are considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecule or molecular complex or active site thereof, or any portion thereof, that has a root mean square deviation of conserved residue backbone atoms (N, Ca, C, O) of less than about 1.4 Å, when superimposed on the relevant backbone atoms described by the reference structure coordinates listed in Table 1, is considered “structurally equivalent” to the reference molecule. That is to say, the crystal structures of those portions of the two molecules are substantially identical, within acceptable error. Particularly preferred structurally equivalent molecules or molecular complexes are those that are defined by the entire set of structure coordinates in Table 1, ±a root mean square deviation from the conserved backbone atoms of those amino acids of not more than 1.4 Å. More preferably, the root mean square deviation is less than about 0.8 Å, and preferably less than about 0.35 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a protein from the backbone of S. aureus pdf or an active site portion thereof, as defined by the structure coordinates of S. aureus pdf described herein.

Machine Readable Storage Media

Transformation of the structure coordinates for all or a portion of S. aureus pdf or the S. aureus pdf/ligand complex or one of its active sites, for structurally homologous molecules as defined below, or for the structural equivalents of any of these molecules or molecular complexes as defined above, into three-dimensional graphical representations of the molecule or complex can be conveniently achieved through the use of commercially-available software.

The invention thus further provides a machine-readable storage medium including a data storage material encoded with machine readable data which, when using a machine programmed with instructions for using said data, displays a graphical three-dimensional representation of any of the molecule or molecular complexes of this invention that have been described above. In a preferred embodiment, the machine-readable data storage medium includes a data storage material encoded with machine readable data which, when using a machine programmed with instructions for using said data, displays a graphical three-dimensional representation of a molecule or molecular complex including all or any parts of an S. aureus pdf active site or an S. aureus pdf-like active site, as defined above. In another preferred embodiment, the machine-readable data storage medium displays a graphical three-dimensional representation of a molecule or molecular complex defined by the structure coordinates of all of the amino acids in Table 1, ±a root mean square deviation from the backbone atoms of said amino acids of not more than 0.8 Å.

In an alternative embodiment, the machine-readable data storage medium includes a data storage material encoded with a first set of machine readable data which includes the Fourier transform of the structure coordinates set forth in Table 1, and which, when using a machine programmed with instructions for using said data, can be combined with a second set of machine readable data including the x-ray diffraction pattern of a molecule or molecular complex to determine at least a portion of the structure coordinates corresponding to the second set of machine readable data.

For example, a system for reading a data storage medium may include a computer including a central processing unit (“CPU”), a working memory which may be, e.g., RAM (random access memory) or “core” memory, mass storage memory (such as one or more disk drives or CD-ROM drives), one or more display devices (e.g., cathode-ray tube (“CRT”) displays, light emitting diode (“LED”) displays, liquid crystal displays (“LCDs”), electroluminescent displays, vacuum fluorescent displays, field emission displays (“FEDs”), plasma displays, projection panels, etc.), one or more user input devices (e.g., keyboards, microphones, mice, touch screens, etc.), one or more input lines, and one or more output lines, all of which are interconnected by a conventional bi-directional system bus. The system may be a stand-alone computer, or may be networked (e.g., through local area networks, wide area networks, intranets, extranets, or the internet) to other systems (e.g., computers, hosts, servers, etc.). The system may also include additional computer controlled devices such as consumer electronics and appliances.

Input hardware may be coupled to the computer by input lines and may be implemented in a variety of ways. Machine-readable data of this invention may be inputted via the use of a modem or modems connected by a telephone line or dedicated data line. Alternatively or additionally, the input hardware may include CD-ROM drives or disk drives. In conjunction with a display terminal, a keyboard may also be used as an input device.

Output hardware may be coupled to the computer by output lines and may similarly be implemented by conventional devices. By way of example, the output hardware may include a display device for displaying a graphical representation of an active site of this invention using a program such as QUANTA as described herein. Output hardware might also include a printer, so that hard copy output may be produced, or a disk drive, to store system output for later use.

In operation, a CPU coordinates the use of the various input and output devices, coordinates data accesses from mass storage devices, accesses to and from working memory, and determines the sequence of data processing steps. A number of programs may be used to process the machine-readable data of this invention. Such programs are discussed in reference to the computational methods of drug discovery as described herein. References to components of the hardware system are included as appropriate throughout the following description of the data storage medium.

Machine-readable storage devices useful in the present invention include, but are not limited to, magnetic devices, electrical devices, optical devices, and combinations thereof. Examples of such data storage devices include, but are not limited to, hard disk devices, CD devices, digital video disk devices, floppy disk devices, removable hard disk devices, magneto-optic disk devices, magnetic tape devices, flash memory devices, bubble memory devices, holographic storage devices, and any other mass storage peripheral device. It should be understood that these storage devices include necessary hardware (e.g., drives, controllers, power supplies, etc.) as well as any necessary media (e.g., disks, flash cards, etc.) to enable the storage of data.

Structurally Homologous Molecules, Molecular Complexes, and Crystal Structures

The structure coordinates set forth in Table 1 can be used to aid in obtaining structural information about another crystallized molecule or molecular complex. A “molecular complex” means a protein in covalent or non-covalent association with a chemical entity or compound. The method of the invention allows determination of at least a portion of the three-dimensional structure of molecules or molecular complexes which contain one or more structural features that are similar to structural features of S. aureus pdf. These molecules are referred to herein as “structurally homologous” to S. aureus pdf. Similar structural features can include, for example, regions of amino acid identity, conserved active site or binding site motifs, and similarly arranged secondary structural elements (e.g., α helices and β sheets). Optionally, structural homology is determined by aligning the residues of the two amino acid sequences to optimize the number of identical amino acids along the lengths of their sequences; gaps in either or both sequences are permitted in making the alignment in order to optimize the number of identical amino acids, although the amino acids in each sequence must nonetheless remain in their proper order. Preferably, two amino acid sequences are compared using the Blastp program, version 2.0.9, of the BLAST 2 search algorithm, as described by Tatusova et al., FEMS Microbiol. Lett., 174:247-50 (1999), and available at http://www.ncbi.nlm.nih.gov/gorf/b12.html. Preferably, the default values for all BLAST 2 search parameters are used, including matrix=BLOSUM62; open gap penalty=11, extension gap penalty=1, gap x_dropoff=50, expect=10, wordsize=3, and filter on. In the comparison of two amino acid sequences using the BLAST search algorithm, structural similarity is referred to as “identity.” Preferably, a structurally homologous molecule is a protein that has an amino acid sequence sharing at least 65% identity with the amino acid sequence of S. aureus pdf (SEQ ID NO: 1). More preferably, a protein that is structurally homologous to S. aureus pdf includes at least one contiguous stretch of at least 50 amino acids that shares at least 80% amino acid sequence identity with the analogous portion of S. aureus pdf. Methods for generating structural information about the structurally homologous molecule or molecular complex are well-known and include, for example, molecular replacement techniques.

Therefore, in another embodiment this invention provides a method of utilizing molecular replacement to obtain structural information about a molecule or molecular complex whose structure is unknown including the steps of:

(a) crystallizing the molecule or molecular complex of unknown structure;

(b) generating an x-ray diffraction pattern from said crystallized molecule or molecular complex; and

(c) applying at least a portion of the structure coordinates set forth in Table 1 to the x-ray diffraction pattern to generate a three-dimensional electron density map of the molecule or molecular complex whose structure is unknown.

By using molecular replacement, all or part of the structure coordinates of S. aureus pdf or the S. aureus pdf/ligand complex as provided by this invention (and set forth in Table 1) can be used to determine the structure of a crystallized molecule or molecular complex whose structure is unknown more quickly and efficiently than attempting to determine such information abinitio.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a structurally homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of S. aureus pdf or the S. aureus pdf/ligand complex according to Table 1 within the unit cell of the crystal of the unknown molecule or molecular complex so as best to account for the observed x-ray diffraction pattern of the crystal of the molecule or molecular complex whose structure is unknown. Phases can then be calculated from this model and combined with the observed x-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions,” in Meth. Enzymol., 115:55-77 (1985); M. G. Rossman, ed., “The Molecular Replacement Method,” Int. Sci. Rev. Ser., No. 13, Gordon & Breach, New York (1972)).

Structural information about a portion of any crystallized molecule or molecular complex that is sufficiently structurally homologous to a portion of S. aureus pdf can be resolved by this method. In addition to a molecule that shares one or more structural features with S. aureus pdf as described above, a molecule that has similar bioactivity, such as the same catalytic activity, substrate specificity or ligand binding activity as S. aureus pdf, may also be sufficiently structurally homologous to S. aureus pdf to permit use of the structure coordinates of S. aureus pdf to solve its crystal structure.

In a preferred embodiment, the method of molecular replacement is utilized to obtain structural information about a molecule or molecular complex, wherein the molecule or molecular complex includes at least one S. aureus pdf subunit or homolog. A “subunit” of S. aureus pdf is an S. aureus pdf molecule that has been truncated at the N-terminus or the C-terminus, or both. In the context of the present invention, a “homolog” of S. aureus pdf is a protein that contains one or more amino acid substitutions, deletions, additions, or rearrangements with respect to the amino acid sequence of S. aureus pdf, but that, when folded into its native conformation, exhibits or is reasonably expected to exhibit at least a portion of the tertiary (three-dimensional) structure of S. aureus pdf. For example, structurally homologous molecules can contain deletions or additions of one or more contiguous or noncontiguous amino acids, such as a loop or a domain. Structurally homologous molecules also include “modified” S. aureus pdf molecules that have been chemically or enzymatically derivatized at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

A heavy atom derivative of S. aureus pdf is also included as an S. aureus pdf homolog. The term “heavy atom derivative” refers to derivatives of S. aureus pdf produced by chemically modifying a crystal of S. aureus pdf. In practice, a crystal is soaked in a solution containing heavy metal atom salts, or organometallic compounds, e.g., lead chloride, gold thiomalate, thiomersal or uranyl acetate, which can diffuse through the crystal and bind to the surface of the protein. The location(s) of the bound heavy metal atom(s) can be determined by x-ray diffraction analysis of the soaked crystal. This information, in turn, is used to generate the phase information used to construct three-dimensional structure of the protein (T. L. Blundell and N. L. Johnson, Protein Crystallography, Academic Press (1976)).

Because S. aureus pdf can crystallize in more than one crystal form, the structure coordinates of S. aureus pdf as provided by this invention are particularly useful in solving the structure of other crystal forms of S. aureus pdf or S. aureus pdf complexes.

The structure coordinates of S. aureus pdf in Table 1 are also particularly useful to solve the structure of crystals of S. aureus pdf, S. aureus pdf mutants or S. aureus pdf homologs co-complexed with a variety of chemical entities. This approach enables the determination of the optimal sites for interaction between chemical entities, including candidate S. aureus pdf modifiers and S. aureus pdf. Potential sites for modification within the various binding site of the molecule can also be identified. This information provides an additional tool for determining the most efficient binding interactions, for example, increased hydrophobic interactions, between S. aureus pdf and a chemical entity. For example, high resolution x-ray diffraction data collected from crystals exposed to different types of solvent allows the determination of where each type of solvent molecule resides. Small molecules that bind tightly to those sites can then be designed and synthesized and tested for their potential modification of S. aureus pdf.

All of the complexes referred to above may be studied using well-known x-ray diffraction techniques and may be refined versus x-ray data to an R value of about 0.20 or less using computer software, such as X-PLOR (Yale University, (1992), distributed by Molecular Simulations, Inc.; see, e.g., Blundell & Johnson, supra; Meth. Enzymol., Vol. 114 & 115, H. W. Wyckoff et al., eds., Academic Press (985)). This information may thus be used to optimize known modifiers of S. aureus pdf activity, and more importantly, to design new modifiers of S. aureus pdf activity.

The invention also includes the unique three-dimensional configuration defined by a set of points defined by the structure coordinates for a molecule or molecular complex structurally homologous to S. aureus pdf as determined using the method of the present invention, structurally equivalent configurations, and magnetic storage media including such set of structure coordinates.

Further, the invention includes structurally homologous molecules as identified using the method of the invention.

Homology Modeling

Using homology modeling, a computer model of an S. aureus pdf homolog can be built or refined without crystallizing the homolog. First, a preliminary model of the S. aureus pdf homolog is created by sequence alignment with S. aureus pdf, secondary structure prediction, the screening of structural libraries, or any combination of those techniques. Computational software may be used to carry out the sequence alignments and the secondary structure predictions. Structural incoherences, e.g., structural fragments around insertions and deletions, can be modeled by screening a structural library for peptides of the desired length and with a suitable conformation. For prediction of the side chain conformation, a side chain rotamer library may be employed. Where the S. aureus pdf homolog has been crystallized, the final homology model can be used to solve the crystal structure of the homolog by molecular replacement, as described above. Next, the preliminary model is subjected to energy minimization to yield an energy minimized model. The energy minimized model may contain regions where stereochemistry restraints are violated, in which case such regions are remodeled to obtain a final homology model. The homology model is positioned according to the results of molecular replacement, and subjected to further refinement including molecular dynamics calculations.

Rational Drug Design

Computational techniques can be used to screen, identify, select and design chemical entities capable of associating with S. aureus pdf or structurally homologous molecules. Knowledge of the structure coordinates for S. aureus pdf permits the design and/or identification of synthetic compounds and/or other molecules which have a shape complementary to the conformation of the S. aureus pdf binding site. In particular, computational techniques can be used to identify or design chemical entities that are potential modifiers of S. aureus pdf activity, such as inhibitors, agonists and antagonists, that associate with an S. aureus pdf active site or an S. aureus pdf-like active site. Potential modifiers may bind to or interfere with all or a portion of the active site of S. aureus pdf, and can be competitive, non-competitive, or uncompetitive inhibitors; or interfere with dimerization by binding at the interface between the two monomers. Once identified and screened for biological activity, these inhibitors/agonists/antagonists may be used therapeutically or prophylactically to block S. aureus pdf activity and, thus, block bacterial growth. Structure-activity data for analogs of ligands that bind to or interfere with S. aureus pdf or S. aureus pdf-like active sites can also be obtained computationally.

The term “chemical entity,” as used herein, refers to chemical compounds, complexes of two or more chemical compounds, and fragments of such compounds or complexes. Chemical entities that are determined to associate with S. aureus pdf are potential drug candidates. Data stored in a machine-readable storage medium that displays a graphical three-dimensional representation of the structure of S. aureus pdf or a structurally homologous molecule, as identified herein, or portions thereof may thus be advantageously used for drug discovery. The structure coordinates of the chemical entity are used to generate a three-dimensional image that can be computationally fit to the three-dimensional image of S. aureus pdf or a structurally homologous molecule. The three-dimensional molecular structure encoded by the data in the data storage medium can then be computationally evaluated for its ability to associate with chemical entities. When the molecular structures encoded by the data is displayed in a graphical three-dimensional representation on a computer screen, the protein structure can also be visually inspected for potential association with chemical entities.

One embodiment of the method of drug design involves evaluating the potential association of a known chemical entity with S. aureus pdf or a structurally homologous molecule, particularly with an S. aureus pdf active site or S. aureus pdf-like active site. The method of drug design thus includes computationally evaluating the potential of a selected chemical entity to associate with any of the molecules or molecular complexes set forth above. This method includes the steps of: (a) employing computational means to perform a fitting operation between the selected chemical entity and a active site of the molecule or molecular complex; and (b) analyzing the results of said fitting operation to quantify the association between the chemical entity and the active site.

In another embodiment, the method of drug design involves computer-assisted design of chemical entities that associate with S. aureus pdf, its homologs, or portions thereof. Chemical entities can be designed in a step-wise fashion, one fragment at a time, or may be designed as a whole or “de novo.”

To be a viable drug candidate, the chemical entity identified or designed according to the method must be capable of structurally associating with at least part of an S. aureus pdf or S. aureus pdf-like active sites, and must be able, sterically and energetically, to assume a conformation that allows it to associate with the S. aureus pdf or S. aureus pdf-like active site. Non-covalent molecular interactions important in this association include hydrogen bonding, van der Waals interactions, hydrophobic interactions, and electrostatic interactions. Conformational considerations include the overall three-dimensional structure and orientation of the chemical entity in relation to the active site, and the spacing between various functional groups of an entity that directly interact with the S. aureus pdf-like active site or homologs thereof.

Optionally, the potential binding of a chemical entity to an S. aureus pdf or S. aureus pdf-like active site is analyzed using computer modeling techniques prior to the actual synthesis and testing of the chemical entity. If these computational experiments suggest insufficient interaction and association between it and the S. aureus pdf or S. aureus pdf-like active site, testing of the entity is obviated. However, if computer modeling indicates a strong interaction, the molecule may then be synthesized and tested for its ability to bind to or interfere with an S. aureus pdf or S. aureus pdf-like active site. Binding assays to determine if a compound actually binds to S. aureus pdf can also be performed and are well known in the art. Binding assays may employ kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof.

One skilled in the art may use one of several methods to screen chemical entities or fragments for their ability to associate with an S. aureus pdf or S. aureus pdf-like active site. This process may begin by visual inspection of, for example, an S. aureus pdf or S. aureus pdf-like active site on the computer screen based on the S. aureus pdf structure coordinates in Table 1 or other coordinates which define a similar shape generated from the machine-readable storage medium. Selected fragments or chemical entities may then be positioned in a variety of orientations, or docked, within the active site. Docking may be accomplished using software such as QUANTA and SYBYL, followed by energy minimization and molecular dynamics with standard molecular mechanics forcefields, such as CHARMM and AMBER.

Specialized computer programs may also assist in the process of selecting fragments or chemical entities. Examples include GRID (P. J. Goodford, J. Med. Chem., 28:849-57 (1985); available from Oxford University, Oxford, UK); MCSS (A. Miranker et al., Proteins: Struct. Funct. Gen., 11:29-34 (1991); available from Molecular Simulations, San Diego, Calif.); AUTODOCK (D. S. Goodsell et al., Proteins: Struct. Funct. Genet., 8:195-202 (1990); available from Scripps Research Institute, La Jolla, Calif.); and DOCK (I. D. Kuntz et al., J. Mol. Biol., 161:269-88 (1982); available from University of California, San Francisco, Calif.).

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of S. aureus pdf. This would be followed by manual model building using software such as QUANTA or SYBYL (Tripos Associates, St. Louis, Mo.).

Useful programs to aid one of skill in the art in connecting the individual chemical entities or fragments include, without limitation, CAVEAT (P. A. Bartlett et al., in Molecular Recognition in Chemical and Biological Problems, Special Publ., Royal Chem. Soc., 78:182-96 (1989); G. Lauri et al., J. Comput. Aided Mol. Des., 8:51-66 (1994); available from the University of California, Berkeley, Calif.); 3D database systems such as ISIS (available from MDL Information Systems, San Leandro, Calif.; reviewed in Y. C. Martin, J. Med. Chem. 35:2145-54 (1992)); and HOOK (M. B. Eisen et al., Proteins: Struc., Funct., Genet., 19:199-221 (1994); available from Molecular Simulations, San Diego, Calif.).

S. aureus pdf binding compounds may be designed “de novo” using either an empty binding site or optionally including some portion(s) of a known modifier(s). There are many de novo ligand design methods including, without limitation, LUDI (H.-J. Bohm, J. Comp. Aid. Molec. Design., 6:61-78 (1992); available from Molecular Simulations Inc., San Diego, Calif.); LEGEND (Y. Nishibata et al., Tetrahedron, 47:8985 (1991); available from Molecular Simulations Inc., San Diego, Calif.); LeapFrog (available from Tripos Associates, St. Louis, Mo.); and SPROUT (V. Gillet et al., J. Comput. Aided Mol. Design, 7:127-53 (1993); available from the University of Leeds, UK).

Once a compound has been designed or selected by the above methods, the efficiency with which that entity may bind to or interfere with an S. aureus pdf or S. aureus pdf-like active site may be tested and optimized by computational evaluation. For example, an effective S. aureus pdf or S. aureus pdf-like active site modifier must preferably demonstrate a relatively small difference in energy between its bound and free states (i.e., a small deformation energy of binding). Thus, the most efficient S. aureus pdf or S. aureus pdf-like active site modifiers should preferably be designed with a deformation energy of binding of not greater than about 10 kcal/mole; more preferably, not greater than 7 kcal/mole. S. aureus pdf or S. aureus pdf-like active site modifiers may interact with the active site in more than one conformation that is similar in overall binding energy. In those cases, the deformation energy of binding is taken to be the difference between the energy of the free entity and the average energy of the conformations observed when the modifier binds to the protein.

An entity designed or selected as binding to or interfering with an S. aureus pdf or S. aureus pdf-like active site may be further computationally optimized so that in its bound state it would preferably lack repulsive electrostatic interaction with the target enzyme and with the surrounding water molecules. Such non-complementary electrostatic interactions include repulsive charge-charge, dipole-dipole, and charge-dipole interactions.

Specific computer software is available in the art to evaluate compound deformation energy and electrostatic interactions. Examples of programs designed for such uses include: Gaussian 94, revision C (M. J. Frisch, Gaussian, Inc., Pittsburgh, Pa. (1995)); AMBER, version 4.1 (P. A. Kollman, University of California at San Francisco, (1995)); QUANTA/CHARMM (Molecular Simulations, Inc., San Diego, Calif. (1995)); Insight II/Discover (Molecular Simulations, Inc., San Diego, Calif. (1995)); DelPhi (Molecular Simulations, Inc., San Diego, Calif. (1995)); and AMSOL (Quantum Chemistry Program Exchange, Indiana University). These programs may be implemented, for instance, using a Silicon Graphics workstation such as an Indigo² with “IMPACT” graphics. Other hardware systems and software packages will be known to those skilled in the art.

Another approach encompassed by this invention is the computational screening of small molecule databases for chemical entities or compounds that can bind in whole, or in part, to a S. aureus pdf or S. aureus pdf-like active site. In this screening, the quality of fit of such entities to the binding site may be judged either by shape complementarity or by estimated interaction energy (E. C. Meng et al., J. Comp. Chem., 13:505-24 (1992)).

This invention also enables the development of chemical entities that can isomerize to short-lived reaction intermediates in the chemical reaction of a substrate or other compound that binds to or with S. aureus pdf. Time-dependent analysis of structural changes in S. aureus pdf during its interaction with other molecules is carried out. The reaction intermediates of S. aureus pdf can also be deduced from the reaction product in co-complex with S. aureus pdf. Such information is useful to design improved analogs of known modifiers of S. aureus pdf activity or to design novel classes of modifiers based on the reaction intermediates of the S. aureus pdf and modifier co-complex. This provides a novel route for designing S. aureus pdf modifiers with both high specificity and stability.

Yet another approach to rational drug design involves probing the S. aureus pdf crystal of the invention with molecules including a variety of different functional groups to determine optimal sites for interaction between candidate S. aureus pdf modifiers and the protein. For example, high resolution x-ray diffraction data collected from crystals soaked in or co-crystallized with other molecules allows the determination of where each type of solvent molecule sticks. Molecules that bind tightly to those sites can then be further modified and synthesized and tested for their hepes protease inhibitor activity (J. Travis, Science, 262:1374 (1993)).

In a related approach, iterative drug design is used to identify modifiers of S. aureus pdf activity. Iterative drug design is a method for optimizing associations between a protein and a compound by determining and evaluating the three-dimensional structures of successive sets of protein/compound complexes. In iterative drug design, crystals of a series of protein/compound complexes are obtained and then the three-dimensional structures of each complex is solved. Such an approach provides insight into the association between the proteins and compounds of each complex. This is accomplished by selecting compounds with inhibitory activity, obtaining crystals of this new protein/compound complex, solving the three dimensional structure of the complex, and comparing the associations between the new protein/compound complex and previously solved protein/compound complexes. By observing how changes in the compound affected the protein/compound associations, these associations may be optimized.

Pharmaceutical Compositions

Pharmaceutical compositions of this invention include a potential modifier of S. aureus pdf activity identified according to the invention, or a pharmaceutically acceptable salt thereof, and a pharmaceutically acceptable carrier, adjuvant, or vehicle. The term “pharmaceutically acceptable carrier” refers to a carrier(s) that is “acceptable” in the sense of being compatible with the other ingredients of a composition and not deleterious to the recipient thereof. Optionally, the pH of the formulation is adjusted with pharmaceutically acceptable acids, bases, or buffers to enhance the stability of the formulated compound or its delivery form.

Methods of making and using such pharmaceutical compositions are also included in the invention. The pharmaceutical compositions of the invention can be administered orally, parenterally, by inhalation spray, topically, rectally, nasally, buccally, vaginally, or via an implanted reservoir. Oral administration or administration by injection is preferred. The term parenteral as used herein includes subcutaneous, intracutaneous, intravenous, intramuscular, intra-articular, intrasynovial, intrasternal, intrathecal, intralesional, and intracranial injection or infusion techniques.

Dosage levels of between about 0.01 and about 100 mg/kg body weight per day, preferably between about 0.5 and about 75 mg/kg body weight per day of the S. aureus pdf inhibitory compounds described herein are useful for the prevention and treatment of S. aureus pdf mediated disease. Typically, the pharmaceutical compositions of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

In order that this invention be more fully understood, the following examples are set forth. These examples are for the purpose of illustration only and are not to be construed as limiting the scope of the invention in any way.

### EXAMPLES

### Example 1

### Analysis of the Structure of S. aureus pdf

Cloning and Expression

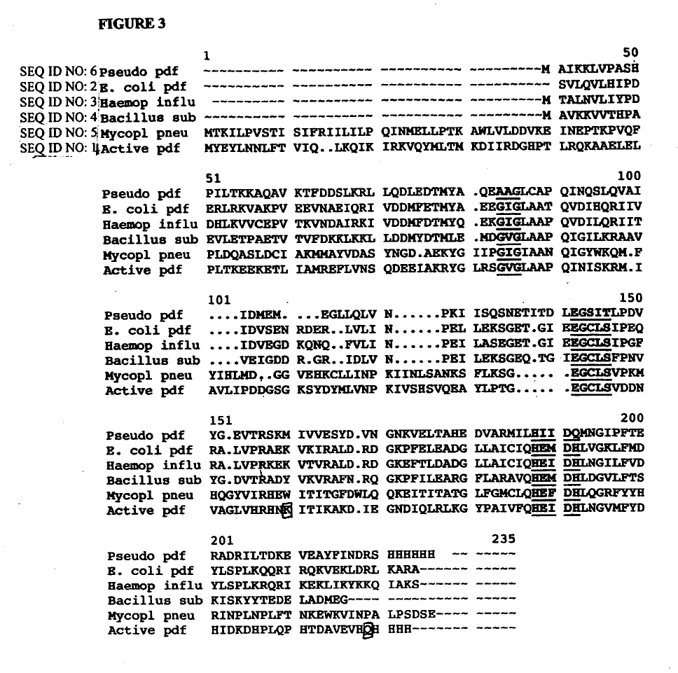

The plasmid containing the pdf insert was purified and used to transform a competent strain of E. coli JM 109. This cDNA clone used for protein expression and purification (R127K H186Q, highlighted in FIG. 3) contained two mutations. The second mutation is confirmed to be in the HIS6 tag (near the c-terminus) and has no effect on Km or Kcat. The gene encodes a total of 189 residues including a c-terminal hexahis tag.

The pdf protein was expressed using LB with ampicillin (100 mg/L) in both the seed and production media. LB was prepared using Bacto-tryptone (10 g), Bacto yeast (5 g), and NaCl (5 g) added per L of deioninzed water. The pH of the media was adjusted to 7.5 before sterilization with KOH. The LB broth was autoclaved for 20 minutes in 100 ml volumes in 500 ml wide mouth fermentation flasks. Ampicillin was filter sterilized and added just before innoculation. The 100 ml seed stock fermentations were carried out in 500 ml wide mouth flasks and were innoculated from agar cultures and were incubated overnight at 37° C. with agitation at 200 revolutions per minute (rpm). The seed fermentations were used to inoculate at 2% the 100 ml production fermentations which were also carried out in 500 ml wide mouth flasks. These fermentations were incubated with agitation at 200 rpm for slightly longer than 2 hours and were then induced (OD 660 nm reached 0.6). IPTG was added to a final concentration of 0.4 mM. The induced fermentations were continued for an additional 3.5 hours until the OD reached 3.0. Multiple fermentations produced a final harvest of 4-6 liters for purification.

For expression of selenomethionyl-Pdf, M9 glucose was utilized in 100 ml volumes containing ampicillin, thiamin, and PAS trace metal solution at 100 mg, 5 mg and 0.3 ml per liter of deionized water, respectively. Multiple shake flasks were used to attain the desired fermentation volume. Since JM109 is not a methionine auxotroph, incorporation of selenomethionine was accomplished through down regulation of methionine biosynthesis just prior to induction (Van Duyne, Standaert, 1993). The culture was grown in 500 ml wide mouth fermentation flasks at 37° C. with an agitation rate of 200 rpm until A600 reached ca. 0.5 unit. At this point, the following filter sterilized amino acids were added to achieve down-regulation. DL-selenomethione, L-lysine, L-threonine and L-phenylalanine were added to final concentrations of 120 micrograms/ml. L-leucine, L-isoleucine and L-valine were added to final concentrations of 60 micrograms/ml. After 15-20 minutes, protein expression was induced by the addition of filter sterilized IPTG added to a final concentration of 0.4 mM. Growth of the culture was continued as described for an additional 3 hours when A600 reached ca. 2 units. Cells were then harvested by centrifugation and stored at −80° C.

Purification

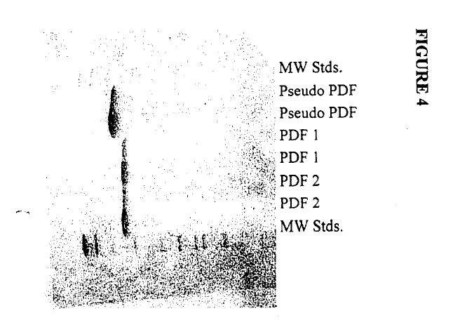

Cell paste from a two liter E. coli fermentation expressing S. aureus pdf was lysed in 50 mM Tris-HCl pH=8.0 with lysozyme dissolved at 1 mg/ml. The suspension sat on ice for 10 minutes and large strand DNA was broken by repeatedly shearing with a syringe and 19 gauge Needle. Cell extract was collected and centrifuged at 20500 rpm for 40-45 minutes at 5° C. Ni-NTA resin from Qiagen was equilibrated in lysis buffer (without lysozyme) and stirred into the cell extract. The suspension was poured into a column, washed expensively with lysis buffer and pdf was eluted with lysis buffer containing 200 mM imidazole. This protein designated as pdf1 was used for the first crystallization efforts, but required further purification (FIG. 4).

The eluate from the nickel column was concentrated by ultrafiltration with an Amicon stirred cell under nitrogen at room temperature. Two forms of pdf were resolved by anionic (Q fast flow, Pharmacia) exchange chromatography (without baseline resolution) as follows. A concentrated sample was injected onto a 1 mL column equilibrated with 25 mM Tris-HCl, pH=8.0. Proteins were resolved with a linear gradient of NaCl. The two forms of pdf were collected separately for further analysis. No differences in SDS-PAGE mobility or purity were observed. The first fraction had optimal activity while the second fraction had much less specific activity. Protein eluted in the early fraction of the gradient was collected and further concentrated in a stirred cell to the desired volume. Further purified pdf is referred to as pdf2, and improved purification is verified by Isoelectric focussing. Pdf was either delivered for crystallization experiments at this time, or was mixed with 50% glycerol and stored at −20° C. Enzyme assays have demonstrated that pdf is stable for over one year when stored in 50% glycerol at −20° C.

Selenomethionine pdf was purified for crystallization efforts as described above with the inclusion of 5 mM BME to reduce the chance of selenomethionine oxidation.

Protein Preparation

Protein was delivered immediately following concentration of peak material from the anion exchange column. The buffer contained 25 mM Tris pH=8.0 and approximately 50 mM NaCl. Protein was adjusted to 30 mg/ml and exchanged into buffer containing 25 mM Tris pH=8.0. Later batches of protein were received at a protein concentration of 60 mg/ml. This protein was diluted in half with water and frozen immediately in 50 microliter aliquots for later experiments.

Crystallization of S. aureus pdf

The first batch of pdf2 was received for crystallization. Crystallization experiments began with commercially available, random sparse matrix screens. Drops of 1 μL protein and 1 μL well solutions were set up in hanging drop vapor diffusion experiments at room temperature. Crystals grew in one week from 4 separate well conditions 6, 15, 18 and 22 of Hampton Crystal Screen I (Jancarik et al., J. Appl. Cryst., 24:409-11 (1991)). Follow-up grid screens were simultaneously set up to optimize each crystallization condition and are described below.

Hampton Research Crystal Screen I, #6

Hampton Screen I, condition 6 contains 30% PEG 4000, 0.2M MgCl₂ and 0.1M Tris pH=8.5. Original crystals grew as long thin needle clusters. Sitting drop vapor diffusion experiments were set up by mixing 2 microliters pdf+2 microliters reservoir solution. Crystals were optimized through a series of grid screens varying both PEG 4000 and MgCl₂ concentrations. Results from these screens produced larger rod crystals.

Micro-seeding was utilized in an attempt to grow individual crystals. Seed stocks were made by breaking off a large rod crystal and crushing it in 10 microliters of matching well solution. Serial dilutions of seed stocks were made to 10⁻⁴. Freshly mixed drops of protein and well solution containing 0.1M Tris pH=8.5, 0.075M MgCl₂ and varying amounts of PEG 4000 were seeded at setup with a cat whisker by successively streaking the whisker across one row. Single, chunky crystals grew within two weeks up to 0.35×0.35×0.7 micrometer. Large crystals often contained a channel down the middle of the crystal. Crystals were successfully stabilized and slowly transferred into a cryo-preservation solution containing 25% PEG 4000, 0.1M Tris pH=8.5, 0.1M MgCl₂ and 25% glycerol. Crystals were frozen in liquid nitrogen for cryogenic data collection.

Hampton Research Crystal Screen I, #15

Condition # 15 of Crystal Screen I was the second solution to produce crystals in the original screens. This solution contains 30% PEG 8000, 0.2M ammonium sulfate (A/S) and 0.1M Cacodylic acid pH=6.5. The original hit contained twinned crystalline rods that spread throughout the drop. The crystals were improved by varying both PEG 8000 and ammonium sulfate. Crystals improved significantly through micro-seeding. Crystals could be easily transferred to stabilization solution and slowly soaked into cryo-protective solution containing 22% PEG 8000, 0.2M ammonium sulfate, 0.1M cacodylic acid pH=6.5 and 25% glycerol for freezing.

Hampton Research Crystal Screen I, #18

A third solution to yield crystals was condition # 18 of Crystal Screen I. The solution is 20% PEG 8000, 0.1 m Na cacodylate pH=6.5 and 0.2M Mg acetate. Crystals grew as small rods that were very difficult to optimize. Seeding enabled the growth of a few large crystals, but crystals were very fragile. In many cases, crystals could not be stabilized without major crystal cracking. Despite these difficulties, a couple crystals were successfully soaked into cryo-solution containing 20% PEG 8000, 0.1 M NaCacodylate pH=6.5 and 0.2M MgAcetate and 25% glycerol and frozen for data collection.

Hampton Research Crystal Screen I, #22

Crystals also appeared in condition #22 which contains 30% PEG 4000, 0.1M Tris pH=8.5 and 0.2M sodium acetate. Crystals also grew as rod clusters and were optimized as described above for condition #6. Tweaking of the PEG 400 and Na acetate as well as micro-seeding produced large single rods grown from the bridge. Crystals were slowly soaked into cryo-solution of 0.3M Na acetate, 24% PEG 4000, 0.1M Tris pH=8.5 and 25% glycerol. Crystals diffracted well to 2.0 Å.

Selenomethionine pdf

Se-methionine pdf was prepared and initial crystallization experiments were set up in each of the four conditions as described above. An additional 5 mM BME was added to the reservoir solutions to reduce the chance of oxidation. Crystals from condition #6 were optimized through micro-seeding and produced sizable crystals. Crystals were prepared for low temperature data collection.

Data Collection, Space Group Determination

A crystal was grown from 28% PEG 8000, 0.1M cacodylic acid pH=6.5 and 0.1M ammonium sulfate and measured 0.1×0.1×0.5 micrometer. This crystal was the result of the follow up experiments from the Hampton I #15 hits. The crystal was frozen as described above for low temperature data collection. Data was collected on a single Hi Star at a detector distance of 18 cm and a temperature of 100° K. Frames of 300 seconds, 0.25° omega oscillation, and 2θ=15 were collected. Data was not processed because the crystal appeared obviously twinned.

Another crystal was grown from 16% PEG 8000, 0.1M Cacodylic Acid pH=6.5 and 0.4M Mg Acetate. This crystal was the result of the follow up experiments from the Hampton I #18 hits. The crystal was frozen and data was collected on the APS 17-ID beamline. The crystal diffracted to around 1.9 Å and about 400 frames of 0.5 degree oscillation data were collected (Table 5). The space group is C222₁ with unit cell parameters of a=94.296 Å, b=120.85 Å, c=47.88 Å, and α=β=γ=90°. Data collection ended since we were at the end of the run and the crystal was recovered at APS and refrozen for additional data collection. Data collection was continued on this crystal. Data was collected on a single Hi Star at detector distance of 12 cm and 300 seconds per frame. The 2θ angle was set to 15° with an omega oscillation of 0.25. Several water flow problems were encountered during data collection. This data was complete to around 2.7 Å (100% observed) with the I/sigma dropping below 2.0 for the higher resolution data. This data was not used for calculations. Molecular replacement was attempted using this data, but was unsuccessful.

A Se-methionine crystal was grown from 22.5% PEG 4000, 0.1M Tris pH=8.5 and 0.075M MgCl₂. This crystal was the result of the follow up experiments from the Hampton I #6 hits. Data was collected on a dual Hi Star at 12 cm and 100° K. Each frame oscillated 0.25° omega for 200 seconds at 2θ=−25°. Data collection statistics are summarized in Table 6. The space group is C222₁ with unit cell parameters of a=94.469 Å, b=121.965 Å, c=47.58 Å, and α=β=γ=90°. This crystal diffracted to around 2.0 Å resolution. This data set was used for molecular replacement studies, but these also failed to produce a good solution. This data suggested that a good data set could be obtained from these Se-Methionine crystals at APS.

Preliminary co-crystallization experiments began in an attempt to obtain a pdf complex with several leads as determined from screens. A crystal was grown in the presence of 10% DMSO and 2 mM of a potential inhibitor as well as the reservoir solution containing 20% PEG 4000, 0.1M Tris pH=8.5 and 0.1M MgCl₂. This crystal was the result of the follow up experiments from the Hampton I #6 hits. The crystal measured 0.28×0.28×0.98 micrometer and was frozen for low temperature data collection. Data was collected on a dual Hi Star at 100° K. The detector distance was 12 cm and 2θ=30°. Each frame of 0.25° omega oscillation was exposed for 200 seconds. The crystal diffracted to 1.9 Å and was of the C222¹ space group with unit cell parameters of a=94.95 Å, b=122.08 Å, c=47.73 Å, and α=β=γ=90°. Data statistics are summarized in Table 7. This data was used for refinement after the pdf structure was solved by MAD phasing, but a bound inhibitor was not observed.

Additional Se-methionine crystals were prepared for MAD data collection at APS. A crystal grew from 19% PEG 4000, 0.075M MgCl₂ and 0.1 m Tris pH=8.5.. This crystal was the result of the follow up experiments from the Hampton I #6 hits. A total of 3 data sets were collected on the 17-ID beamline at APS. The crystal to detector distance was 15 cm, 2θ=0 and each frame of 0.5° was exposed for 0.5 seconds. The ring current was 96.4 mA. A low data set was collected at a low λ=1.03321, an edge data set was collected at the adsorption edge of λ=0.0.97939, and a peak data set was collected at λ=0.97928. Data collection statistics are summarized in Table 8. The space group is C222₁, with unit cell parameters of a=94.113 Å, b=121.873 Å, c=47.579 Å, and α=β=γ=90°.

TABLE 5
Data collection statistics.

| Å | Obs | Theory % | Redund | Rsym | Pairs | % | Rshell | % | 2s |
|---|---|---|---|---|---|---|---|---|---|
| to 4.090 | 2042 | 2343 | 87.15 | 3.86 | 0.0651863 | 79.51 | 0.065 | 85.08 | 2.9 |
| to 3.247 | 4094 | 4584 | 89.31 | 3.99 | 0.0673737 | 81.52 | 0.069 | 62.43 | 4.1 |
| to 2.837 | 6182 | 6780 | 91.18 | 4.07 | 0.0675683 | 83.82 | 0.067 | 42.23 | 5.8 |
| to 2.578 | 8269 | 8975 | 92.13 | 4.11 | 0.0697634 | 85.06 | 0.079 | 26.72 | 7.8 |
| to 2.393 | 10326 | 11156 | 92.56 | 4.13 | 0.0719571 | 85.79 | 0.093 | 21.02 | 11.4 |
| to 2.252 | 12312 | 13339 | 92.30 | 4.08 | 0.07411360 | 85.16 | 0.114 | 17.26 | 12.4 |
| to 2.139 | 14308 | 15515 | 92.22 | 3.96 | 0.07713141 | 84.70 | 0.126 | 14.22 | 15.7 |
| to 2.046 | 16170 | 17685 | 91.43 | 3.82 | 0.07914655 | 82.87 | 0.146 | 12.01 | 18.3 |
| to 1.967 | 17759 | 19839 | 89.52 | 3.70 | 0.08115709 | 79.18 | 0.195 | 8.90 | 22.9 |
| to 1.899 | 19024 | 22028 | 86.36 | 3.60 | 0.08316453 | 74.69 | 0.242 | 6.89 | 28.1 |

TABLE 6
Data collection statistics for data with I/sigma greater than 2.

| Resolution | Ref | Ref | | | |
|---|---|---|---|---|---|
| Å | Possible | Observed | observations | R-factor | I/sigma |
| 3.76 | 3005 | 2791 | 21889 | 8.88 | 58.19 |
| 2.98 | 2871 | 2629 | 23125 | 9.73 | 43.57 |
| 2.61 | 2852 | 2453 | 18757 | 14.62 | 22.66 |
| 2.37 | 2843 | 2340 | 11384 | 11.83 | 13.50 |
| 2.20 | 2811 | 2148 | 9136 | 23.25 | 9.48 |
| 2.07 | 2809 | 1427 | 4412 | 15.93 | 6.45 |
| | 17191 | 13788 | 88703 | 10.29 | 28.55 |

TABLE 7
Data collection statistics for data with I/sigma greater than 2.

| Resolution | Ref | Ref | | | |
|---|---|---|---|---|---|
| Å | Possible | Observed | observations | R-factor | I/sigma |
| 3.61 | 3392 | 3160 | 21173 | 2.27 | 70.5 |
| 2.87 | 3259 | 3117 | 15625 | 3.99 | 37.7 |
| 2.51 | 3214 | 2881 | 8737 | 6.74 | 16.4 |
| 2.28 | 3201 | 2686 | 6898 | 9.37 | 10.8 |
| 2.11 | 3209 | 2520 | 5961 | 10.51 | 8.87 |
| 2.00 | 3176 | 1945 | 4074 | 11.24 | 6.90 |
| | 19451 | 16309 | 62468 | 3.70 | 27.7 |

TABLE 8
Data collection statistics.

| Coverage Statistics | | | | | . . . Shell | | | | | |
|---|---|---|---|---|---|---|---|---|---|---|
| Angstrms | #Obs | Theory % | Compl | Redund | Rsym | Pairs % | Pairs | Rshell | #Sigma % | <2s |

stats low

| | | | | | | | | | | |
|---|---|---|---|---|---|---|---|---|---|---|
| to 4.091 | 2265 | 2343 | 96.67 | 4.46 | 0.028 | 2072 | 88.43 | 0.028 | 84.43 | 2.0 |
| to 3.247 | 4503 | 4588 | 98.15 | 4.82 | 0.030 | 4249 | 92.61 | 0.032 | 64.10 | 2.4 |
| to 2.837 | 6720 | 6792 | 98.94 | 5.10 | 0.033 | 6444 | 94.88 | 0.041 | 38.38 | 4.0 |
| to 2.578 | 8925 | 8992 | 99.25 | 5.27 | 0.035 | 8646 | 96.15 | 0.048 | 26.64 | 5.6 |
| to 2.393 | 11131 | 11185 | 99.52 | 5.35 | 0.037 | 10850 | 97.00 | 0.053 | 20.55 | 8.2 |
| to 2.252 | 13312 | 13363 | 99.62 | 5.23 | 0.038 | 12969 | 97.05 | 0.056 | 17.44 | 10.4 |
| to 2.139 | 15489 | 15533 | 99.72 | 5.07 | 0.039 | 14978 | 96.43 | 0.062 | 14.57 | 13.6 |
| to 2.046 | 17604 | 17725 | 99.32 | 4.92 | 0.040 | 16869 | 95.17 | 0.067 | 12.58 | 15.0 |
| to 1.967 | 19626 | 19868 | 98.78 | 4.74 | 0.041 | 18446 | 92.84 | 0.081 | 9.67 | 18.8 |
| to 1.899 | 21519 | 22066 | 97.52 | 4.54 | 0.042 | 19661 | 89.10 | 0.107 | 7.97 | 22.2 |

stats edge

| | | | | | | | | | | |
|---|---|---|---|---|---|---|---|---|---|---|
| to 4.091 | 2249 | 2343 | 95.99 | 3.74 | 0.034 | 1577 | 67.31 | 0.034 | 81.38 | 2.1 |
| to 3.247 | 4478 | 4588 | 97.60 | 4.17 | 0.037 | 3455 | 75.31 | 0.039 | 60.12 | 2.6 |
| to 2.837 | 6688 | 6792 | 98.47 | 4.51 | 0.043 | 5468 | 80.51 | 0.061 | 33.75 | 5.2 |
| to 2.578 | 8892 | 8992 | 98.89 | 4.76 | 0.048 | 7550 | 83.96 | 0.081 | 22.55 | 7.3 |
| to 2.393 | 11088 | 11185 | 99.13 | 4.94 | 0.053 | 9682 | 86.56 | 0.095 | 16.88 | 10.6 |
| to 2.252 | 13278 | 13363 | 99.36 | 5.03 | 0.057 | 11842 | 88.62 | 0.101 | 14.05 | 12.9 |
| to 2.139 | 15478 | 15533 | 99.65 | 4.97 | 0.060 | 13963 | 89.89 | 0.107 | 11.68 | 17.4 |
| to 2.046 | 17649 | 17725 | 99.57 | 4.86 | 0.062 | 15967 | 90.08 | 0.113 | 10.17 | 18.8 |
| to 1.967 | 19761 | 19868 | 99.46 | 4.76 | 0.064 | 17876 | 89.97 | 0.129 | 7.54 | 24.6 |
| to 1.899 | 21904 | 22066 | 99.27 | 4.62 | 0.065 | 19621 | 88.92 | 0.152 | 5.87 | 29.9 |

stats peak

| | | | | | | | | | | |
|---|---|---|---|---|---|---|---|---|---|---|
| to 4.091 | 2280 | 2343 | 97.31 | 3.60 | 0.038 | 1594 | 68.03 | 0.038 | 81.02 | 2.3 |
| to 3.247 | 4480 | 4588 | 97.65 | 4.04 | 0.040 | 3446 | 75.11 | 0.041 | 59.39 | 3.0 |
| to 2.837 | 6677 | 6792 | 98.31 | 4.41 | 0.046 | 5373 | 79.11 | 0.063 | 33.15 | 5.9 |
| to 2.578 | 8881 | 8992 | 98.77 | 4.67 | 0.051 | 7393 | 82.22 | 0.081 | 22.29 | 7.5 |
| to 2.393 | 11072 | 11185 | 98.99 | 4.87 | 0.056 | 9452 | 84.51 | 0.095 | 16.80 | 10.8 |
| to 2.252 | 13247 | 13363 | 99.13 | 4.97 | 0.060 | 11527 | 86.26 | 0.105 | 13.87 | 13.5 |
| to 2.139 | 15449 | 15533 | 99.46 | 4.92 | 0.063 | 13605 | 87.59 | 0.116 | 11.72 | 17.7 |
| to 2.046 | 17637 | 17725 | 99.50 | 4.81 | 0.066 | 15657 | 88.33 | 0.123 | 10.11 | 19.0 |
| to 1.967 | 19798 | 19868 | 99.65 | 4.70 | 0.069 | 17642 | 88.80 | 0.143 | 7.63 | 24.5 |
| to 1.899 | 22008 | 22066 | 99.74 | 4.56 | 0.071 | 19528 | 88.50 | 0.168 | 6.04 | 29.2 |

Phase Determination and Refinement

The structure of S. aureus pdf was determined by multiple anomalous dispersion (MAD) using synchrotron radiation. The MAD data set included data to 1.9 Å resolution. Anomalous difference Patterson maps revealed the expected six selenium sites for a single protein molecule in the asymmetric unit. An excellent well-phased map to 1.9 Å resolution was produced into which the protein model could be easily built. However, XPLOR refinement of this model did not result in a model with an R-factor below 30%. This was difficult to understand since the overall map quality was excellent and there was little remaining difference density unaccounted for. This refinement effort was eventually discontinued in favor of a second data set. The 2.0 Å resolution data from the pdf crystal was used for the refinement of the structure. These data did refine well and a final R-factor of 18.6% for this model with good geometry was obtained (Table 9).

The X-ray data for the MAD phasing of pdf was collected at the Advanced Photon Source and consisted of three separate wavelength experiments centered about the Selenium edge (low, 1.03321 Å; edge, 0.97939 Å; high, 0.97928 Å). Each of the data sets were indexed and integrated separately. The data sets were scaled together using the program SCALEIT in the CCP4 Program Suite (Collaborative Computational Project N4, Acta Cryst., D50:760-63 (1994)). Patterson maps revealed six selenium sites whose locations were determined and refined by direct methods using SHELX (Sheldrick et al., Acta Cryst., B51:423-31 (1995)). Heavy atom refinement and phase calculations were carried out using MLPHARE from CCP4 with all the data from 10 to 1.9 Å resolution. The resulting electron density map was readily interpreted and a model built. A density modified map was also calculated (MLPHARE), but the maps were not very different. Model building was done with the program CHAIN (Sack, J. Mol. Graphics, 6:224-25 (1988)) and LORE (Finzel, Meth. Enzymol., 277:230-42 (1997)). Initial refinement was carried out with XPLOR (Brunger AT. X-PLOR version 3.1: A system for X-ray crystallography and NMR. New Haven: Yale Univ. Press, (1992)). However, the R-factor failed to fall below 30% after several cycles and with the inclusion of many waters. At that point the refinement of this data set was discontinued in favor of another data set.

TABLE 9
Data collection and phasing statistics

| | λ 1.03321 Å | λ 0.97939 Å | λ 0.97928 Å |
|---|---|---|---|
| Resolution | 1.9 Å | 1.9 Å | 1.9 Å |
| Average redundancy | 4.5 | 4.5 | 4.5 |
| # unique reflections | 21519 | 21904 | 22008 |
| % completeness | 97.5% | 99.3% | 99.7% |
| R_{sym}^{♯} | 0.042 | 0.065 | 0.071 |
| R_{sym} (1.96-1.89 shell) | 0.107 | 0.152 | 0.168 |
| R_{cullis} acentrics | 1.70 (19034 refs) | 0.87 (18878 refs) | 0.57 (18899 refs) |
| R_{cullis} anomalous | 0.98 (19178 refs) | 0.64 (18418 refs) | 0.58 (18679 refs) |
| Phasing Power | | | |
| Centrics | — | 0.70 | 1.83 |
| Acentrics | — | 0.80 | 2.15 |
| Mean FOM | overall | centric | acentric |
| Before solvent flattening | 0.714 (21048ref) | 0.627 (2014 refs) | 0.724 (19034 refs) |
| After solvent flattening | 0.788 | — | — |

Refinement of the Data Set.

This data was used for the further refinement of the native pdf structure. The partially refined model derived from the MAD map was rotated to an arbitrary initial position, stripped of water and cations, and used for molecular replacement (XPLOR). The rotation solutions were filtered with PC-refinement (Brunger, Acta Crystallogr., A46:46-47 (1990)). The highest rotation function peak also resulted in the highest PC-filtered peak (PC=0.194). The position of the rotated monomer was obtained by a translation search (again the highest peak in the map and 15.6 sigma above the mean). The solution obtained was consistent with the position of the molecule in the MAD map and had an initial R-factor of 39.6% for data from 20-2.5 Å resolution (9235 reflections). This structure was further refined with XPLOR positional refinement and waters and a Zinc atom incorporated into the model. The R-factor dropped to 21% with a Free-R-factor of just over 25%. A final cycle of refinement and rebuilding was employed using PROLSQ (Hendrickson et al., “Stereochemically restrained crystallographic least-squares refinement of macromolecule structures” in Biomolecular Structure, Function and Evolution, (R. Srinivasan, ed. 43-57) Pergamon Press, Oxford UK (1980)) which resulted in a final R-factor of 18.62% for 16266 reflections, 10-2.0 Å resolution data. The final agreement statistics (Table 10) and Ramachandran plot revealed a well-refined structure and are included below. Additional statistics were generated with PROCHECK (Laskowski et al., J. Appl. Cryst., 26:283-91 (1993)). A comparison of the initial MAD map and the final refined map was produced in CHAIN.

TABLE 10
Final model agreement statistics for PDF data set.

| | |
|---|---|
| Resolution: | 2.00 Angstrom |
| R-value: | 18.62% for 16,266 reflections (2sigma) |
| Atoms | 1725 (305 waters); 1 Zinc |
| Mean B-factor | 15.0 Å² |

Final Model rmsd from expected for restraint class:

| | | |
|---|---|---|
| Distances: | 1-2 bonds | 0.018 (0.030) |
| | 1-3 bond angle | 0.031 (0.040) |
| | 1-4 torsional | 0.029 (0.050) |
| Planes | peptides | 0.016 (0.030) |
| | Other | 0.014 (0.030) |
| | chiral volumes | 0.204 (0.250) |
| NonBonded | 1-4 | 0.174 (0.300) |
| | H-bond | 0.204 (0.300) |
| | other | 0.172 (0.300) |
| Thermal | 1-2 mainchain | 1.033 (1.500) |
| | 1-3 | 1.676 (3.000) |
| | 1-2 sidechain | 2.109 (2.000) |
| | 1-3 sidechain | 3.293 (4.000) |

Comparison of S. aureus and E. coli pdf Structures.

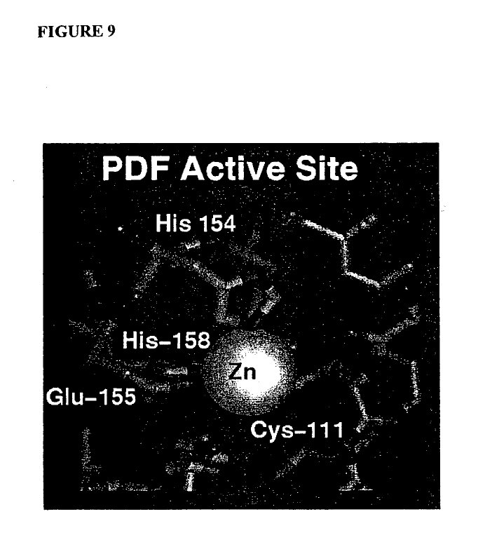

The final S. aureus pdf and the E. coli pdf complex with (S)-2-O-(H-phosphonoxy)-L-caproyl-L-leucyl-p-nitroanilide (PCLNA) (Hao et al., Biochemistry, 38: 4712-19 (1999)) were compared using SUPERPDB (Finzel, unpublished). FIGS. 6 and 11 were produced with MOLSCRIPT (Kraulis, J. Appl. Cryst., 24:946-50 (1991)) and Raster 3D (Merritt et al., Acta Cryst., D50:869-73 (1994)). FIGS. 8, 9, 12 and 13 were prepared with MOSAIC2. FIGS. 7 and 14 were prepared with CHAIN using PLOT.

### Example 2

### PDF Complexes with Actinonin and Thiorphan

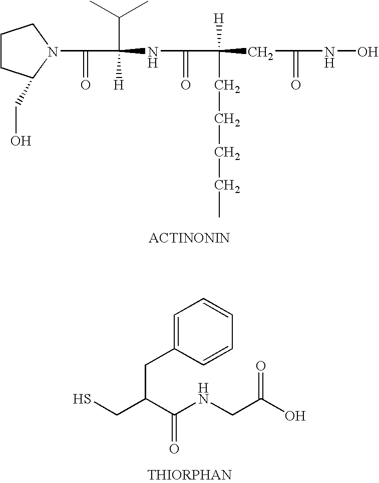

Methods

The peptide deformylase enzyme from S. aureus was cloned, expressed and purified as previously described. Protein was provided for crystallization in 25 mM Tris-HCl pH=8.0 and 50 mM NaCl at a protein concentration between 30 to 60 mg ml⁻¹. Protein samples were diluted to 30 mg ml⁻¹ with matching buffer solution, snap frozen in liquid nitrogen and stored at −80° C. for later crystallization experiments. Two previously reported PDF inhibitors were chosen for co-crystallization studies; actinonin with an IC₅₀=3 nM and thiorphan with an IC₅₀=12 μM. PDF protein was mixed with 5 mM thiorphan or 2 mM actinonin and incubated on ice for 1 hour.

All inhibitor stock solutions were prepared at 100 mM in 100% dimethyl sulfoxide. Sitting drop vapour diffusion experiments were set-up by mixing 4 μl protein+4 μl well solution containing 17-28% PEG 4000, 0.1M MgCl₂, and 0.05 M Tris pH=8.5. After 1-2 weeks, heavy precipitation and needle clusters were observed at the higher PEG 4000 concentrations, but single crystals did not grow spontaneously. Single, rod-shaped crystals were achieved with both ligands as a result of micro-seeding with a 10⁻⁴ diluted ligand-free seed stock 1 day after set-up. Single crystals grew within two weeks. Crystals were cryopreserved as described for the ligand-free crystals in 25% PEG 4000, 0.1M MgCl₂, 0.05 M Tris pH=8.5, 25% glycerol and 2-5 mM ligand.

Data Collection and Processing

PDF/inhibitor complex data sets were collected with synchrotron X-rays tuned to 1.200 Å. The data set were collected using a MAR CCD detector with MARccd data acquisition software (MAR, USA; M. Blum, unpublished). Two-dimensional images were integrated and scaled with HKL2000 (Otwinowski & Minor, 1997). Data collection parameters and processing statistics for the data sets and are shown in Table 1. The thiorphan and actinonin co-crystals grow with similar unit cell parameters and in the same space group as the native crystals, C222₁. The co-crystals diffract strongly to 1.8 Å and 2.0 Å. There is one molecule per asymmetric unit in the C222₁, crystals and the specific volume V_{M} is 3.585 Å³Da⁻¹ with a solvent content of 65.7%.

TABLE 11
Data collection statistics for PDF complexes with Thiorphan and
Actinonin.

| | Thiorphan | Actinonin | |
|---|---|---|---|
| wavelength (Å) | 1.2000 | 1.2000 | 1.2000 |
| crystal to detector distance (cm) | 10.0 | 1.2 | 12.0 |
| exposure time (seconds) | 3.0 | 5.0 | 2.0 |
| number of frames | 120 | 120 | 116 |
| frame width(°) | 1.0 | 1.00 | 1.00 |
| 2theta (°) | 0.0 | 0.0 | 60 mm |

| Space group | C222₁ | C222₁ |
|---|---|---|
| Unit-cell parameters (Å, °) | a = 94.1 | a = 93.6 |
| | b = 121.9 | b = 121.8 |
| | c = 47.6 | c = 48.2 |
| Resolution range (Å) | 20.0-1.8 | 20.0-1.6 |
| Number of observations | 121348 | 150703 |
| Number of unique reflections | 26700 | 37012 |
| Redundancy | 4.5 | 4.0 |
| Completeness (%) | 98.7(91.3) | 91.8(78.6) |
| R_{sym} (%) | 5.1(21.3) | 4.0(7.5) |
| mosaicity (°) | 0.573 | 0.473 |

Structure Determination of Actinonin Complex

The structure of the actinonin complex was solved by molecular replacement using the native Se-Met structure. CNX was used to carry out rigid body refinement, followed by cycles of SA-refinement and water addition. Water was excluded from the region of the inhibitor until the R-factor dropped below 25%. After some additional manual adjustment of the protein structure, difference Fourier maps were calculated. These maps were visualized with CHAIN and were consistent with the presence of actinonin in the PDF active site. The final structure used data to 1.6 Å resolution and had an R-free 20.6% of and an R-factor of 19.3%. The final agreement statistics reveal a well-refined structure. The final model consisted of 1454 protein atoms, which encompassed protein residues Met-1 thru H184 from the purification tag. The remaining residues from the purification tag were not ordered. In addition the final model consists of the 390 water molecules, 27 inhibitor atoms, and 1 zinc atom. The Ramachandran plot for the structure shows that all residues are with the allowed regions of phi/psi space with 93% within the most favoured regions.

Structure Determination of Thiorphan Complex

The structure of the thiorphan complex was solved and refined. Molecular replacement was carried out using the high-resolution refined structure of the actinonin complex (aps213) without waters or inhibitor as the starting model. A final R-free of 22.8% and an R-factor of 19.0% were obtained for data from 17 to 1.8 Å resolution. The final agreement statistics reveal a well-refined structure. The final model consisted of 1454 protein atoms, which encompassed protein residues Met-1 thru H184 from the purification tag. The remaining residues from the purification tag were not ordered. In addition the final model consists of the 393 water molecules, 17 inhibitor atoms, and 1 zinc atom. The Ramachandran plot for the structure shows that all residues are with the allowed regions of phi/psi space with 93% within the most favoured regions.

Results and Discussion

Actinonin Structure

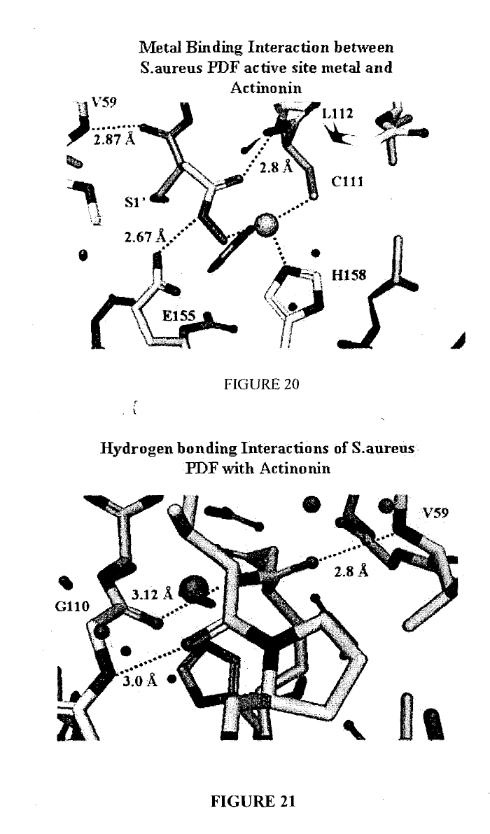

Actinonin is a peptidic inhibitor that occupies S1′, S2′ and the S3′ subsites. See for instance, Schechter et. al. BBRC 78, 157 (1967). The inhibitor places a hydrophobic alkyl chain in the S1′ subsite that would normally expect a methionine residue in the typical substrate. The Valine residue in S2′ is solvent exposed. The pyrrolidinyl group in S3′ is mostly solvent exposed with the 2-hydroxymethyl making a water mediated hydrogen bond to sidechain of theonine-107. The inhibitor binds to the metal and protein with the hydroxymate group and makes additional hydrogen bonds with L112, V59 and G110. A water molecule that was the fourth ligand to the metal is displace by hydroxymate binding. Non-bonded contacts between actinonin and PDF are numerous. These are summarized in Table 12. An electrostatic analysis of the active site surface indicates appropriately charged surface features that correspond to the points of inhibitor hydrogen bonding. The S1′ subsite is mostly of neutral charge as expected, but appears to have some negative character. This may arise from the conserved salt bridge between R124 and E109 that forms one side of the binding pocket. See FIGS. 20 and 21.

Thiorphan (PNU-276729) Structure

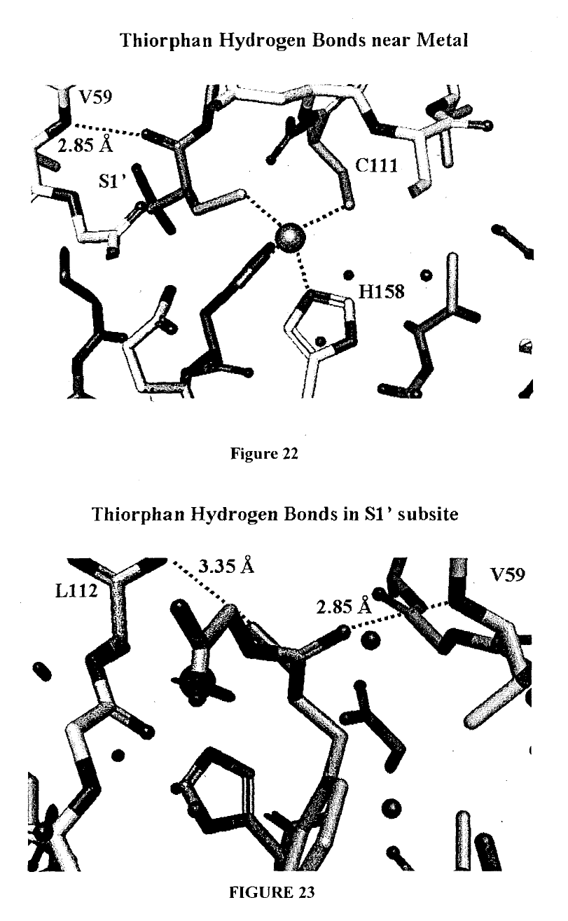

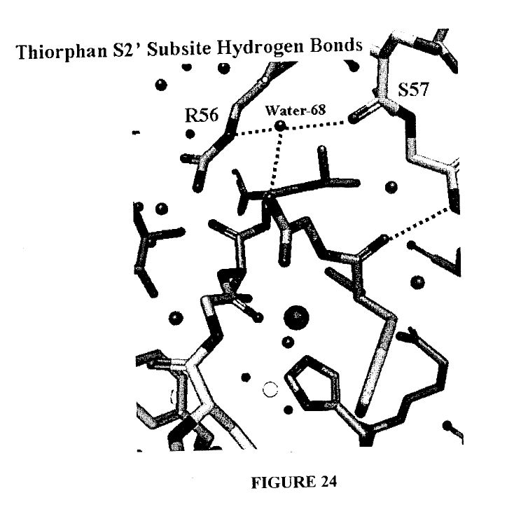

Thiorphan is a thiol-based peptidic inhibitor that occupies S1′ and S2′ subsites. The thiol group is the fourth ligand for the active site metal. In addition the sulfur atom is 3.3 Å away from the backbone amide of L112 and may make a weak interaction. The inhibitor carbonyl group make a good hydrogen bond with the backbone amide of V59. The inhibitor projects a benzyl group into S1′ and represents the first report of an aromatic group in this subsite. The acid group in S2′ makes indirect hydrogen bonded to the protein through water-68. Longer hydrogen bonds/weak electrostatic interactions with R56 are noted as well. Non-bonded contacts are summarized in Table 4. Electrostatic interactions of thiorphan with PDF indicate some complementarity's with polar groups. The inhibitor amide does not make a good interaction. The G110 carbonyl is 3.35 Å distant from the amide and the C-O-N angle is a poor 98°. See FIGS. 22-24.

TABLE 12
Comparison of the non-bonded contacts between Actinonin or Thiorphan
and PDF residues by 4, 7 and 10 Å shells.

| | Actinonin | | | Thiorphan | | |
|---|---|---|---|---|---|---|
| Residue | Å | 7 Å | 10 Å | 4 Å | 7 Å | 10 Å |
| MET 4 A | | | * | | | * |
| ILE 7 A | | | * | | | * |
| ILE 8 A | | | * | | | * |
| ARG 9 A | | | * | | | * |
| LEU 15 A | | | * | | | * |
| MET 37 A | | | * | | | |
| PHE 40 A | | | * | | | * |
| LEU 41 A | | * | * | | * | * |
| SER 44 A | | | * | | | * |
| GLN 45 A | | | * | | | * |
| LEU 55 A | | | * | | | * |
| ARG 56 A | * | * | * | * | * | * |
| SER 57 A | * | * | * | * | * | * |
| GLY 58 A | * | * | * | * | * | * |
| VAL 59 A | * | * | * | * | * | * |
| GLY 60 A | * | * | * | * | * | * |
| LEU 61 A | | * | * | | * | * |
| ALA 62 A | | * | * | | * | * |
| ALA 63 A | | | * | | | * |
| PRO 64 A | | | * | | | * |
| GLN 65 A | * | * | * | * | * | * |
| ILE 66 A | | | * | | | * |
| MET 72 A | | | * | | | |
| ILE 73 A | | | * | | | * |
| ALA 74 A | | * | * | | | * |
| VAL 75 A | | | * | | | * |
| LEU 76 A | | | * | | | * |
| ILE 77 A | | | * | | | * |
| PRO 78 A | | | * | | | * |
| ASP 80 A | | | * | | | |
| ALA 103 A | | | * | | | * |
| TYR 104 A | | | * | | | * |
| LEU 105 A | * | * | * | * | * | * |
| PRO 106 A | | | * | | | * |
| THR 107 A | | * | * | | | * |
| GLY 108 A | | * | * | | * | * |
| GLU 109 A | * | * | * | * | * | * |
| GLY 110 A | * | * | * | * | * | * |
| CYS 111 A | * | * | * | * | * | * |
| LEU 112 A | * | * | * | * | * | * |
| SER 113 A | | * | * | | * | * |
| VAL 114 A | | * | * | | | * |
| ASP 115 A | | | * | | | * |
| ASP 116 A | | | * | | | * |
| ASN 117 A | | * | * | | * | * |
| VAL 118 A | | | * | | | * |
| GLY 120 A | | | * | | | * |
| VAL 122 A | | | * | | | * |
| ARG 124 A | | * | * | | * | * |
| GLY 146 A | | | * | | | * |
| TYR 147 A | * | * | * | * | * | * |
| PRO 148 A | | | * | | | * |
| ALA 149 A | | | * | | | * |
| ILE 150 A | | * | * | * | * | * |
| VAL 151 A | | * | * | * | * | * |
| PHE 152 A | | * | * | | * | * |
| GLN 153 A | | | * | | * | * |
| HIS 154 A | * | * | * | * | * | * |
| GLU 155 A | * | * | * | * | * | * |
| ILE 156 A | | | * | | | * |
| ASP 157 A | | | * | | | * |
| HIS 158 A | * | * | * | | * | * |
| LEU 159 A | | | * | | | |
| VAL 162 A | | | * | | | * |
| MET 163 A | | | * | | | * |
| PHE 164 A | | * | * | | * | * |
| TYR 165 A | | | | | | * |
| HIS 167 A | | | * | | | * |
| VAL 183 A | | | * | | | ★ |
| HIS 184 A | | | * | | | |
| Zinc | * | * | * | * | * | * |

The complete disclosure of all patents, patent applications including provisional applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described; many variations will be apparent to one skilled in the art and are intended to be included within the invention defined by the claims.

### Sequence Listing Free Text

SEQ ID NO:1 Staphylococcus aureus peptide deformylase with C-terminal 6×His tag

SEQ ID NO:2 Escherichia coli peptide deformylase

SEQ ID NO:3 Haemophilis influenzae peptide deformylase

SEQ ID NO:4 Bacillus subtilis peptide deformylase

SEQ ID NO:5 Mycoplasma pneumoniae peptide deformylase

SEQ ID NO:6 Staphylococcus aureus def1 gene (Pseudo pdf)

TABLE 1
Structure Coordinates for S. aureus pdf

| | | | | | | |
|---|---|---|---|---|---|---|
| CRYST1 | 94.950 | 122.080 | 47.730 | 90.00 | 90.00 | 90.00 |

| | | | | |
|---|---|---|---|---|
| SCALE1 | 0.010532 | 0.000000 | 0.000000 | 0.000000 |
| SCALE2 | 0.000000 | 0.008191 | 0.000000 | 0.000000 |
| SCALE3 | 0.000000 | 0.000000 | 0.020951 | 0.000000 |

| | | | | | | | | | |
|---|---|---|---|---|---|---|---|---|---|
| ATOM | 1 | N | MET | 1 | 34.916 | 34.289 | 28.962 | 1.00 | 19.94 |
| ATOM | 2 | CA | MET | 1 | 34.532 | 33.707 | 30.269 | 1.00 | 17.68 |
| ATOM | 3 | CB | MET | 1 | 34.906 | 34.864 | 31.043 | 1.00 | 22.02 |
| ATOM | 4 | CG | MET | 1 | 34.249 | 35.660 | 31.315 | 1.00 | 26.50 |
| ATOM | 5 | SD | MET | 1 | 34.946 | 36.841 | 32.629 | 1.00 | 21.74 |
| ATOM | 6 | CE | MET | 1 | 34.539 | 36.320 | 34.127 | 1.00 | 24.64 |
| ATOM | 7 | C | MET | 1 | 33.437 | 33.258 | 30.418 | 1.00 | 14.02 |
| ATOM | 8 | O | MET | 1 | 32.276 | 33.920 | 29.938 | 1.00 | 26.24 |
| ATOM | 9 | N | LEU | 2 | 32.981 | 31.938 | 30.879 | 1.00 | 7.75 |
| ATOM | 10 | CA | LEU | 2 | 31.816 | 31.467 | 31.565 | 1.00 | 8.41 |
| ATOM | 11 | CB | LEU | 2 | 32.031 | 30.005 | 32.023 | 1.00 | 8.48 |
| ATOM | 12 | CO | LEU | 2 | 32.268 | 28.961 | 30.866 | 1.00 | 7.73 |
| ATOM | 13 | CD1 | LEU | 2 | 32.614 | 27.626 | 31.509 | 1.00 | 9.08 |
| ATOM | 14 | CD2 | LED | 2 | 30.932 | 28.747 | 30.133 | 1.00 | 8.57 |
| ATOM | 15 | C | LEU | 2 | 31.402 | 32.379 | 32.708 | 1.00 | 9.38 |
| ATOM | 16 | O | LED | 2 | 32.278 | 32.943 | 33.415 | 1.00 | 10.57 |
| ATOM | 17 | N | THR | 3 | 30.099 | 32.656 | 32.839 | 1.00 | 9.70 |
| ATOM | 18 | CA | THR | 3 | 29.624 | 33.466 | 33.943 | 1.00 | 10.89 |
| ATOM | 19 | CB | THR | 3 | 29.238 | 34.900 | 33.661 | 1.00 | 10.34 |
| ATOM | 20 | OG1 | THR | 3 | 28.067 | 34.943 | 32.811 | 1.00 | 13.27 |
| ATOM | 21 | CG2 | THR | 3 | 30.363 | 35.684 | 33.051 | 1.00 | 9.36 |
| ATOM | 22 | C | THR | 3 | 28.428 | 32.714 | 34.511 | 1.00 | 12.61 |
| ATOM | 23 | O | THR | 3 | 28.150 | 31.586 | 34.034 | 1.00 | 12.67 |
| ATOM | 24 | N | MET | 4 | 27.740 | 33.297 | 35.478 | 1.00 | 13.52 |
| ATOM | 25 | CA | MET | 4 | 26.570 | 32.603 | 36.027 | 1.00 | 12.96 |
| ATOM | 26 | CB | MET | 4 | 26.007 | 33.362 | 37.225 | 1.00 | 12.16 |
| ATOM | 27 | CG | MET | 4 | 26.954 | 33.492 | 38.401 | 1.00 | 12.58 |
| ATOM | 28 | SD | MET | 4 | 27.512 | 31.883 | 38.995 | 1.00 | 12.58 |
| ATOM | 29 | CE | MET | 4 | 25.972 | 31.153 | 39.545 | 1.00 | 11.19 |
| ATOM | 30 | C | MET | 4 | 25.497 | 32.343 | 34.975 | 1.00 | 13.01 |
| ATOM | 31 | O | MET | 4 | 24.627 | 31.474 | 35.142 | 1.00 | 11.54 |
| ATOM | 32 | N | LYS | 5 | 25.475 | 33.147 | 33.917 | 1.00 | 13.68 |
| ATOM | 33 | CA | LYS | 5 | 24.490 | 32.976 | 32.902 | 1.00 | 14.22 |
| ATOM | 34 | CS | LYS | 5 | 24.163 | 34.124 | 31.992 | 1.00 | 16.93 |
| ATOM | 35 | CG | LYS | 5 | 25.243 | 34.788 | 31.252 | 1.00 | 18.60 |
| ATOM | 36 | CD | LYS | 5 | 24.807 | 35.647 | 30.112 | 1.00 | 19.21 |
| ATOM | 37 | CE | LYS | 5 | 23.755 | 36.633 | 30.201 | 1.00 | 17.56 |
| ATOM | 38 | NZ | LYS | 5 | 23.653 | 37.497 | 28.989 | 1.00 | 17.28 |
| ATOM | 39 | C | LYS | 5 | 24.684 | 31.690 | 32.119 | 1.00 | 13.07 |
| ATOM | 40 | O | LYS | 5 | 23.720 | 31.241 | 31.521 | 1.00 | 12.87 |
| ATOM | 41 | N | ASP | 6 | 25.880 | 31.127 | 32.131 | 1.00 | 12.68 |
| ATOM | 42 | CA | ASP | 6 | 26.110 | 29.855 | 31.443 | 1.00 | 10.98 |
| ATOM | 43 | CB | ASP | 6 | 27.608 | 29.644 | 31.134 | 1.00 | 11.64 |
| ATOM | 44 | CG | ASP | 6 | 28.053 | 30.725 | 30.147 | 1.00 | 12.48 |
| ATOM | 45 | OD1 | ASP | 6 | 27.837 | 30.487 | 28.933 | 1.00 | 13.27 |
| ATOM | 46 | OD2 | ASP | 6 | 28.505 | 31.815 | 30.591 | 1.00 | 11.62 |
| ATOM | 47 | C | ASP | 6 | 25.640 | 28.730 | 32.353 | 1.00 | 11.50 |
| ATOM | 48 | O | ASP | 6 | 25.445 | 27.605 | 31.881 | 1.00 | 11.74 |
| ATOM | 49 | N | ILE | 7 | 25.501 | 29.000 | 33.628 | 1.00 | 11.13 |
| ATOM | 50 | CA ILE | 7 | 25.098 | 27.943 | 34.547 | 1.00 | 11.49 | |
| ATOM | 51 | CB | ILE | 7 | 25.811 | 28.121 | 35.898 | 1.00 | 11.70 |
| ATOM | 52 | CG1 | ILE | 7 | 27.331 | 27.997 | 35.581 | 1.00 | 11.93 |
| ATOM | 53 | CD1 | ILE | 7 | 28.288 | 28.140 | 36.663 | 1.00 | 12.02 |
| ATOM | 54 | CG2 | ILE | 7 | 25.417 | 27.057 | 36.887 | 1.00 | 11.40 |
| ATOM | 55 | C | ILE | 7 | 23.634 | 27.664 | 34.603 | 1.00 | 11.29 |
| ATOM | 56 | O | ILE | 7 | 22.817 | 28.487 | 34.999 | 1.00 | 12.63 |
| ATOM | 57 | N | ILE | 8 | 23.245 | 26.433 | 34.193 | 1.00 | 10.48 |
| ATOM | 58 | CA | ILE | 8 | 21.856 | 26.026 | 34.213 | 1.00 | 9.51 |
| ATOM | 59 | CB | ILE | 8 | 21.513 | 24.909 | 33.253 | 1.00 | 8.83 |
| ATOM | 60 | CG1 | ILE | 8 | 22.221 | 23.575 | 33.487 | 1.00 | 7.90 |
| ATOM | 61 | CD1 | ILE | 8 | 21.762 | 22.525 | 32.454 | 1.00 | 7.81 |
| ATOM | 62 | CG2 | ILE | 8 | 21.684 | 25.377 | 31.803 | 1.00 | 9.89 |
| ATOM | 63 | C | ILE | 8 | 21.433 | 25.703 | 35.643 | 1.00 | 10.40 |
| ATOM | 64 | O | ILE | 8 | 22.205 | 25.174 | 36.456 | 1.00 | 9.94 |
| ATOM | 65 | N | ARG | 9 | 20.182 | 26.031 | 35.987 | 1.00 | 11.42 |
| ATOM | 66 | CA | ARG | 9 | 19.665 | 25.850 | 37.329 | 1.00 | 12.02 |
| ATOM | 67 | CB | ARG | 9 | 19.009 | 27.157 | 37.828 | 1.00 | 10.73 |
| ATOM | 68 | CG | ARG | 9 | 19.850 | 28.421 | 37.618 | 1.00 | 11.22 |
| ATOM | 69 | CD | ARG | 9 | 21.253 | 28.290 | 38.166 | 1.00 | 11.70 |
| ATOM | 70 | NE | ARG | 9 | 22.124 | 29.328 | 37.705 | 1.00 | 14.84 |
| ATOM | 71 | CZ | ARG | 9 | 22.235 | 30.599 | 38.010 | 1.00 | 15.66 |
| ATOM | 72 | NH1 | ARG | 9 | 21.529 | 31.122 | 38.991 | 1.00 | 18.52 |
| ATOM | 73 | NH2 | ARG | 9 | 22.902 | 31.393 | 37.188 | 1.00 | 14.81 |
| ATOM | 74 | C | ARG | 9 | 18.764 | 24.658 | 37.504 | 1.00 | 12.31 |
| ATOM | 75 | O | ARG | 9 | 18.267 | 24.036 | 36.555 | 1.00 | 14.42 |
| ATOM | 76 | N | ASP | 10 | 18.518 | 24.283 | 38.736 | 1.00 | 12.74 |
| ATOM | 77 | CA | ASP | 10 | 17.674 | 23.150 | 39.113 | 1.00 | 13.72 |
| ATOM | 78 | CB | ASP | 10 | 17.681 | 22.953 | 40.600 | 1.00 | 14.31 |
| ATOM | 79 | CG | ASP | 10 | 16.924 | 21.758 | 41.104 | 1.00 | 14.46 |
| ATOM | 80 | OD1 | ASP | 10 | 17.107 | 20.628 | 40.640 | 1.00 | 14.86 |
| ATOM | 81 | OD2 | ASP | 10 | 16.146 | 21.966 | 42.029 | 1.00 | 16.72 |
| ATOM | 82 | C | ASP | 10 | 16.285 | 23.236 | 38.506 | 1.00 | 14.81 |
| ATOM | 83 | O | ASP | 10 | 15.531 | 24.212 | 38.663 | 1.00 | 15.66 |
| ATOM | 84 | N | GLY | 11 | 15.932 | 22.174 | 37.772 | 1.00 | 14.39 |
| ATOM | 85 | CA | GLY | 11 | 14.636 | 22.169 | 37.079 | 1.00 | 15.06 |
| ATOM | 86 | C | GLY | 11 | 14.962 | 22.038 | 35.578 | 1.00 | 15.47 |
| ATOM | 87 | O | GLY | 11 | 14.116 | 21.564 | 34.841 | 1.00 | 16.98 |
| ATOM | 88 | N | HIS | 12 | 16.200 | 22.335 | 35.197 | 1.00 | 16.07 |
| ATOM | 89 | CA | HIS | 12 | 16.564 | 22.199 | 33.771 | 1.00 | 15.33 |
| ATOM | 90 | CB | HIS | 12 | 17.798 | 22.984 | 33.422 | 1.00 | 14.11 |
| ATOM | 91 | CG | HIS | 12 | 18.137 | 23.113 | 31.958 | 1.00 | 12.04 |
| ATOM | 92 | ND1 | HIS | 12 | 18.258 | 22.085 | 31.076 | 1.00 | 10.56 |
| ATOM | 93 | CE1 | HIS | 12 | 18.600 | 22.523 | 29.883 | 1.00 | 9.61 |
| ATOM | 94 | NE2 | HIS | 12 | 18.767 | 23.826 | 29.977 | 1.00 | 11.37 |
| ATOM | 95 | CD2 | HIS | 12 | 18.457 | 24.243 | 31.262 | 1.00 | 11.65 |
| ATOM | 96 | C | HIS | 12 | 16.780 | 20.692 | 33.515 | 1.00 | 15.36 |
| ATOM | 97 | O | HIS | 12 | 17.443 | 19.998 | 34.299 | 1.00 | 14.84 |
| ATOM | 98 | N | PRO | 13 | 16.209 | 20.178 | 32.431 | 1.00 | 14.53 |
| ATOM | 99 | CA | PRO | 13 | 16.296 | 18.798 | 32.066 | 1.00 | 14.20 |
| ATOM | 100 | CB | PRO | 13 | 15.436 | 18.664 | 30.843 | 1.00 | 14.95 |
| ATOM | 101 | CG | PRO | 13 | 15.070 | 20.047 | 30.408 | 1.00 | 15.00 |
| ATOM | 102 | CD | PRO | 13 | 15.333 | 20.975 | 31.520 | 1.00 | 14.75 |
| ATOM | 103 | C | PRO | 13 | 17.704 | 18.221 | 31.869 | 1.00 | 13.44 |
| ATOM | 104 | O | PRO | 13 | 17.920 | 17.049 | 32.297 | 1.00 | 13.01 |
| ATOM | 105 | N | THR | 14 | 18.641 | 18.885 | 31.313 | 1.00 | 12.25 |
| ATOM | 106 | CA | THR | 14 | 20.006 | 18.412 | 31.110 | 1.00 | 12.11 |
| ATOM | 107 | CB | THR | 14 | 20.879 | 19.447 | 30.442 | 1.00 | 12.39 |
| ATOM | 108 | OG1 | THR | 14 | 20.324 | 19.783 | 29.186 | 1.00 | 14.02 |
| ATOM | 109 | CG2 | THR | 14 | 22.327 | 19.047 | 30.251 | 1.00 | 11.43 |
| ATOM | 110 | C | THR | 14 | 20.616 | 17.961 | 32.443 | 1.00 | 11.87 |
| ATOM | 111 | O | THR | 14 | 21.340 | 16.974 | 32.439 | 1.00 | 12.53 |
| ATOM | 112 | N | LEU | 15 | 20.236 | 18.589 | 33.544 | 1.00 | 11.35 |
| ATOM | 113 | CA | LEU | 15 | 20.691 | 18.204 | 34.862 | 1.00 | 10.88 |
| ATOM | 114 | CB | LEU | 15 | 20.289 | 19.242 | 35.945 | 1.00 | 9.29 |
| ATOM | 115 | CG | LEU | 15 | 20.941 | 20.618 | 35.797 | 1.00 | 10.23 |
| ATOM | 116 | CD1 | LEU | 15 | 20.421 | 21.579 | 36.861 | 1.00 | 9.66 |
| ATOM | 117 | CD2 | LEU | 15 | 22.486 | 20.500 | 35.940 | 1.00 | 8.87 |
| ATOM | 118 | C | LEU | 15 | 20.276 | 16.823 | 35.350 | 1.00 | 10.52 |
| ATOM | 119 | O | LEU | 15 | 20.887 | 16.276 | 36.276 | 1.00 | 10.57 |
| ATOM | 120 | N | ARG | 16 | 19.281 | 16.212 | 34.728 | 1.00 | 12.62 |
| ATOM | 121 | CA | ARG | 16 | 18.760 | 14.908 | 35.102 | 1.00 | 12.70 |
| ATOM | 122 | CB | ARG | 16 | 17.252 | 15.021 | 35.435 | 1.00 | 11.19 |
| ATOM | 123 | CG | ARG | 16 | 16.965 | 15.901 | 36.689 | 1.00 | 12.05 |
| ATOM | 124 | CD | ARG | 16 | 17.589 | 15.300 | 37.922 | 1.00 | 12.29 |
| ATOM | 125 | NE | ARG | 16 | 17.174 | 15.869 | 39.202 | 1.00 | 14.07 |
| ATOM | 126 | CZ | ARG | 16 | 17.503 | 15.282 | 40.354 | 1.00 | 14.85 |
| ATOM | 127 | NH1 | ARG | 16 | 18.257 | 14.175 | 40.357 | 1.00 | 13.47 |
| ATOM | 128 | NH2 | ARG | 16 | 17.016 | 15.724 | 41.537 | 1.00 | 14.89 |
| ATOM | 129 | C | ARG | 16 | 19.050 | 13.808 | 34.098 | 1.00 | 13.29 |
| ATOM | 130 | O | ARG | 16 | 18.686 | 12.615 | 34.267 | 1.00 | 12.21 |
| ATOM | 131 | N | GLN | 17 | 19.716 | 14.156 | 33.007 | 1.00 | 14.43 |
| ATOM | 132 | CA | GLN | 17 | 20.112 | 13.203 | 31.993 | 1.00 | 14.49 |
| ATOM | 133 | CB | GLN | 17 | 20.423 | 13.917 | 30.676 | 1.00 | 15.50 |
| ATOM | 134 | CG | GLN | 17 | 19.172 | 14.623 | 30.150 | 1.00 | 19.61 |
| ATOM | 135 | CD | CLN | 17 | 19.464 | 15.367 | 28.883 | 1.00 | 22.76 |
| ATOM | 136 | OE1 | GLN | 17 | 20.585 | 15.845 | 28.654 | 1.00 | 25.94 |
| ATOM | 137 | NE2 | GLN | 17 | 18.516 | 15.484 | 27.983 | 1.00 | 25.81 |
| ATOM | 138 | C | GLN | 17 | 21.414 | 12.531 | 32.438 | 1.00 | 14.27 |
| ATOM | 139 | O | GLN | 17 | 22.117 | 12.968 | 33.350 | 1.00 | 13.89 |
| ATOM | 140 | N | LYS | 18 | 21.716 | 11.457 | 31.735 | 1.00 | 14.65 |
| ATOM | 141 | CA | LYS | 18 | 22.963 | 10.724 | 31.984 | 1.00 | 13.90 |
| ATOM | 142 | CB | LYS | 18 | 22.734 | 9.222 | 32.030 | 1.00 | 15.80 |
| ATOM | 143 | CG | LYS | 18 | 24.083 | 8.533 | 32.321 | 1.00 | 19.05 |
| ATOM | 144 | CD | LYS | 18 | 23.986 | 7.048 | 32.414 | 1.00 | 21.35 |
| ATOM | 145 | CE | LYS | 18 | 25.337 | 6.413 | 32.686 | 1.00 | 22.45 |
| ATOM | 146 | NZ | LYS | 18 | 25.078 | 4.922 | 32.839 | 1.00 | 26.17 |
| ATOM | 147 | C | LYS | 18 | 23.980 | 11.178 | 30.950 | 1.00 | 12.55 |
| ATOM | 148 | O | LYS | 18 | 23.801 | 10.968 | 29.753 | 1.00 | 14.36 |
| ATOM | 149 | N | ALA | 19 | 25.068 | 11.839 | 31.353 | 1.00 | 10.71 |
| ATOM | 150 | CA | ALA | 19 | 26.065 | 12.355 | 30.478 | 1.00 | 10.46 |
| ATOM | 151 | CB | ALA | 19 | 27.088 | 13.257 | 31.241 | 1.00 | 9.78 |
| ATOM | 152 | C | ALA | 19 | 26.749 | 11.295 | 29.636 | 1.00 | 11.00 |
| ATOM | 153 | O | ALA | 19 | 26.983 | 10.143 | 30.034 | 1.00 | 12.01 |
| ATOM | 154 | N | ALA | 20 | 27.080 | 11.667 | 28.404 | 1.00 | 10.99 |
| ATOM | 155 | CA | ALA | 20 | 27.720 | 10.828 | 27.437 | 1.00 | 9.79 |
| ATOM | 156 | CB | ALA | 20 | 27.497 | 11.492 | 26.021 | 1.00 | 8.56 |
| ATOM | 157 | C | ALA | 20 | 29.232 | 10.755 | 27.581 | 1.00 | 9.39 |
| ATOM | 158 | O | ALA | 20 | 29.876 | 11.797 | 27.803 | 1.00 | 8.57 |
| ATOM | 159 | N | GLU | 21 | 29.758 | 9.569 | 27.411 | 1.00 | 9.74 |
| ATOM | 160 | CA | GLU | 21 | 31.202 | 9.379 | 27.439 | 1.00 | 11.72 |
| ATOM | 161 | CB | GLU | 21 | 31.512 | 7.905 | 27.257 | 1.00 | 17.23 |
| ATOM | 162 | CG | GLU | 21 | 31.202 | 6.871 | 28.231 | 1.00 | 22.07 |
| ATOM | 163 | CD | GLU | 21 | 32.063 | 6.663 | 29.432 | 1.00 | 26.07 |
| ATOM | 164 | OE1 | GLU | 21 | 33.305 | 6.831 | 29.247 | 1.00 | 28.08 |
| ATOM | 165 | OE2 | GLU | 21 | 31.599 | 6.155 | 30.497 | 1.00 | 27.56 |
| ATOM | 166 | C | GLU | 21 | 31.861 | 10.115 | 26.238 | 1.00 | 11.80 |
| ATOM | 167 | O | GLU | 21 | 31.417 | 10.185 | 25.089 | 1.00 | 11.59 |
| ATOM | 168 | N | LEU | 22 | 33.013 | 10.661 | 26.528 | 1.00 | 11.07 |
| ATOM | 169 | CA | LEU | 22 | 33.838 | 11.349 | 25.587 | 1.00 | 11.81 |
| ATOM | 170 | CB | LEU | 22 | 34.811 | 12.325 | 26.236 | 1.00 | 9.69 |
| ATOM | 171 | CG | LEU | 22 | 34.424 | 13.644 | 26.794 | 1.00 | 8.78 |
| ATOM | 172 | CD1 | LEU | 22 | 34.103 | 14.677 | 25.719 | 1.00 | 8.17 |
| ATOM | 173 | CD2 | LEU | 22 | 33.310 | 13.559 | 27.815 | 1.00 | 8.71 |
| ATOM | 174 | C | LEU | 22 | 34.675 | 10.321 | 24.815 | 1.00 | 13.30 |
| ATOM | 175 | O | LEU | 22 | 35.079 | 9.332 | 25.372 | 1.00 | 13.83 |
| ATOM | 176 | N | GLU | 23 | 34.852 | 10.597 | 23.535 | 1.00 | 16.09 |
| ATOM | 177 | CA | GLU | 23 | 35.751 | 9.770 | 22.737 | 1.00 | 17.85 |
| ATOM | 178 | CB | GLU | 23 | 35.257 | 9.636 | 21.289 | 1.00 | 23.31 |
| ATOM | 179 | CG | GLU | 23 | 33.952 | 8.895 | 21.273 | 1.00 | 29.61 |
| ATOM | 180 | CD | GLU | 23 | 33.367 | 8.488 | 19.961 | 1.00 | 34.86 |
| ATOM | 181 | OE1 | GLU | 23 | 33.510 | 9.170 | 18.922 | 1.00 | 37.14 |
| ATOM | 182 | OE2 | GLU | 23 | 32.683 | 7.419 | 19.974 | 1.00 | 37.69 |
| ATOM | 183 | C | GLU | 23 | 37.087 | 10.532 | 22.787 | 1.00 | 16.82 |
| ATOM | 184 | O | GLU | 23 | 37.021 | 11.729 | 22.817 | 1.00 | 16.18 |
| ATOM | 185 | N | LEU | 24 | 38.209 | 9.855 | 22.917 | 1.00 | 18.03 |
| ATOM | 186 | CA | LEU | 24 | 39.524 | 10.444 | 22.930 | 1.00 | 17.31 |
| ATOM | 187 | CB | LEU | 24 | 40.436 | 9.892 | 24.031 | 1.00 | 16.16 |
| ATOM | 188 | CG | LEU | 24 | 40.230 | 10.228 | 25.490 | 1.00 | 16.48 |
| ATOM | 189 | CD1 | LEU | 24 | 40.257 | 11.765 | 25.662 | 1.00 | 17.54 |
| ATOM | 190 | CD2 | LEU | 24 | 38.965 | 9.743 | 26.117 | 1.00 | 15.61 |
| ATOM | 191 | C | LEU | 24 | 40.173 | 10.220 | 21.558 | 1.00 | 17.41 |
| ATOM | 192 | O | LEU | 24 | 39.995 | 9.166 | 20.916 | 1.00 | 19.08 |
| ATOM | 193 | N | PRO | 25 | 40.912 | 11.204 | 21.075 | 1.00 | 15.93 |
| ATOM | 194 | CA | PRO | 25 | 41.141 | 12.448 | 21.731 | 1.00 | 14.52 |
| ATOM | 195 | CB | PRO | 25 | 42.287 | 13.063 | 20.900 | 1.00 | 14.29 |
| ATOM | 196 | CG | PRO | 25 | 42.036 | 12.531 | 19.526 | 1.00 | 15.30 |
| ATOM | 197 | CD | PRO | 25 | 41.604 | 11.109 | 19.738 | 1.00 | 15.06 |
| ATOM | 198 | C | PRO | 25 | 39.995 | 13.443 | 21.653 | 1.00 | 13.35 |
| ATOM | 199 | O | PRO | 25 | 39.177 | 13.401 | 20.754 | 1.00 | 13.89 |
| ATOM | 200 | N | LEU | 26 | 39.985 | 14.367 | 22.602 | 1.00 | 13.79 |
| ATOM | 201 | CA | LEU | 26 | 38.996 | 15.418 | 22.666 | 1.00 | 12.59 |
| ATOM | 202 | CB | LEU | 26 | 39.082 | 16.256 | 23.904 | 1.00 | 12.08 |
| ATOM | 203 | CG | LEU | 26 | 38.886 | 15.669 | 25.289 | 1.00 | 12.93 |
| ATOM | 204 | CD1 | LEU | 26 | 38.866 | 16.788 | 26.340 | 1.00 | 10.60 |
| ATOM | 205 | CD2 | LEU | 26 | 37.642 | 14.803 | 25.381 | 1.00 | 12.07 |
| ATOM | 206 | C | LEU | 26 | 39.100 | 16.326 | 21.409 | 1.00 | 12.36 |
| ATOM | 207 | O | LEU | 26 | 40.197 | 16.480 | 20.886 | 1.00 | 12.03 |
| ATOM | 208 | N | THR | 27 | 37.963 | 16.834 | 20.967 | 1.00 | 12.65 |
| ATOM | 209 | CA | THR | 27 | 37.963 | 17.766 | 19.832 | 1.00 | 14.54 |
| ATOM | 210 | CB | THR | 27 | 36.532 | 18.035 | 19.308 | 1.00 | 15.30 |
| ATOM | 211 | OG1 | THR | 27 | 35.727 | 18.682 | 20.310 | 1.00 | 15.81 |
| ATOM | 212 | CG2 | THR | 27 | 35.847 | 16.719 | 18.948 | 1.00 | 14.42 |
| ATOM | 213 | C | THR | 27 | 38.493 | 19.093 | 20.391 | 1.00 | 15.69 |
| ATOM | 214 | O | THR | 27 | 38.511 | 19.249 | 21.642 | 1.00 | 15.10 |
| ATOM | 215 | N | LYS | 28 | 38.949 | 20.026 | 19.593 | 1.00 | 17.11 |
| ATOM | 216 | CA | LYS | 28 | 39.461 | 21.322 | 20.062 | 1.00 | 16.59 |
| ATOM | 217 | CB | LYS | 28 | 39.640 | 22.170 | 18.785 | 1.00 | 21.45 |
| ATOM | 218 | CG | LYS | 28 | 40.041 | 23.601 | 18.941 | 1.00 | 24.97 |
| ATOM | 219 | CD | LYS | 28 | 41.424 | 23.746 | 19.542 | 1.00 | 28.58 |
| ATOM | 220 | CE | LYS | 28 | 41.893 | 25.200 | 19.520 | 1.00 | 29.78 |
| ATOM | 221 | NZ | LYS | 28 | 43.362 | 25.231 | 19.863 | 1.00 | 30.20 |
| ATOM | 222 | C | LYS | 28 | 38.449 | 22.070 | 20.922 | 1.00 | 15.27 |
| ATOM | 223 | O | LYS | 28 | 38.795 | 22.754 | 21.899 | 1.00 | 14.45 |
| ATOM | 224 | N | GLU | 29 | 37.202 | 21.988 | 20.547 | 1.00 | 14.88 |
| ATOM | 225 | CA | GLU | 29 | 36.095 | 22.628 | 21.221 | 1.00 | 14.47 |
| ATOM | 226 | CB | GLU | 29 | 34.832 | 22.548 | 20.375 | 1.00 | 20.18 |
| ATOM | 227 | CG | GLO | 29 | 33.633 | 23.176 | 21.057 | 1.00 | 25.80 |
| ATOM | 228 | CD | GLU | 29 | 32.326 | 22.956 | 20.331 | 1.00 | 30.91 |
| ATOM | 229 | OE1 | GLU | 29 | 32.201 | 21.989 | 19.520 | 1.00 | 32.53 |
| ATOM | 230 | OE2 | GLU | 29 | 31.389 | 23.743 | 20.643 | 1.00 | 32.71 |
| ATOM | 231 | C | GLU | 29 | 35.873 | 22.024 | 22.595 | 1.00 | 13.31 |
| ATOM | 232 | O | GLU | 29 | 35.595 | 22.749 | 23.545 | 1.00 | 11.60 |
| ATOM | 233 | N | GLU | 30 | 35.940 | 20.686 | 22.711 | 1.00 | 12.75 |
| ATOM | 234 | CA | GLU | 30 | 35.795 | 20.032 | 24.009 | 1.00 | 10.36 |
| ATOM | 235 | CB | GLU | 30 | 35.700 | 18.517 | 23.814 | 1.00 | 10.77 |
| ATOM | 236 | CG | GLU | 30 | 34.365 | 18.110 | 23.139 | 1.00 | 12.50 |
| ATOM | 237 | CD | GLU | 30 | 34.397 | 16.669 | 22.642 | 1.00 | 11.90 |
| ATOM | 238 | OE1 | GLU | 30 | 35.467 | 16.104 | 22.370 | 1.00 | 12.79 |
| ATOM | 239 | OE2 | GLU | 30 | 33.310 | 16.151 | 22.474 | 1.00 | 14.28 |
| ATOM | 240 | C | GLU | 30 | 36.936 | 20.426 | 24.950 | 1.00 | 9.86 |
| ATOM | 241 | O | GLU | 30 | 36.678 | 20.595 | 26.156 | 1.00 | 9.16 |
| ATOM | 242 | N | LYS | 31 | 38.154 | 20.617 | 24.466 | 1.00 | 9.50 |
| ATOM | 243 | CA | LYS | 31 | 39.272 | 21.016 | 25.315 | 1.00 | 10.37 |
| ATOM | 244 | CB | LYS | 31 | 40.624 | 20.903 | 24.615 | 1.00 | 10.65 |
| ATOM | 245 | CG | LYS | 31 | 40.935 | 19.492 | 24.109 | 1.00 | 10.14 |
| ATOM | 246 | CD | LYS | 31 | 42.376 | 19.413 | 23.639 | 1.00 | 12.65 |
| ATOM | 247 | CE | LYS | 31 | 42.773 | 18.053 | 23.142 | 1.00 | 13.40 |
| ATOM | 248 | NZ | LYS | 31 | 44.122 | 17.978 | 22.539 | 1.00 | 16.75 |
| ATOM | 249 | C | LYS | 31 | 39.056 | 22.437 | 25.839 | 1.00 | 11.34 |
| ATOM | 250 | O | LYS | 31 | 39.219 | 22.729 | 27.019 | 1.00 | 10.78 |
| ATOM | 251 | N | GLU | 32 | 38.652 | 23.343 | 24.963 | 1.00 | 12.34 |
| ATOM | 252 | CA | GLU | 32 | 38.358 | 24.725 | 25.267 | 1.00 | 13.73 |
| ATOM | 253 | CB | GLU | 32 | 37.914 | 25.458 | 23.995 | 1.00 | 18.88 |
| ATOM | 254 | CG | GLU | 32 | 38.998 | 25.596 | 22.948 | 1.00 | 25.82 |
| ATOM | 255 | CD | GLU | 32 | 38.508 | 26.242 | 21.661 | 1.00 | 31.02 |
| ATOM | 256 | OE1 | GLU | 32 | 37.329 | 26.619 | 21.506 | 1.00 | 34.16 |
| ATOM | 257 | OE2 | GLU | 32 | 39.333 | 26.362 | 20.729 | 1.00 | 33.69 |
| ATOM | 258 | C | GLU | 32 | 37.266 | 24.829 | 26.333 | 1.00 | 12.41 |
| ATOM | 259 | O | GLU | 32 | 37.312 | 25.639 | 27.241 | 1.00 | 11.77 |
| ATOM | 260 | N | THR | 33 | 36.250 | 23.978 | 26.208 | 1.00 | 12.31 |
| ATOM | 261 | CA | THR | 33 | 35.163 | 23.899 | 27.157 | 1.00 | 12.37 |
| ATOM | 262 | CB | THR | 33 | 34.104 | 22.861 | 26.725 | 1.00 | 13.11 |
| ATOM | 263 | CD1 | THR | 33 | 33.517 | 23.349 | 25.513 | 1.00 | 15.23 |
| ATOM | 264 | CD2 | THR | 33 | 33.023 | 22.657 | 27.752 | 1.00 | 11.29 |
| ATOM | 265 | C | THR | 33 | 35.681 | 23.524 | 28.559 | 1.00 | 11.41 |
| ATOM | 266 | O | THR | 33 | 35.365 | 24.208 | 29.497 | 1.00 | 10.34 |
| ATOM | 267 | N | LEU | 34 | 36.486 | 22.474 | 28.658 | 1.00 | 11.17 |
| ATOM | 268 | CA | LEU | 34 | 37.030 | 22.021 | 29.928 | 1.00 | 10.16 |
| ATOM | 269 | CB | LEU | 34 | 37.678 | 20.654 | 29.693 | 1.00 | 9.41 |
| ATOM | 270 | CG | LEU | 34 | 38.102 | 19.874 | 30.942 | 1.00 | 8.70 |
| ATOM | 271 | CD1 | LEU | 34 | 36.909 | 19.607 | 31.858 | 1.00 | 5.18 |
| ATOM | 272 | CD2 | LEU | 34 | 38.697 | 18.541 | 30.452 | 1.00 | 8.15 |
| ATOM | 273 | C | LEU | 34 | 37.960 | 23.036 | 30.557 | 1.00 | 10.05 |
| ATOM | 274 | O | LEU | 34 | 37.967 | 23.258 | 31.785 | 1.00 | 10.33 |
| ATOM | 275 | N | ILE | 35 | 38.761 | 23.700 | 29.745 | 1.00 | 9.38 |
| ATOM | 276 | CA | ILE | 35 | 39.669 | 24.770 | 30.298 | 1.00 | 9.27 |
| ATOM | 277 | CB | ILE | 35 | 40.619 | 25.168 | 29.153 | 1.00 | 9.96 |
| ATOM | 278 | CG1 | ILE | 35 | 41.392 | 23.960 | 28.683 | 1.00 | 11.21 |
| ATOM | 279 | CD1 | ILE | 35 | 42.276 | 24.096 | 27.476 | 1.00 | 10.72 |
| ATOM | 280 | CG2 | ILE | 35 | 41.467 | 26.360 | 29.493 | 1.00 | 10.81 |
| ATOM | 281 | C | ILE | 35 | 38.843 | 25.908 | 30.801 | 1.00 | 8.81 |
| ATOM | 282 | O | ILE | 35 | 39.131 | 26.555 | 31.836 | 1.00 | 9.47 |
| ATOM | 283 | N | ALA | 36 | 37.769 | 26.277 | 30.047 | 1.00 | 8.60 |
| ATOM | 284 | CA | ALA | 36 | 36.878 | 27.369 | 30.469 | 1.00 | 8.07 |
| ATOM | 285 | CB | ALA | 36 | 35.833 | 27.665 | 29.390 | 1.00 | 8.37 |
| ATOM | 286 | C | ALA | 36 | 36.161 | 27.045 | 31.767 | 1.00 | 8.14 |
| ATOM | 287 | O | ALA | 36 | 35.768 | 27.917 | 32.560 | 1.00 | 8.12 |
| ATOM | 288 | N | MET | 37 | 35.881 | 25.724 | 31.941 | 1.00 | 8.77 |
| ATOM | 289 | CA | MET | 37 | 35.248 | 25.210 | 33.136 | 1.00 | 9.24 |
| ATOM | 290 | CB | MET | 37 | 34.795 | 23.764 | 33.059 | 1.00 | 8.72 |
| ATOM | 291 | CG | MET | 37 | 33.559 | 23.483 | 32.224 | 1.00 | 6.71 |
| ATOM | 292 | SD | MET | 37 | 33.335 | 21.789 | 31.709 | 1.00 | 6.53 |
| ATOM | 293 | CE | MET | 37 | 33.342 | 20.932 | 33.251 | 1.00 | 4.67 |
| ATOM | 294 | C | MET | 37 | 36.195 | 25.410 | 34.333 | 1.00 | 9.17 |
| ATOM | 295 | O | MET | 37 | 35.692 | 25.892 | 35.376 | 1.00 | 8.49 |
| ATOM | 296 | N | ARG | 38 | 37.452 | 25.050 | 34.165 | 1.00 | 8.88 |
| ATOM | 297 | CA | ARG | 38 | 38.401 | 25.295 | 35.266 | 1.00 | 8.29 |
| ATOM | 298 | CB | ARG | 38 | 39.761 | 24.673 | 34.946 | 1.00 | 6.27 |
| ATOM | 299 | CG | ARG | 38 | 40.886 | 25.129 | 35.854 | 1.00 | 6.22 |
| ATOM | 300 | CD | ARG | 38 | 42.253 | 24.472 | 35.513 | 1.00 | 6.20 |
| ATOM | 301 | NE | ARG | 38 | 43.275 | 24.989 | 36.431 | 1.00 | 8.87 |
| ATOM | 302 | CZ | ARG | 38 | 43.890 | 26.160 | 36.395 | 1.00 | 10.20 |
| ATOM | 303 | NH1 | ARG | 38 | 43.647 | 27.040 | 35.409 | 1.00 | 11.24 |
| ATOM | 304 | NH2 | ARG | 38 | 44.784 | 26.493 | 37.315 | 1.00 | 11.77 |
| ATOM | 305 | C | ARG | 38 | 38.583 | 26.825 | 35.430 | 1.00 | 9.07 |
| ATOM | 306 | O | ARG | 38 | 38.763 | 27.261 | 36.567 | 1.00 | 9.36 |
| ATOM | 307 | N | GLU | 39 | 38.575 | 27.572 | 34.337 | 1.00 | 8.69 |
| ATOM | 308 | CA | GLU | 39 | 38.771 | 29.029 | 34.403 | 1.00 | 9.54 |
| ATOM | 309 | CB | GLU | 39 | 39.029 | 29.643 | 33.049 | 1.00 | 10.78 |
| ATOM | 310 | CG | GLU | 39 | 39.650 | 31.040 | 33.051 | 1.00 | 14.31 |
| ATOM | 311 | CD | CLU | 39 | 41.070 | 30.997 | 33.632 | 1.00 | 17.29 |
| ATOM | 312 | OE1 | GLU | 39 | 41.712 | 29.936 | 33.706 | 1.00 | 17.09 |
| ATOM | 313 | OE2 | GLU | 39 | 41.577 | 32.041 | 34.079 | 1.00 | 19.58 |
| ATOM | 314 | C | GLU | 39 | 37.629 | 29.691 | 35.178 | 1.00 | 8.74 |
| ATOM | 315 | O | GLU | 39 | 37.872 | 30.680 | 35.887 | 1.00 | 8.33 |
| ATOM | 316 | N | PHE | 40 | 36.424 | 29.202 | 35.067 | 1.00 | 8.68 |
| ATOM | 317 | CA | PHE | 40 | 35.267 | 29.674 | 35.808 | 1.00 | 8.98 |
| ATOM | 318 | CB | PHE | 40 | 33.969 | 28.890 | 35.546 | 1.00 | 6.33 |
| ATOM | 319 | CG | PHE | 40 | 32.816 | 29.364 | 36.423 | 1.00 | 7.37 |
| ATOM | 320 | CD1 | PHE | 40 | 32.004 | 30.431 | 36.041 | 1.00 | 6.34 |
| ATOM | 321 | CE1 | PHE | 40 | 30.944 | 30.806 | 36.826 | 1.00 | 7.39 |
| ATOM | 322 | CZ | PHE | 40 | 30.729 | 30.221 | 38.073 | 1.00 | 8.75 |
| ATOM | 323 | CE2 | PHE | 40 | 31.553 | 29.164 | 38.474 | 1.00 | 7.42 |
| ATOM | 324 | CD2 | PHE | 40 | 32.570 | 28.749 | 37.650 | 1.00 | 5.86 |
| ATOM | 325 | C | PHE | 40 | 35.567 | 29.539 | 37.321 | 1.00 | 9.68 |
| ATOM | 326 | O | PHE | 40 | 35.299 | 30.458 | 38.106 | 1.00 | 8.96 |
| ATOM | 327 | N | LEU | 41 | 36.096 | 28.389 | 37.716 | 1.00 | 8.34 |
| ATOM | 328 | CA | LEU | 41 | 36.407 | 28.121 | 39.108 | 1.00 | 7.20 |
| ATOM | 329 | CB | LEU | 41 | 36.736 | 26.662 | 39.311 | 1.00 | 5.79 |
| ATOM | 330 | CG | LEU | 41 | 35.632 | 25.661 | 38.986 | 1.00 | 4.96 |
| ATOM | 331 | CD1 | LEU | 41 | 36.180 | 24.230 | 39.076 | 1.00 | 4.68 |
| ATOM | 332 | CD2 | LEU | 41 | 34.592 | 25.769 | 40.118 | 1.00 | 5.67 |
| ATOM | 333 | C | LEU | 41 | 37.487 | 29.057 | 39.633 | 1.00 | 6.55 |
| ATOM | 334 | O | LEU | 41 | 37.318 | 29.593 | 40.732 | 1.00 | 6.21 |
| ATOM | 335 | N | VAL | 42 | 38.543 | 29.226 | 38.866 | 1.00 | 6.90 |
| ATOM | 336 | CA | VAL | 42 | 39.620 | 30.141 | 39.229 | 1.00 | 8.11 |
| ATOM | 337 | CB | VAL | 42 | 40.640 | 30.109 | 38.086 | 1.00 | 11.15 |
| ATOM | 338 | CG1 | VAL | 42 | 41.734 | 31.155 | 38.206 | 1.00 | 13.45 |
| ATOM | 339 | CG2 | VAL | 42 | 41.304 | 28.748 | 37.976 | 1.00 | 11.23 |
| ATOM | 340 | C | VAL | 42 | 38.991 | 31.513 | 39.408 | 1.00 | 8.71 |
| ATOM | 341 | O | VAL | 42 | 39.122 | 32.126 | 40.489 | 1.00 | 10.31 |
| ATOM | 342 | N | ASN | 43 | 38.252 | 32.049 | 38.442 | 1.00 | 8.34 |
| ATOM | 343 | CA | ASN | 43 | 37.596 | 33.354 | 38.565 | 1.00 | 8.46 |
| ATOM | 344 | CB | ASN | 43 | 36.811 | 33.707 | 37.239 | 1.00 | 8.52 |
| ATOM | 345 | CG | ASN | 43 | 37.814 | 34.020 | 36.161 | 1.00 | 9.24 |
| ATOM | 346 | OD1 | ASN | 43 | 38.937 | 34.339 | 36.546 | 1.00 | 10.81 |
| ATOM | 347 | ND2 | ASN | 43 | 37.464 | 33.943 | 34.891 | 1.00 | 10.73 |
| ATOM | 348 | C | ASN | 43 | 36.632 | 33.504 | 39.695 | 1.00 | 9.08 |
| ATOM | 349 | O | ASN | 43 | 36.559 | 34.588 | 40.314 | 1.00 | 8.98 |
| ATOM | 350 | N | SER | 44 | 35.887 | 32.473 | 40.035 | 1.00 | 9.07 |
| ATOM | 351 | CA | SER | 44 | 34.906 | 32.506 | 41.091 | 1.00 | 10.54 |
| ATOM | 352 | CB | SER | 44 | 33.940 | 31.317 | 41.107 | 1.00 | 9.94 |
| ATOM | 353 | OG | SER | 44 | 34.463 | 30.152 | 41.665 | 1.00 | 10.32 |
| ATOM | 354 | C | SER | 44 | 35.612 | 32.595 | 42.463 | 1.00 | 11.84 |
| ATOM | 355 | O | SER | 44 | 35.022 | 33.048 | 43.445 | 1.00 | 13.02 |
| ATOM | 356 | N | GLN | 45 | 36.866 | 32.177 | 42.517 | 1.00 | 12.29 |
| ATOM | 357 | CA | GLN | 45 | 37.648 | 32.199 | 43.727 | 1.00 | 12.76 |
| ATOM | 358 | CB | GLN | 45 | 38.552 | 30.951 | 43.870 | 1.00 | 13.51 |
| ATOM | 359 | CG | GLN | 45 | 37.711 | 29.688 | 44.112 | 1.00 | 12.21 |
| ATOM | 360 | CD | GLN | 45 | 38.518 | 28.436 | 44.161 | 1.00 | 13.51 |
| ATOM | 361 | OE1 | GLN | 45 | 39.733 | 28.479 | 44.331 | 1.00 | 15.12 |
| ATOM | 362 | NE2 | GLN | 45 | 37.819 | 27.301 | 43.898 | 1.00 | 11.45 |
| ATOM | 363 | C | GLN | 45 | 38.451 | 33.491 | 43.882 | 1.00 | 14.04 |
| ATOM | 364 | O | GLN | 45 | 38.996 | 33.746 | 44.942 | 1.00 | 14.63 |
| ATOM | 365 | N | ASP | 46 | 38.518 | 34.305 | 42.869 | 1.00 | 14.76 |
| ATOM | 366 | CA | ASP | 46 | 39.203 | 35.595 | 42.881 | 1.00 | 17.02 |
| ATOM | 367 | CB | ASP | 46 | 39.977 | 35.835 | 41.627 | 1.00 | 20.12 |
| ATOM | 368 | CD | ASP | 46 | 40.799 | 37.118 | 41.646 | 1.00 | 23.64 |
| ATOM | 369 | OD1 | ASP | 46 | 40.229 | 38.095 | 42.163 | 1.00 | 23.90 |
| ATOM | 370 | OD2 | ASP | 46 | 41.954 | 37.141 | 41.179 | 1.00 | 26.97 |
| ATOM | 371 | C | ASP | 46 | 38.159 | 36.639 | 43.278 | 1.00 | 16.96 |
| ATOM | 372 | O | ASP | 46 | 37.141 | 36.906 | 42.690 | 1.00 | 15.76 |
| ATOM | 373 | N | GLU | 47 | 38.383 | 37.201 | 44.471 | 1.00 | 18.61 |
| ATOM | 374 | CA | GLU | 47 | 37.524 | 38.172 | 45.105 | 1.00 | 20.77 |
| ATOM | 375 | CB | GLU | 47 | 38.318 | 38.747 | 46.281 | 1.00 | 26.35 |
| ATOM | 376 | CG | GLU | 47 | 37.601 | 39.794 | 47.124 | 1.00 | 31.95 |
| ATOM | 377 | CD | GLU | 47 | 38.607 | 40.361 | 48.151 | 1.00 | 35.24 |
| ATOM | 378 | OE1 | GLU | 47 | 39.590 | 41.011 | 47.707 | 1.00 | 36.36 |
| ATOM | 379 | OE2 | GLU | 47 | 38.401 | 40.085 | 49.347 | 1.00 | 37.08 |
| ATOM | 380 | C | GLU | 47 | 37.043 | 39.303 | 44.202 | 1.00 | 20.14 |
| ATOM | 381 | O | GLU | 47 | 35.844 | 39.612 | 44.172 | 1.00 | 19.26 |
| ATOM | 382 | N | GLU | 48 | 37.975 | 39.957 | 43.516 | 1.00 | 19.74 |
| ATOM | 383 | CA | GLU | 48 | 37.697 | 41.040 | 42.619 | 1.00 | 20.15 |
| ATOM | 384 | CB | GLU | 48 | 39.015 | 41.724 | 42.176 | 1.00 | 26.28 |
| ATOM | 385 | CG | GLU | 48 | 38.788 | 42.838 | 41.175 | 1.00 | 33.55 |
| ATOM | 386 | CD | GLU | 48 | 39.965 | 43.661 | 40.716 | 1.00 | 38.79 |
| ATOM | 387 | OE1 | CLU | 48 | 41.131 | 43.498 | 41.173 | 1.00 | 40.40 |
| ATOM | 388 | OE2 | GLU | 48 | 39.734 | 44.559 | 39.829 | 1.00 | 39.98 |
| ATOM | 389 | C | GLU | 48 | 36.940 | 40.613 | 41.365 | 1.00 | 17.56 |
| ATOM | 390 | O | GLU | 48 | 35.980 | 41.201 | 40.970 | 1.00 | 15.31 |
| ATOM | 391 | N | ILE | 49 | 37.483 | 39.560 | 40.714 | 1.00 | 17.33 |
| ATOM | 392 | CA | ILE | 49 | 36.899 | 39.040 | 39.494 | 1.00 | 15.12 |
| ATOM | 393 | CD | ILE | 49 | 37.765 | 38.038 | 38.757 | 1.00 | 16.13 |
| ATOM | 394 | CG1 | ILE | 49 | 39.111 | 38.674 | 38.292 | 1.00 | 16.15 |
| ATOM | 395 | CD1 | ILE | 49 | 40.129 | 37.587 | 37.857 | 1.00 | 13.47 |
| ATOM | 396 | CG2 | ILE | 49 | 36.997 | 37.575 | 37.496 | 1.00 | 16.11 |
| ATOM | 397 | C | ILE | 49 | 35.503 | 38.558 | 39.794 | 1.00 | 13.80 |
| ATOM | 398 | O | ILE | 49 | 34.619 | 38.936 | 39.034 | 1.00 | 14.67 |
| ATOM | 399 | N | ALA | 50 | 35.287 | 37.841 | 40.890 | 1.00 | 12.57 |
| ATOM | 400 | CA | ALA | 50 | 33.952 | 37.373 | 41.233 | 1.00 | 12.32 |
| ATOM | 401 | CB | ALA | 50 | 33.951 | 36.441 | 42.447 | 1.00 | 10.25 |
| ATOM | 402 | C | ALA | 50 | 33.017 | 38.526 | 41.500 | 1.00 | 12.90 |
| ATOM | 403 | O | ALA | 50 | 31.824 | 38.435 | 41.186 | 1.00 | 12.81 |
| ATOM | 404 | N | LYS | 51 | 33.530 | 39.604 | 42.123 | 1.00 | 14.84 |
| ATOM | 405 | CA | LYS | 51 | 32.621 | 40.724 | 42.364 | 1.00 | 17.61 |
| ATOM | 406 | CB | LYS | 51 | 33.059 | 41.746 | 43.393 | 1.00 | 23.09 |
| ATOM | 407 | CG | LYS | 51 | 33.488 | 41.245 | 44.742 | 1.00 | 29.47 |
| ATOM | 408 | CD | LYS | 51 | 32.742 | 40.030 | 45.276 | 1.00 | 35.11 |
| ATOM | 409 | CE | LYS | 51 | 31.236 | 40.116 | 45.393 | 1.00 | 38.34 |
| ATOM | 410 | NZ | LYS | 51 | 30.581 | 38.814 | 45.690 | 1.00 | 39.45 |
| ATOM | 411 | C | LYS | 51 | 32.262 | 41.424 | 41.035 | 1.00 | 16.20 |
| ATOM | 412 | O | LYS | 51 | 31.066 | 41.571 | 40.745 | 1.00 | 17.61 |
| ATOM | 413 | N | ARG | 52 | 33.240 | 41.788 | 40.263 | 1.00 | 15.36 |
| ATOM | 414 | CA | ARG | 52 | 33.029 | 42.487 | 38.995 | 1.00 | 15.36 |
| ATOM | 415 | CB | ARG | 52 | 34.299 | 42.597 | 38.210 | 1.00 | 15.92 |
| ATOM | 416 | CG | ARG | 52 | 34.190 | 43.493 | 36.961 | 1.00 | 20.56 |
| ATOM | 417 | CD | ARG 52 | 35.484 | 43.366 | 36.171 | 1.00 | 25.75 | |
| ATOM | 418 | NE | ARG | 52 | 36.584 | 43.664 | 37.113 | 1.00 | 30.94 |
| ATOM | 419 | CZ | ARG | 52 | 37.845 | 43.340 | 36.840 | 1.00 | 34.86 |
| ATOM | 420 | NH1 | ARG | 52 | 38.178 | 42.759 | 35.680 | 1.00 | 36.86 |
| ATOM | 421 | NH2 | ARG | 52 | 38.734 | 43.409 | 37.825 | 1.00 | 36.03 |
| ATOM | 422 | C | ARG | 52 | 32.016 | 41.733 | 38.124 | 1.00 | 15.87 |
| ATOM | 423 | O | ARG | 52 | 31.146 | 42.328 | 37.514 | 1.00 | 15.88 |
| ATOM | 424 | N | TYR | 53 | 32.184 | 40.394 | 38.051 | 1.00 | 15.45 |
| ATOM | 425 | CA | TYR | 53 | 31.314 | 39.566 | 37.273 | 1.00 | 14.70 |
| ATOM | 426 | CB | TYR | 53 | 32.073 | 38.518 | 36.434 | 1.00 | 15.59 |
| ATOM | 427 | CG | TYR | 53 | 32.954 | 39.236 | 35.446 | 1.00 | 16.81 |
| ATOM | 428 | CD1 | TYR | 53 | 32.407 | 39.794 | 34.291 | 1.00 | 17.94 |
| ATOM | 429 | CE1 | TYR | 53 | 33.218 | 40.509 | 33.416 | 1.00 | 19.07 |
| ATOM | 430 | CZ | TYR | 53 | 34.568 | 40.622 | 33.684 | 1.00 | 19.48 |
| ATOM | 431 | OH | TYR | 53 | 35.396 | 41.309 | 32.827 | 1.00 | 22.92 |
| ATOM | 432 | CE2 | TYR | 53 | 35.110 | 40.078 | 34.818 | 1.00 | 19.01 |
| ATOM | 433 | CD2 | TYR | 53 | 34.301 | 39.379 | 35.692 | 1.00 | 18.15 |
| ATOM | 434 | C | TYR | 53 | 30.181 | 38.907 | 37.974 | 1.00 | 14.36 |
| ATOM | 435 | O | TYR | 53 | 29.510 | 38.141 | 37.274 | 1.00 | 15.28 |
| ATOM | 436 | N | GLY | 54 | 29.926 | 39.126 | 39.252 | 1.00 | 14.71 |
| ATOM | 437 | CA GLY | 54 | 28.814 | 38.492 | 39.937 | 1.00 | 15.24 | |
| ATOM | 438 | C | GLY | 54 | 28.886 | 36.981 | 39.985 | 1.00 | 16.54 |
| ATOM | 439 | O | GLY | 54 | 27.879 | 36.281 | 39.822 | 1.00 | 19.66 |
| ATOM | 440 | N | LEU | 55 | 30.071 | 36.433 | 40.235 | 1.00 | 15.81 |
| ATOM | 441 | CA | LEU | 55 | 30.300 | 35.00 | 40.253 | 1.00 | 14.58 |
| ATOM | 442 | CB | LEU | 55 | 31.684 | 34.640 | 39.689 | 1.00 | 10.75 |
| ATOM | 443 | CG | LEU | 55 | 32.128 | 35.125 | 38.328 | 1.00 | 8.84 |
| ATOM | 444 | CD1 | LEU | 55 | 33.483 | 34.650 | 37.920 | 1.00 | 6.15 |
| ATOM | 445 | CD2 | LEU | 55 | 31.095 | 34.816 | 37.227 | 1.00 | 8.13 |
| ATOM | 446 | C | LEU | 55 | 30.165 | 34.407 | 41.645 | 1.00 | 14.82 |
| ATOM | 447 | O | LEU | 55 | 30.668 | 34.952 | 42.634 | 1.00 | 15.45 |
| ATOM | 448 | N | ARG | 56 | 29.397 | 33.312 | 41.702 | 1.00 | 13.86 |
| ATOM | 449 | CA | ARG | 56 | 29.28 | 32.583 | 42.994 | 1.00 | 14.05 |
| ATOM | 450 | CB | ARG | 56 | 27.988 | 31.773 | 42.901 | 1.00 | 12.84 |
| ATOM | 451 | CG | ARG | 56 | 27.702 | 30.880 | 44.040 | 1.00 | 12.65 |
| ATOM | 452 | CD | ARG | 56 | 26.371 | 30.171 | 43.928 | 1.00 | 13.01 |
| ATOM | 453 | NE | ARG | 56 | 26.264 | 29.199 | 44.998 | 1.00 | 13.72 |
| ATOM | 454 | CZ | ARG | 56 | 25.247 | 28.411 | 45.241 | 1.00 | 12.53 |
| ATOM | 455 | NH1 | ARG | 56 | 24.176 | 28.440 | 44.495 | 1.00 | 10.89 |
| ATOM | 456 | NH2 | ARG | 56 | 25.422 | 27.610 | 46.289 | 1.00 | 14.43 |
| ATOM | 457 | C | ARG | 56 | 30.501 | 31.671 | 43.133 | 1.00 | 14.45 |
| ATOM | 458 | O | ARG | 56 | 30.849 | 30.913 | 42.200 | 1.00 | 14.08 |
| ATOM | 459 | N | SER | 57 | 31.222 | 31.754 | 44.225 | 1.00 | 13.89 |
| ATOM | 460 | CA | SER | 57 | 32.426 | 30.929 | 44.412 | 1.00 | 15.05 |
| ATOM | 461 | CB | SER | 57 | 33.023 | 31.247 | 45.792 | 1.00 | 15.54 |
| ATOM | 462 | OG | SER | 57 | 34.277 | 30.604 | 45.995 | 1.00 | 17.22 |
| ATOM | 463 | C | SER | 57 | 32.107 | 29.424 | 44.362 | 1.00 | 14.39 |
| ATOM | 464 | O | SER | 57 | 31.028 | 29.003 | 44.817 | 1.00 | 14.42 |
| ATOM | 465 | N | GLY | 58 | 32.997 | 28.630 | 43.816 | 1.00 | 13.20 |
| ATOM | 466 | CA | GLY | 58 | 32.724 | 27.157 | 43.793 | 1.00 | 10.96 |
| ATOM | 467 | C | GLY | 58 | 34.050 | 26.420 | 43.697 | 1.00 | 8.84 |
| ATOM | 468 | O | GLY | 58 | 35.027 | 27.045 | 43.316 | 1.00 | 9.13 |
| ATOM | 469 | N | VAL | 59 | 34.082 | 25.126 | 43.982 | 1.00 | 8.47 |
| ATOM | 470 | CA | VAL | 59 | 35.220 | 24.274 | 43.887 | 1.00 | 7.38 |
| ATOM | 471 | CB | VAL | 59 | 35.731 | 23.702 | 45.248 | 1.00 | 6.40 |
| ATOM | 472 | CG1 | VAL | 59 | 36.137 | 24.837 | 46.154 | 1.00 | 2.67 |
| ATOM | 473 | CG2 | VAL | 59 | 34.729 | 22.751 | 45.834 | 1.00 | 6.06 |
| ATOM | 474 | C | VAL | 59 | 35.111 | 23.138 | 42.895 | 1.00 | 6.56 |
| ATOM | 475 | O | VAL | 59 | 36.087 | 22.423 | 42.623 | 1.00 | 5.81 |
| ATOM | 476 | N | GLY | 60 | 33.901 | 22.917 | 42.339 | 1.00 | 5.18 |
| ATOM | 477 | CA | GLY | 60 | 33.686 | 21.922 | 41.328 | 1.00 | 3.01 |
| ATOM | 478 | C | GLY | 60 | 32.684 | 22.363 | 40.281 | 1.00 | 4.83 |
| ATOM | 479 | O | GLY | 60 | 31.831 | 23.228 | 40.563 | 1.00 | 7.05 |
| ATOM | 480 | N | LEU | 61 | 32.723 | 21.833 | 39.071 | 1.00 | 4.97 |
| ATOM | 481 | CA | LEU | 61 | 31.800 | 22.185 | 38.012 | 1.00 | 5.34 |
| ATOM | 482 | CB | LEU | 61 | 32.356 | 23.451 | 37.250 | 1.00 | 4.23 |
| ATOM | 483 | CG | LEU | 61 | 31.400 | 24.019 | 36.212 | 1.00 | 4.22 |
| ATOM | 484 | CD1 | LEU | 61 | 30.116 | 24.482 | 36.841 | 1.00 | 2.46 |
| ATOM | 485 | CD2 | LEU | 61 | 32.030 | 25.178 | 35.413 | 1.00 | 4.72 |
| ATOM | 486 | C | LEU | 61 | 31.694 | 21.018 | 37.026 | 1.00 | 5.60 |
| ATOM | 487 | O | LEU | 61 | 32.750 | 20.517 | 36.638 | 1.00 | 6.42 |
| ATOM | 488 | N | ALA | 62 | 30.504 | 20.552 | 36.686 | 1.00 | 5.63 |
| ATOM | 489 | CA | ALA | 62 | 30.418 | 19.406 | 35.741 | 1.00 | 4.65 |
| ATOM | 490 | CB | ALA | 62 | 29.619 | 18.292 | 36.364 | 1.00 | 2.45 |
| ATOM | 491 | C | ALA | 62 | 29.793 | 19.935 | 34.451 | 1.00 | 5.95 |
| ATOM | 492 | O | ALA | 62 | 28.964 | 20.817 | 34.490 | 1.00 | 6.05 |
| ATOM | 493 | N | ALA | 63 | 30.228 | 19.393 | 33.287 | 1.00 | 6.80 |
| ATOM | 494 | CA | ALA | 63 | 29.749 | 19.839 | 31.985 | 1.00 | 6.73 |
| ATOM | 495 | CB | ALA | 63 | 30.529 | 19.136 | 30.903 | 1.00 | 4.62 |
| ATOM | 496 | C | ALA | 63 | 28.270 | 19.965 | 31.891 | 1.00 | 8.06 |
| ATOM | 497 | O | ALA | 63 | 27.704 | 20.987 | 31.462 | 1.00 | 7.93 |
| ATOM | 498 | N | PRO | 64 | 27.455 | 19.043 | 32.424 | 1.00 | 9.12 |
| ATOM | 499 | CA | PRO | 64 | 25.985 | 19.168 | 32.412 | 1.00 | 8.90 |
| ATOM | 500 | CB | PRO | 64 | 25.605 | 17.981 | 33.293 | 1.00 | 8.17 |
| ATOM | 501 | CG | PRO | 64 | 26.622 | 16.974 | 32.833 | 1.00 | 7.79 |
| ATOM | 502 | CD | PRO | 64 | 27.917 | 17.751 | 32.936 | 1.00 | 8.59 |
| ATOM | 503 | C | PRO | 64 | 25.470 | 20.474 | 32.967 | 1.00 | 10.82 |
| ATOM | 504 | O | PRO | 64 | 24.417 | 21.004 | 32.529 | 1.00 | 12.14 |
| ATOM | 505 | N | GLN | 65 | 26.166 | 21.045 | 33.961 | 1.00 | 9.00 |
| ATOM | 506 | CA | GLN | 65 | 25.887 | 22.265 | 34.602 | 1.00 | 9.54 |
| ATOM | 507 | CB | GLN | 65 | 26.745 | 22.513 | 35.855 | 1.00 | 7.85 |
| ATOM | 508 | CG | GLN | 65 | 26.491 | 21.503 | 36.986 | 1.00 | 7.50 |
| ATOM | 509 | CD | GLN | 65 | 27.298 | 21.965 | 38.217 | 1.00 | 7.64 |
| ATOM | 510 | OE1 | GLN | 65 | 28.430 | 21.506 | 38.343 | 1.00 | 6.16 |
| ATOM | 511 | NE2 | GLN | 65 | 26.694 | 22.855 | 39.036 | 1.00 | 5.03 |
| ATOM | 512 | C | GLN | 65 | 25.965 | 23.523 | 33.712 | 1.00 | 9.56 |
| ATOM | 513 | O | GLN | 65 | 25.400 | 24.560 | 34.092 | 1.00 | 9.71 |
| ATOM | 514 | N | ILE | 66 | 26.607 | 23.468 | 32.580 | 1.00 | 9.17 |
| ATOM | 515 | CA | ILE | 66 | 26.768 | 24.427 | 31.569 | 1.00 | 8.61 |
| ATOM | 516 | CB | ILE | 66 | 28.175 | 24.958 | 31.202 | 1.00 | 7.57 |
| ATOM | 517 | CG1 | ILE | 66 | 29.148 | 23.899 | 30.706 | 1.00 | 7.91 |
| ATOM | 518 | CD1 | ILE | 66 | 30.433 | 24.581 | 30.125 | 1.00 | 7.44 |
| ATOM | 519 | CG2 | ILE | 66 | 28.749 | 25.769 | 32.340 | 1.00 | 7.53 |
| ATOM | 520 | C | ILE | 66 | 26.068 | 23.881 | 30.298 | 1.00 | 9.39 |
| ATOM | 521 | O | ILE | 66 | 26.309 | 24.232 | 29.163 | 1.00 | 10.40 |
| ATOM | 522 | N | ASN | 67 | 25.126 | 22.986 | 30.510 | 1.00 | 10.49 |
| ATOM | 523 | CA | ASN | 67 | 24.266 | 22.369 | 29.557 | 1.00 | 11.57 |
| ATOM | 524 | CB | ASN | 67 | 23.399 | 23.500 | 28.929 | 1.00 | 12.56 |
| ATOM | 525 | CG | ASN | 67 | 22.171 | 22.934 | 28.230 | 1.00 | 12.16 |
| ATOM | 526 | CD1 | ASN | 67 | 21.751 | 21.813 | 28.405 | 1.00 | 12.53 |
| ATOM | 527 | ND2 | ASN | 67 | 21.492 | 23.785 | 27.485 | 1.00 | 14.18 |
| ATOM | 528 | C | ASN | 67 | 24.980 | 21.596 | 28.464 | 1.00 | 11.97 |
| ATOM | 529 | O | ASN | 67 | 24.610 | 21.590 | 27.263 | 1.00 | 12.28 |
| ATOM | 530 | N | ILE | 68 | 26.093 | 21.000 | 28.821 | 1.00 | 10.89 |
| ATOM | 531 | CA | ILE | 68 | 26.918 | 20.161 | 27.888 | 1.00 | 10.88 |
| ATOM | 532 | CB | ILE | 68 | 28.298 | 20.745 | 27.721 | 1.00 | 10.24 |
| ATOM | 533 | CG1 | ILE | 68 | 28.286 | 22.078 | 26.953 | 1.00 | 9.48 |
| ATOM | 534 | CD1 | ILE | 68 | 29.646 | 22.766 | 26.933 | 1.00 | 7.35 |
| ATOM | 535 | CG2 | ILE | 68 | 29.224 | 19.745 | 26.992 | 1.00 | 9.60 |
| ATOM | 536 | C | ILE | 68 | 26.868 | 18.749 | 28.499 | 1.00 | 10.44 |
| ATOM | 537 | O | ILE | 68 | 27.477 | 18.497 | 29.537 | 1.00 | 9.24 |
| ATOM | 538 | N | SER | 69 | 26.091 | 17.859 | 27.888 | 1.00 | 10.07 |
| ATOM | 539 | CA | SER | 69 | 25.867 | 16.541 | 28.468 | 1.00 | 10.16 |
| ATOM | 540 | CB | SER | 69 | 24.475 | 16.071 | 28.036 | 1.00 | 8.68 |
| ATOM | 541 | OG | SER | 69 | 23.982 | 14.974 | 28.781 | 1.00 | 9.95 |
| ATOM | 542 | C | SER | 69 | 26.972 | 15.562 | 28.156 | 1.00 | 10.74 |
| ATOM | 543 | O | SER | 69 | 26.778 | 14.529 | 27.532 | 1.00 | 11.54 |
| ATOM | 544 | N | LYS | 70 | 28.184 | 15.911 | 28.570 | 1.00 | 10.10 |
| ATOM | 545 | CA | LYS | 70 | 29.409 | 15.153 | 28.370 | 1.00 | 9.96 |
| ATOM | 546 | CB | LYS | 70 | 30.368 | 15.890 | 27.428 | 1.00 | 10.10 |
| ATOM | 547 | CG | LYS | 70 | 29.624 | 16.156 | 26.075 | 1.00 | 13.10 |
| ATOM | 548 | CD | LYS | 70 | 30.666 | 16.689 | 25.078 | 1.00 | 15.02 |
| ATOM | 549 | CE | LYS | 70 | 30.002 | 16.829 | 23.705 | 1.00 | 15.43 |
| ATOM | 550 | NZ | LYS | 70 | 30.955 | 17.277 | 22.697 | 1.00 | 17.50 |
| ATOM | 551 | C | LYS | 70 | 30.082 | 14.8R8 | 29.722 | 1.00 | 9.28 |
| ATOM | 552 | O | LYS | 70 | 29.937 | 15.649 | 30.656 | 1.00 | 10.48 |
| ATOM | 553 | N | ARG | 71 | 30.789 | 13.750 | 29.827 | 1.00 | 7.46 |
| ATOM | 554 | CA | ARG | 71 | 31.383 | 13.295 | 31.076 | 1.00 | 7.42 |
| ATOM | 555 | CB | ARG | 71 | 31.426 | 11.762 | 31.151 | 1.00 | 5.24 |
| ATOM | 556 | CG | ARG | 71 | 30.124 | 11.043 | 30.888 | 1.00 | 4.69 |
| ATOM | 557 | CD | ARG | 71 | 30.141 | 9.561 | 31.166 | 1.00 | 3.45 |
| ATOM | 558 | NE | ARG | 71 | 30.097 | 9.086 | 32.533 | 1.00 | 5.53 |
| ATOM | 559 | CZ | ARG | 71 | 29.004 | 9.030 | 33.279 | 1.00 | 6.30 |
| ATOM | 560 | NH1 | ARG | 71 | 27.846 | 9.361 | 32.738 | 1.00 | 6.87 |
| ATOM | 561 | NH2 | ARG | 71 | 28.983 | 8.718 | 34.567 | 1.00 | 9.93 |
| ATOM | 562 | C | ARG | 71 | 32.756 | 13.852 | 31.351 | 1.00 | 7.34 |
| ATOM | 563 | O | ARG | 71 | 33.782 | 13.219 | 31.176 | 1.00 | 7.04 |
| ATOM | 564 | N | MET | 72 | 32.764 | 15.142 | 31.733 | 1.00 | 8.16 |
| ATOM | 565 | CA | MET | 72 | 33.929 | 15.899 | 32.038 | 1.00 | 7.49 |
| ATOM | 566 | CB | MET | 72 | 34.226 | 16.975 | 30.968 | 1.00 | 11.49 |
| ATOM | 567 | CG | MET | 72 | 34.482 | 16.514 | 29.597 | 1.00 | 14.92 |
| ATOM | 568 | SD | MET | 72 | 34.929 | 17.771 | 28.426 | 1.00 | 14.46 |
| ATOM | 569 | CE | MET | 72 | 33.462 | 18.708 | 28.193 | 1.00 | 15.90 |
| ATOM | 570 | C | MET | 72 | 33.639 | 16.756 | 33.276 | 1.00 | 6.05 |
| ATOM | 571 | O | MET | 72 | 32.583 | 17.367 | 33.360 | 1.00 | 5.46 |
| ATOM | 572 | N | ILE | 73 | 34.610 | 16.921 | 34.129 | 1.00 | 5.83 |
| ATOM | 573 | CA | ILE | 73 | 34.470 | 17.767 | 35.294 | 1.00 | 4.01 |
| ATOM | 574 | CB | ILE | 73 | 34.134 | 16.939 | 36.559 | 1.00 | 4.50 |
| ATOM | 575 | CG1 | ILE | 73 | 35.208 | 15.825 | 36.788 | 1.00 | 3.12 |
| ATOM | 576 | CD1 | ILE | 73 | 35.070 | 15.150 | 38.142 | 1.00 | 3.42 |
| ATOM | 577 | CG2 | ILE | 73 | 32.758 | 16.373 | 36.503 | 1.00 | 3.16 |
| ATOM | 578 | C | ILE | 73 | 35.728 | 18.596 | 35.564 | 1.00 | 4.80 |
| ATOM | 579 | O | ILE | 73 | 36.814 | 18.324 | 35.070 | 1.00 | 4.39 |
| ATOM | 580 | N | ALA | 74 | 35.570 | 19.655 | 36.382 | 1.00 | 4.79 |
| ATOM | 581 | CA | ALA | 74 | 36.744 | 20.441 | 36.785 | 1.00 | 5.15 |
| ATOM | 582 | CB | ALA | 74 | 36.868 | 21.730 | 36.054 | 1.00 | 5.71 |
| ATOM | 583 | C | ALA | 74 | 36.659 | 20.559 | 38.308 | 1.00 | 4.79 |
| ATOM | 584 | O | ALA | 74 | 35.592 | 20.723 | 38.844 | 1.00 | 4.28 |
| ATOM | 585 | N | VAL | 75 | 37.774 | 20.324 | 39.020 | 1.00 | 6.37 |
| ATOM | 586 | CA | VAL | 75 | 37.811 | 20.449 | 40.484 | 1.00 | 6.45 |
| ATOM | 587 | CB | VAL | 75 | 37.984 | 19.144 | 41.290 | 1.00 | 5.03 |
| ATOM | 588 | CG1 | VAL | 75 | 38.075 | 19.427 | 42.796 | 1.00 | 2.00 |
| ATOM | 589 | CG2 | VAL | 75 | 36.744 | 18.271 | 41.056 | 1.00 | 3.76 |
| ATOM | 590 | C | VAL | 75 | 38.918 | 21.416 | 40.854 | 1.00 | 7.44 |
| ATOM | 591 | O | VAL | 75 | 40.043 | 21.229 | 40.321 | 1.00 | 8.86 |
| ATOM | 592 | N | LEU | 76 | 38.635 | 22.413 | 41.703 | 1.00 | 6.88 |
| ATOM | 593 | CA | LEU | 76 | 39.681 | 23.368 | 42.027 | 1.00 | 7.77 |
| ATOM | 594 | CB | LEU | 76 | 39.537 | 24.660 | 41.205 | 1.00 | 7.33 |
| ATOM | 595 | CG | LEU | 76 | 40.640 | 25.705 | 41.437 | 1.00 | 4.71 |
| ATOM | 596 | CD1 | LEU | 76 | 41.961 | 25.218 | 40.861 | 1.00 | 3.93 |
| ATOM | 597 | CD2 | LEU | 76 | 40.257 | 27.038 | 40.857 | 1.00 | 7.52 |
| ATOM | 598 | C | LEU | 76 | 39.614 | 23.683 | 43.527 | 1.00 | 8.83 |
| ATOM | 599 | O | LEU | 76 | 38.896 | 24.560 | 43.956 | 1.00 | 10.55 |
| ATOM | 600 | N | ILE | 77 | 40.247 | 22.867 | 44.342 | 1.00 | 8.82 |
| ATOM | 601 | CA | ILE | 77 | 40.215 | 23.024 | 45.796 | 1.00 | 11.03 |
| ATOM | 602 | CB | ILE | 77 | 40.001 | 21.637 | 46.472 | 1.00 | 10.29 |
| ATOM | 603 | CG1 | HIS | 77 | 38.674 | 21.025 | 46.000 | 1.00 | 9.23 |
| ATOM | 604 | CD1 | ILE | 77 | 38.561 | 19.534 | 46.388 | 1.00 | 7.45 |
| ATOM | 605 | CG2 | ILE | 77 | 39.946 | 21.906 | 48.012 | 1.00 | 11.93 |
| ATOM | 606 | C | ILE | 77 | 41.570 | 23.599 | 46.247 | 1.00 | 11.22 |
| ATOM | 607 | O | ILE | 77 | 42.605 | 22.996 | 46.058 | 1.00 | 11.78 |
| ATOM | 608 | N | PRO | 78 | 41.549 | 24.801 | 46.769 | 1.00 | 12.81 |
| ATOM | 609 | CA | PRO | 78 | 42.755 | 25.478 | 47.215 | 1.00 | 13.82 |
| ATOM | 610 | CB | PRO | 78 | 42.281 | 26.881 | 47.476 | 1.00 | 13.82 |
| ATOM | 611 | CG | PRO | 78 | 40.832 | 26.828 | 47.688 | 1.00 | 13.52 |
| ATOM | 612 | CD | PRO | 78 | 40.323 | 25.600 | 47.015 | 1.00 | 13.68 |
| ATOM | 613 | C | PRO | 78 | 43.384 | 24.865 | 48.463 | 1.00 | 14.04 |
| ATOM | 614 | O | PRO | 78 | 42.729 | 24.162 | 49.225 | 1.00 | 13.74 |
| ATOM | 615 | N | ASP | 79 | 44.663 | 25.190 | 48.665 | 1.00 | 15.66 |
| ATOM | 616 | CA | ASP | 79 | 45.421 | 24.733 | 49.796 | 1.00 | 16.97 |
| ATOM | 617 | CB | ASP | 79 | 46.815 | 25.342 | 49.773 | 1.00 | 17.17 |
| ATOM | 618 | CG | ASP | 79 | 47.725 | 24.835 | 50.845 | 1.00 | 19.81 |
| ATOM | 619 | OD1 | ASP | 79 | 47.281 | 24.237 | 51.833 | 1.00 | 21.59 |
| ATOM | 620 | OD2 | ASP | 79 | 48.968 | 24.997 | 50.743 | 1.00 | 23.71 |
| ATOM | 621 | C | ASP | 79 | 44.730 | 25.095 | 51.103 | 1.00 | 19.47 |
| ATOM | 622 | O | ASP | 79 | 44.386 | 26.257 | 51.328 | 1.00 | 19.81 |
| ATOM | 623 | N | ASP | 80 | 44.523 | 24.114 | 51.958 | 1.00 | 21.56 |
| ATOM | 624 | CA | ASP | 80 | 43.899 | 24.283 | 53.240 | 1.00 | 24.85 |
| ATOM | 625 | CB | ASP | 80 | 43.294 | 22.944 | 53.706 | 1.00 | 27.72 |
| ATOM | 626 | CG | ASP | 80 | 44.345 | 21.869 | 53.833 | 1.00 | 30.53 |
| ATOM | 627 | OD1 | ASP | 80 | 44.094 | 20.790 | 54.401 | 1.00 | 32.70 |
| ATOM | 628 | OD2 | ASP | 80 | 45.490 | 22.015 | 53.347 | 1.00 | 30.54 |
| ATOM | 629 | C | ASP | 80 | 44.898 | 24.740 | 54.318 | 1.00 | 26.28 |
| ATOM | 630 | O | ASP | 80 | 44.491 | 25.228 | 55.374 | 1.00 | 27.81 |
| ATOM | 631 | N | GLY | 81 | 46.168 | 24.602 | 54.112 | 1.00 | 26.90 |
| ATOM | 632 | CA | GLY | 81 | 47.214 | 24.943 | 55.079 | 1.00 | 27.17 |
| ATOM | 633 | C | GLY | 81 | 48.062 | 23.658 | 55.240 | 1.00 | 27.82 |
| ATOM | 634 | O | GLY | 81 | 49.259 | 23.728 | 55.546 | 1.00 | 28.21 |
| ATOM | 635 | N | SER | 82 | 47.391 | 22.520 | 54.989 | 1.00 | 27.02 |
| ATOM | 636 | CA | SER | 82 | 48.108 | 21.246 | 55.049 | 1.00 | 26.04 |
| ATOM | 637 | CB | SER | 82 | 47.248 | 20.019 | 54.796 | 1.00 | 25.15 |
| ATOM | 638 | OG | SER | 82 | 46.765 | 20.059 | 53.446 | 1.00 | 26.13 |
| ATOM | 639 | C | SER | 82 | 49.127 | 21.195 | 53.882 | 1.00 | 24.89 |
| ATOM | 640 | O | SER | 82 | 50.066 | 20.426 | 53.980 | 1.00 | 25.62 |
| ATOM | 641 | N | GLY | 83 | 48.917 | 22.012 | 52.872 | 1.00 | 23.48 |
| ATOM | 642 | CA | GLY | 83 | 49.841 | 21.974 | 51.719 | 1.00 | 21.77 |
| ATOM | 643 | C | GLY | 83 | 49.163 | 21.092 | 50.638 | 1.00 | 19.26 |
| ATOM | 644 | O | GLY | 83 | 49.801 | 20.902 | 49.631 | 1.00 | 20.21 |
| ATOM | 645 | N | LYS | 84 | 47.972 | 20.576 | 50.867 | 1.00 | 16.73 |
| ATOM | 646 | CA | LYS | 84 | 47.296 | 19.797 | 49.836 | 1.00 | 14.79 |
| ATOM | 647 | CB | LYS | 84 | 46.528 | 18.644 | 50.415 | 1.00 | 16.65 |
| ATOM | 648 | CG | LYS | 84 | 47.294 | 17.703 | 51.325 | 1.00 | 20.53 |
| ATOM | 649 | CD | LYS | 84 | 46.320 | 16.536 | 51.571 | 1.00 | 26.25 |
| ATOM | 650 | CE | LYS | 84 | 46.765 | 15.630 | 52.681 | 1.00 | 29.35 |
| ATOM | 651 | NZ | LYS | 84 | 46.698 | 16.382 | 53.997 | 1.00 | 31.51 |
| ATOM | 652 | C | LYS | 84 | 46.305 | 20.645 | 49.058 | 1.00 | 13.36 |
| ATOM | 653 | O | LYS | 84 | 45.482 | 21.354 | 49.657 | 1.00 | 13.57 |
| ATOM | 654 | N | SER | 85 | 46.334 | 20.575 | 47.727 | 1.00 | 12.20 |
| ATOM | 655 | CA | SER | 85 | 45.399 | 21.352 | 46.904 | 1.00 | 10.79 |
| ATOM | 656 | CB | SER | 85 | 45.910 | 22.713 | 46.473 | 1.00 | 10.03 |
| ATOM | 657 | CG | SER | 85 | 46.979 | 22.545 | 45.587 | 1.00 | 12.27 |
| ATOM | 658 | C | SER | 85 | 45.052 | 20.517 | 45.660 | 1.00 | 9.97 |
| ATOM | 659 | O | SER | 85 | 45.892 | 19.710 | 45.314 | 1.00 | 9.45 |
| ATOM | 660 | N | TYR | 86 | 43.855 | 20.667 | 45.136 | 1.00 | 8.93 |
| ATOM | 661 | CA | TYR | 86 | 43.370 | 19.823 | 44.040 | 1.00 | 8.91 |
| ATOM | 662 | CB | TYR | 86 | 42.190 | 18.985 | 44.568 | 1.00 | 9.61 |
| ATOM | 663 | CG | TYR | 86 | 42.612 | 18.127 | 45.766 | 1.00 | 11.87 |
| ATOM | 664 | CD1 | TYR | 86 | 42.525 | 18.675 | 47.031 | 1.00 | 12.22 |
| ATOM | 665 | CE1 | TYR | 86 | 42.968 | 17.980 | 48.146 | 1.00 | 14.09 |
| ATOM | 666 | CZ | TYR | 86 | 43.464 | 16.693 | 47.998 | 1.00 | 13.74 |
| ATOM | 667 | OH | TYR | 86 | 43.877 | 16.050 | 49.147 | 1.00 | 14.04 |
| ATOM | 668 | CE2 | TYR | 86 | 43.587 | 16.139 | 46.747 | 1.00 | 13.17 |
| ATOM | 669 | CD2 | TYR | 86 | 43.136 | 16.849 | 45.631 | 1.00 | 12.81 |
| ATOM | 670 | C | TYR | 86 | 42.937 | 20.636 | 42.843 | 1.00 | 8.39 |
| ATOM | 671 | O | TYR | 86 | 42.041 | 21.470 | 42.905 | 1.00 | 11.10 |
| ATOM | 672 | N | ASP | 87 | 43.597 | 20.487 | 41.740 | 1.00 | 7.58 |
| ATOM | 673 | CA | ASP | 87 | 43.388 | 21.198 | 40.491 | 1.00 | 7.93 |
| ATOM | 674 | CB | ASP | 87 | 44.561 | 22.172 | 40.249 | 1.00 | 7.54 |
| ATOM | 675 | CG | ASP | 87 | 44.336 | 23.088 | 39.060 | 1.00 | 9.36 |
| ATOM | 676 | OD1 | ASP | 87 | 43.367 | 22.912 | 38.300 | 1.00 | 9.22 |
| ATOM | 677 | OD2 | ASP | 87 | 45.098 | 24.043 | 38.908 | 1.00 | 12.88 |
| ATOM | 678 | C | ASP | 87 | 43.369 | 20.124 | 39.392 | 1.00 | 6.63 |
| ATOM | 679 | O | ASP | 87 | 44.445 | 19.695 | 38.965 | 1.00 | 7.41 |
| ATOM | 680 | N | TYR | 88 | 42.204 | 19.657 | 39.046 | 1.00 | 7.18 |
| ATOM | 681 | CA | TYR | 88 | 42.058 | 18.612 | 38.048 | 1.00 | 7.88 |
| ATOM | 682 | CB | TYR | 88 | 41.775 | 17.252 | 38.727 | 1.00 | 8.03 |
| ATOM | 683 | CG | TYR | 88 | 42.918 | 16.706 | 39.528 | 1.00 | 7.31 |
| ATOM | 684 | CD1 | TYR | 88 | 43.051 | 16.986 | 40.887 | 1.00 | 7.10 |
| ATOM | 685 | CE1 | TYR | 88 | 44.093 | 16.455 | 41.622 | 1.00 | 5.16 |
| ATOM | 686 | CZ | TYR | 88 | 45.017 | 15.649 | 40.992 | 1.00 | 5.90 |
| ATOM | 687 | OH | TYR | 88 | 46.088 | 15.153 | 41.721 | 1.00 | 7.39 |
| ATOM | 688 | CE2 | TYR | 88 | 44.955 | 15.404 | 39.643 | 1.00 | 6.03 |
| ATOM | 689 | CD2 | TYR | 88 | 43.866 | 15.894 | 38.917 | 1.00 | 6.97 |
| ATOM | 690 | C | TYR | 88 | 40.939 | 18.810 | 37.051 | 1.00 | 8.22 |
| ATOM | 691 | O | TYR | 88 | 39.816 | 19.193 | 37.355 | 1.00 | 9.63 |
| ATOM | 692 | N | MET | 89 | 41.231 | 18.498 | 35.814 | 1.00 | 8.89 |
| ATOM | 693 | CA | MET | 89 | 40.233 | 18.533 | 34.736 | 1.00 | 8.17 |
| ATOM | 694 | CB | MET | 89 | 40.600 | 19.441 | 33.591 | 1.00 | 7.98 |
| ATOM | 695 | CG | MET | 89 | 40.522 | 20.918 | 33.852 | 1.00 | 8.05 |
| ATOM | 696 | SD | MET | 89 | 41.005 | 21.973 | 32.496 | 1.00 | 8.41 |
| ATOM | 697 | CE | MET | 89 | 42.746 | 21.671 | 32.340 | 1.00 | 7.64 |
| ATOM | 698 | C | MET | 89 | 40.203 | 17.038 | 34.319 | 1.00 | 5.98 |
| ATOM | 699 | O | MET | 89 | 41.212 | 16.525 | 33.797 | 1.00 | 6.63 |
| ATOM | 700 | N | LEU | 90 | 39.123 | 16.386 | 34.611 | 1.00 | 4.43 |
| ATOM | 701 | CA | LEU | 90 | 39.050 | 14.952 | 34.284 | 1.00 | 5.33 |
| ATOM | 702 | CB | LEU | 90 | 38.707 | 14.179 | 35.541 | 1.00 | 6.25 |
| ATOM | 703 | CG | LEU | 90 | 39.564 | 14.222 | 36.755 | 1.00 | 6.92 |
| ATOM | 704 | CD1 | LEU | 90 | 39.034 | 13.233 | 37.827 | 1.00 | 4.71 |
| ATOM | 705 | CD2 | LEU | 90 | 41.018 | 13.889 | 36.453 | 1.00 | 6.93 |
| ATOM | 706 | C | LEU | 90 | 38.027 | 14.648 | 33.196 | 1.00 | 4.75 |
| ATOM | 707 | O | LEU | 90 | 36.963 | 15.237 | 33.151 | 1.00 | 5.61 |
| ATOM | 708 | N | VAL | 91 | 38.354 | 13.719 | 32.348 | 1.00 | 4.98 |
| ATOM | 709 | CA | VAL | 91 | 37.487 | 13.246 | 31.253 | 1.00 | 4.58 |
| ATOM | 710 | CB | VAL | 91 | 38.323 | 13.299 | 29.938 | 1.00 | 4.51 |
| ATOM | 711 | CG1 | VAL | 91 | 37.590 | 12.571 | 28.815 | 1.00 | 6.97 |
| ATOM | 712 | CG2 | VAL | 91 | 38.589 | 14.718 | 29.522 | 1.00 | 2.32 |
| ATOM | 713 | C | VAL | 91 | 37.137 | 11.805 | 31.576 | 1.00 | 5.04 |
| ATOM | 714 | O | VAL | 91 | 38.003 | 11.030 | 31.991 | 1.00 | 5.81 |
| ATOM | 715 | N | ASN | 92 | 35.842 | 11.420 | 31.481 | 1.00 | 5.85 |
| ATOM | 716 | CA | ASN | 92 | 35.357 | 10.088 | 31.747 | 1.00 | 6.19 |
| ATOM | 717 | CB | ASN | 92 | 35.743 | 9.111 | 30.644 | 1.00 | 7.29 |
| ATOM | 718 | CG | ASN | 92 | 35.308 | 9.457 | 29.252 | 1.00 | 7.92 |
| ATOM | 719 | OD1 | ASN | 92 | 34.247 | 10.045 | 29.090 | 1.00 | 7.73 |
| ATOM | 720 | ND2 | ASN | 92 | 36.092 | 9.017 | 28.271 | 1.00 | 10.17 |
| ATOM | 721 | C | ASN | 92 | 35.756 | 9.501 | 33.083 | 1.00 | 8.19 |
| ATOM | 722 | O | ASN | 92 | 36.265 | 8.363 | 33.177 | 1.00 | 9.98 |
| ATOM | 723 | N | PRO | 93 | 35.644 | 10.283 | 34.153 | 1.00 | 8.97 |
| ATOM | 724 | CD | PRO | 93 | 35.059 | 11.684 | 34.132 | 1.00 | 7.81 |
| ATOM | 725 | CA | PRO | 93 | 35.996 | 9.859 | 35.482 | 1.00 | 8.04 |
| ATOM | 726 | CB | PRO | 93 | 35.928 | 11.155 | 36.272 | 1.00 | 7.31 |
| ATOM | 727 | CG | PRO | 93 | 34.802 | 11.890 | 35.596 | 1.00 | 7.53 |
| ATOM | 728 | C | PRO | 93 | 35.074 | 8.770 | 35.997 | 1.00 | 9.49 |
| ATOM | 729 | O | PRO | 93 | 33.852 | 8.750 | 35.756 | 1.00 | 9.28 |
| ATOM | 730 | N | LYS | 94 | 35.690 | 7.828 | 36.740 | 1.00 | 9.93 |
| ATOM | 731 | CA | LYS | 94 | 34.921 | 6.724 | 37.302 | 1.00 | 9.81 |
| ATOM | 732 | CB | LYS | 94 | 34.975 | 5.537 | 36.280 | 1.00 | 13.20 |
| ATOM | 733 | CG | LYS | 94 | 33.920 | 4.483 | 36.579 | 1.00 | 18.63 |
| ATOM | 734 | CD | LYS | 94 | 34.020 | 3.251 | 35.668 | 1.00 | 23.45 |
| ATOM | 735 | CE | LYS | 94 | 32.846 | 2.297 | 35.911 | 1.00 | 25.89 |
| ATOM | 736 | NZ | LYS | 94 | 32.998 | 1.075 | 35.012 | 1.00 | 28.49 |
| ATOM | 737 | C | LYS | 94 | 35.539 | 6.242 | 38.610 | 1.00 | 8.51 |
| ATOM | 738 | O | LYS | 94 | 36.749 | 6.036 | 38.748 | 1.00 | 6.81 |
| ATOM | 739 | N | ILE | 95 | 34.686 | 6.078 | 39.604 | 1.00 | 7.95 |
| ATOM | 740 | CA | ILE | 95 | 35.040 | 5.534 | 40.910 | 1.00 | 6.10 |
| ATOM | 741 | CB | ILE | 95 | 34.014 | 5.898 | 41.954 | 1.00 | 5.18 |
| ATOM | 742 | CG1 | ILE | 95 | 34.046 | 7.444 | 42.211 | 1.00 | 6.06 |
| ATOM | 743 | CD1 | ILE | 95 | 32.837 | 7.904 | 43.045 | 1.00 | 5.94 |
| ATOM | 744 | CG2 | ILE | 95 | 34.329 | 5.157 | 43.281 | 1.00 | 5.58 |
| ATOM | 745 | C | ILE | 95 | 35.166 | 3.997 | 40.716 | 1.00 | 6.29 |
| ATOM | 746 | O | ILE | 95 | 34.198 | 3.309 | 40.347 | 1.00 | 5.34 |
| ATOM | 747 | N | VAL | 96 | 36.391 | 3.492 | 40.859 | 1.00 | 4.94 |
| ATOM | 748 | CA | VAL | 96 | 36.632 | 2.073 | 40.636 | 1.00 | 5.84 |
| ATOM | 749 | CB | VAL | 96 | 37.831 | 1.745 | 39.776 | 1.00 | 5.80 |
| ATOM | 750 | CG1 | VAL | 96 | 37.679 | 2.310 | 38.351 | 1.00 | 6.24 |
| ATOM | 751 | CG2 | VAL | 96 | 39.165 | 2.258 | 40.318 | 1.00 | 5.22 |
| ATOM | 752 | C | VAL | 96 | 36.608 | 1.295 | 41.950 | 1.00 | 5.83 |
| ATOM | 753 | O | VAL | 96 | 36.279 | 0.126 | 41.914 | 1.00 | 5.47 |
| ATOM | 754 | N | SER | 97 | 36.847 | 1.959 | 43.077 | 1.00 | 5.14 |
| ATOM | 755 | CA | EER | 97 | 36.830 | 1.376 | 44.383 | 1.00 | 5.56 |
| ATOM | 756 | CB | SER | 97 | 38.128 | 0.683 | 44.766 | 1.00 | 3.06 |
| ATOM | 757 | OG | SER | 97 | 39.236 | 1.451 | 44.594 | 1.00 | 6.94 |
| ATOM | 758 | C | SER | 97 | 36.592 | 2.485 | 45.447 | 1.00 | 6.21 |
| ATOM | 759 | O | SER | 97 | 36.842 | 3.650 | 45.154 | 1.00 | 5.16 |
| ATOM | 760 | N | HIS | 98 | 36.080 | 2.045 | 46.577 | 1.00 | 5.51 |
| ATOM | 761 | CA | HIS | 98 | 35.807 | 2.912 | 47.695 | 1.00 | 6.19 |
| ATOM | 762 | CB | HIS | 98 | 34.521 | 3.705 | 47.410 | 1.00 | 5.68 |
| ATOM | 763 | CG | HIS | 98 | 33.314 | 2.839 | 47.189 | 1.00 | 3.10 |
| ATOM | 764 | ND2 | HIS | 98 | 32.540 | 2.415 | 48.249 | 1.00 | 5.80 |
| ATOM | 765 | CE1 | HIS | 98 | 31.533 | 1.690 | 47.820 | 1.00 | 3.91 |
| ATOM | 766 | NE2 | HIS | 98 | 31.630 | 1.631 | 46.504 | 1.00 | 4.11 |
| ATOM | 767 | CD2 | HIS | 98 | 32.731 | 2.333 | 46.100 | 1.00 | 2.55 |
| ATOM | 768 | C | HIS | 98 | 35.718 | 2.187 | 49.043 | 1.00 | 6.46 |
| ATOM | 769 | O | HIS | 98 | 35.484 | 0.998 | 49.174 | 1.00 | 5.57 |
| ATOM | 770 | N | SER | 99 | 35.882 | 2.968 | 50.123 | 1.00 | 6.76 |
| ATOM | 771 | CA | SER | 99 | 35.744 | 2.476 | 51.476 | 1.00 | 6.43 |
| ATOM | 772 | CB | SER | 99 | 36.188 | 3.588 | 52.459 | 1.00 | 7.38 |
| ATOM | 773 | OG | SER | 99 | 35.341 | 4.757 | 52.354 | 1.00 | 7.60 |
| ATOM | 774 | C | SER | 99 | 34.290 | 2.177 | 51.765 | 1.00 | 6.67 |
| ATOM | 775 | O | SER | 99 | 33.365 | 2.734 | 51.153 | 1.00 | 7.55 |
| ATOM | 776 | N | VAL | 100 | 34.005 | 1.310 | 52.714 | 1.00 | 6.58 |
| ATOM | 777 | CA | VAL | 100 | 32.615 | 1.043 | 53.160 | 1.00 | 6.28 |
| ATOM | 778 | CB | VAL | 100 | 32.598 | −0.278 | 53.971 | 1.00 | 6.35 |
| ATOM | 779 | CG1 | VAL | 100 | 31.215 | −0.420 | 54.605 | 1.00 | 6.97 |
| ATOM | 780 | CG2 | VAL | 100 | 32.745 | −1.461 | 52.932 | 1.00 | 6.04 |
| ATOM | 781 | C | VAL | 100 | 32.175 | 2.219 | 54.062 | 1.00 | 6.51 |
| ATOM | 782 | O | VAL | 100 | 31.049 | 2.745 | 54.044 | 1.00 | 6.77 |
| ATOM | 783 | N | GLN | 101 | 33.124 | 2.741 | 54.816 | 1.00 | 7.40 |
| ATOM | 784 | CA | GLN | 101 | 32.865 | 3.871 | 55.734 | 1.00 | 9.68 |
| ATOM | 785 | CB | GLN | 101 | 34.012 | 4.032 | 56.704 | 1.00 | 9.29 |
| ATOM | 786 | CG | GLN | 101 | 33.909 | 5.192 | 57.649 | 1.00 | 9.97 |
| ATOM | 787 | CD | GLN | 101 | 35.017 | 5.329 | 58.642 | 1.00 | 10.63 |
| ATOM | 788 | OE1 | GLN | 101 | 36.190 | 5.548 | 58.257 | 1.00 | 13.55 |
| ATOM | 789 | NE2 | GLN | 101 | 34.656 | 5.267 | 59.887 | 1.00 | 11.28 |
| ATOM | 790 | C | GLN | 101 | 32.569 | 5.189 | 54.993 | 1.00 | 9.37 |
| ATOM | 791 | O | GLN | 101 | 33.236 | 5.551 | 54.068 | 1.00 | 7.54 |
| ATOM | 792 | N | GLU | 102 | 31.480 | 5.845 | 55.472 | 1.00 | 9.24 |
| ATOM | 793 | CA | GLU | 102 | 31.008 | 7.074 | 54.921 | 1.00 | 9.90 |
| ATOM | 794 | CB | GLU | 102 | 29.464 | 7.141 | 55.018 | 1.00 | 11.63 |
| ATOM | 795 | CG | GLU | 102 | 28.763 | 6.013 | 54.261 | 1.00 | 12.22 |
| ATOM | 796 | CD | GLU | 102 | 27.278 | 6.126 | 54.402 | 1.00 | 14.95 |
| ATOM | 797 | OE1 | GLU | 102 | 26.805 | 6.596 | 55.462 | 1.00 | 18.92 |
| ATOM | 798 | OE2 | GLU | 102 | 26.511 | 5.770 | 53.500 | 1.00 | 16.02 |
| ATOM | 799 | C | GLU | 102 | 31.533 | 8.340 | 55.593 | 1.00 | 10.50 |
| ATOM | 800 | O | GLU | 102 | 32.169 | 8.275 | 56.630 | 1.00 | 11.22 |
| ATOM | 801 | N | ALA | 103 | 31.273 | 9.485 | 54.974 | 1.00 | 9.97 |
| ATOM | 802 | CA | ALA | 103 | 31.699 | 10.772 | 55.541 | 1.00 | 9.74 |
| ATOM | 803 | CB | ALA | 103 | 33.049 | 11.245 | 55.034 | 1.00 | 8.10 |
| ATOM | 804 | C | ALA | 103 | 30.644 | 11.816 | 55.123 | 1.00 | 9.71 |
| ATOM | 805 | O | ALA | 103 | 30.011 | 11.529 | 54.129 | 1.00 | 9.34 |
| ATOM | 806 | N | TYR | 104 | 30.556 | 12.936 | 55.808 | 1.00 | 9.87 |
| ATOM | 807 | CA | TYR | 104 | 29.585 | 13.984 | 55.438 | 1.00 | 11.04 |
| ATOM | 808 | CB | TYR | 104 | 28.192 | 13.707 | 56.083 | 1.00 | 11.01 |
| ATOM | 809 | CG | TYR | 104 | 28.273 | 13.737 | 57.596 | 1.00 | 12.87 |
| ATOM | 810 | CD1 | TYR | 104 | 27.958 | 14.884 | 58.330 | 1.00 | 14.01 |
| ATOM | 811 | CE1 | TYR | 104 | 28.088 | 14.909 | 59.727 | 1.00 | 13.96 |
| ATOM | 812 | CZ | TYR | 104 | 28.519 | 13.774 | 60.389 | 1.00 | 14.95 |
| ATOM | 813 | OH | TYR | 104 | 28.676 | 13.775 | 61.757 | 1.00 | 15.04 |
| ATOM | 814 | CE2 | TYR | 104 | 28.796 | 12.619 | 59.685 | 1.00 | 13.61 |
| ATOM | 815 | CD2 | TYR | 104 | 28.665 | 12.589 | 58.310 | 1.00 | 13.25 |
| ATOM | 816 | C | TYR | 104 | 30.135 | 15.342 | 55.885 | 1.00 | 10.66 |
| ATOM | 817 | O | TYR | 104 | 30.942 | 15.394 | 56.853 | 1.00 | 11.97 |
| ATOM | 818 | N | LEU | 105 | 29.819 | 16.416 | 55.227 | 1.00 | 10.31 |
| ATOM | 819 | CA | LEU | 105 | 30.271 | 17.775 | 55.675 | 1.00 | 10.31 |
| ATOM | 820 | CB | LEU | 105 | 30.363 | 18.745 | 54.499 | 1.00 | 8.28 |
| ATOM | 821 | CG | LEU | 105 | 31.202 | 18.355 | 53.295 | 1.00 | 7.12 |
| ATOM | 822 | CD1 | LEU | 105 | 31.189 | 19.393 | 52.190 | 1.00 | 5.52 |
| ATOM | 823 | CD2 | LEU | 105 | 32.669 | 18.076 | 53.663 | 1.00 | 5.12 |
| ATOM | 824 | C | LEU | 105 | 29.156 | 18.174 | 56.667 | 1.00 | 10.48 |
| ATOM | 825 | O | LEU | 105 | 27.977 | 17.998 | 56.358 | 1.00 | 10.24 |
| ATOM | 826 | N | PRO | 106 | 29.469 | 18.627 | 57.865 | 1.00 | 11.95 |
| ATOM | 827 | CA | PRO | 106 | 28.454 | 18.963 | 58.863 | 1.00 | 12.84 |
| ATOM | 828 | CB | PRO | 106 | 29.276 | 19.275 | 60.093 | 1.00 | 13.00 |
| ATOM | 829 | CG | PRO | 106 | 30.549 | 19.799 | 59.534 | 1.00 | 14.02 |
| ATOM | 830 | CD | PRO | 106 | 30.826 | 18.911 | 58.327 | 1.00 | 13.21 |
| ATOM | 831 | C | PRO | 106 | 27.479 | 20.038 | 58.465 | 1.00 | 13.69 |
| ATOM | 832 | O | PRO | 106 | 26.376 | 20.106 | 59.000 | 1.00 | 14.91 |
| ATOM | 833 | N | THR | 107 | 27.838 | 20.930 | 57.523 | 1.00 | 14.66 |
| ATOM | 834 | CA | THR | 107 | 26.983 | 21.976 | 57.008 | 1.00 | 16.19 |
| ATOM | 835 | CB | THR | 107 | 27.799 | 23.235 | 56.607 | 1.00 | 21.67 |
| ATOM | 836 | OG1 | THR | 107 | 28.885 | 22.852 | 55.716 | 1.00 | 25.40 |
| ATOM | 837 | CG2 | THR | 107 | 28.349 | 23.915 | 57.844 | 1.00 | 24.07 |
| ATOM | 838 | C | THR | 107 | 26.199 | 21.591 | 55.752 | 1.00 | 13.84 |
| ATOM | 839 | O | THR | 107 | 25.549 | 22.430 | 55.155 | 1.00 | 14.95 |
| ATOM | 840 | N | GLY | 108 | 26.232 | 20.326 | 55.368 | 1.00 | 11.38 |
| ATOM | 841 | CA | GLY | 108 | 25.547 | 19.869 | 54.176 | 1.00 | 8.51 |
| ATOM | 842 | C | GLY | 108 | 26.402 | 20.337 | 53.000 | 1.00 | 7.43 |
| ATOM | 843 | O | GLY | 108 | 27.611 | 20.629 | 53.127 | 1.00 | 7.41 |
| ATOM | 844 | N | GLU | 109 | 25.808 | 20.388 | 51.837 | 1.00 | 6.61 |
| ATOM | 845 | CA | GLU | 109 | 26.466 | 20.806 | 50.634 | 1.00 | 7.52 |
| ATOM | 846 | CB | GLU | 109 | 26.686 | 19.615 | 49.685 | 1.00 | 7.66 |
| ATOM | 847 | CG | GLU | 109 | 27.582 | 18.521 | 50.288 | 1.00 | 5.52 |
| ATOM | 848 | CD | GLU | 109 | 27.731 | 17.381 | 49.320 | 1.00 | 7.26 |
| ATOM | 849 | OE1 | GLU | 109 | 27.368 | 17.478 | 48.139 | 1.00 | 7.28 |
| ATOM | 850 | OE2 | GLU | 109 | 28.195 | 16.298 | 49.739 | 1.00 | 8.58 |
| ATOM | 851 | C | GLU | 109 | 25.653 | 21.884 | 49.896 | 1.00 | 8.48 |
| ATOM | 852 | O | GLU | 109 | 24.554 | 22.191 | 50.241 | 1.00 | 10.22 |
| ATOM | 853 | N | GLY | 110 | 26.303 | 22.414 | 48.883 | 1.00 | 10.01 |
| ATOM | 854 | CA | GLY | 110 | 25.702 | 23.424 | 47.989 | 1.00 | 10.84 |
| ATOM | 855 | C | GLY | 110 | 26.178 | 23.098 | 46.548 | 1.00 | 11.21 |
| ATOM | 856 | O | GLY | 110 | 27.056 | 22.281 | 46.342 | 1.00 | 11.00 |
| ATOM | 857 | N | CYS | 111 | 25.608 | 23.776 | 45.582 | 1.00 | 11.21 |
| ATOM | 858 | CA | CYS | 111 | 25.870 | 23.582 | 44.163 | 1.00 | 10.09 |
| ATOM | 859 | C | CYS | 111 | 25.533 | 24.848 | 43.352 | 1.00 | 10.36 |
| ATOM | 860 | O | CYS | 111 | 24.562 | 25.536 | 43.627 | 1.00 | 10.29 |
| ATOM | 861 | CB | CYS | 111 | 24.923 | 22.434 | 43.721 | 1.00 | 9.84 |
| ATOM | 862 | SG | CYS | 111 | 25.048 | 21.924 | 42.045 | 1.00 | 8.95 |
| ATOM | 863 | N | LEU | 112 | 26.398 | 25.177 | 42.403 | 1.00 | 11.20 |
| ATOM | 864 | CA | LEU | 112 | 26.170 | 26.384 | 41.550 | 1.00 | 11.83 |
| ATOM | 865 | CB | LEU | 112 | 27.411 | 26.486 | 40.666 | 1.00 | 12.57 |
| ATOM | 866 | CG | LEU | 112 | 28.708 | 26.878 | 41.352 | 1.00 | 15.06 |
| ATOM | 867 | CD1 | LEU | 112 | 29.871 | 26.352 | 40.469 | 1.00 | 15.77 |
| ATOM | 868 | CD2 | LEU | 112 | 28.926 | 28.394 | 41.340 | 1.00 | 15.61 |
| ATOM | 869 | C | LEU | 112 | 24.851 | 26.350 | 40.836 | 1.00 | 11.60 |
| ATOM | 870 | O | LEU | 112 | 24.228 | 27.383 | 40.488 | 1.00 | 11.36 |
| ATOM | 871 | N | SER | 113 | 24.310 | 25.146 | 40.587 | 1.00 | 11.85 |
| ATOM | 872 | CA | SER | 113 | 23.048 | 24.9S0 | 39.917 | 1.00 | 11.15 |
| ATOM | 873 | CB | SER | 113 | 23.025 | 23.699 | 39.049 | 1.00 | 10.66 |
| ATOM | 874 | OG | SER | 113 | 23.968 | 23.793 | 37.992 | 1.00 | 7.38 |
| ATOM | 875 | C | SER | 113 | 21.886 | 24.945 | 40.885 | 1.00 | 12.54 |
| ATOM | 876 | O | SER | 113 | 20.731 | 24.917 | 40.427 | 1.00 | 14.05 |
| ATOM | 877 | N | VAL | 114 | 22.143 | 24.975 | 42.198 | 1.00 | 11.90 |
| ATOM | 878 | CA | VAL | 114 | 21.022 | 25.016 | 43.143 | 1.00 | 11.54 |
| ATOM | 879 | CB | VAL | 114 | 21.067 | 23.872 | 44.160 | 1.00 | 9.05 |
| ATOM | 880 | CG1 | VAL | 114 | 19.777 | 23.919 | 45.007 | 1.00 | 10.95 |
| ATOM | 881 | CG2 | VAL | 114 | 21.080 | 22.538 | 43.409 | 1.00 | 9.28 |
| ATOM | 882 | C | VAL | 114 | 21.005 | 26.402 | 43.818 | 1.00 | 11.42 |
| ATOM | 883 | O | VAL | 114 | 21.854 | 26.663 | 44.630 | 1.00 | 11.25 |
| ATOM | 884 | N | ASP | 115 | 20.077 | 27.277 | 43.466 | 1.00 | 12.74 |
| ATOM | 885 | CA | ASP | 115 | 20.012 | 28.618 | 43.985 | 1.00 | 15.39 |
| ATOM | 886 | CB | ASP | 115 | 19.029 | 29.540 | 43.310 | 1.00 | 17.73 |
| ATOM | 887 | CG | ASP | 115 | 19.300 | 29.933 | 41.893 | 1.00 | 20.05 |
| ATOM | 888 | OD1 | ASP | 115 | 20.331 | 29.598 | 41.312 | 1.00 | 18.80 |
| ATOM | 889 | OD2 | ASP | 115 | 18.380 | 30.637 | 41.372 | 1.00 | 22.85 |
| ATOM | 890 | C | ASP | 115 | 19.789 | 28.760 | 45.492 | 1.00 | 16.27 |
| ATOM | 891 | O | ASP | 115 | 20.405 | 29.626 | 46.099 | 1.00 | 17.11 |
| ATOM | 892 | N | ASP | 116 | 18.857 | 28.001 | 46.028 | 1.00 | 17.25 |
| ATOM | 893 | CA | ASP | 116 | 18.590 | 28.081 | 47.461 | 1.00 | 18.90 |
| ATOM | 894 | CB | ASP | 116 | 17.117 | 27.717 | 47.739 | 1.00 | 21.30 |
| ATOM | 895 | CG | ASP | 116 | 16.153 | 28.650 | 47.041 | 1.00 | 24.14 |
| ATOM | 896 | OD1 | ASP | 116 | 16.391 | 29.883 | 47.008 | 1.00 | 23.07 |
| ATOM | 897 | OD2 | ASP | 116 | 15.132 | 28.134 | 46.495 | 1.00 | 26.77 |
| ATOM | 898 | C | ASP | 116 | 19.444 | 27.106 | 48.249 | 1.00 | 19.20 |
| ATOM | 899 | O | ASP | 116 | 19.738 | 26.025 | 47.744 | 1.00 | 19.78 |
| ATOM | 900 | N | ASN | 117 | 19.869 | 27.534 | 49.428 | 1.00 | 18.50 |
| ATOM | 901 | CA | ASN | 117 | 20.571 | 26.691 | 50.352 | 1.00 | 18.69 |
| ATOM | 902 | CB | ASN | 117 | 21.143 | 27.363 | 51.585 | 1.00 | 19.43 |
| ATOM | 903 | CG | ASN | 117 | 22.487 | 28.000 | 51.304 | 1.00 | 22.50 |
| ATOM | 904 | OD1 | ASN | 117 | 23.330 | 27.263 | 50.736 | 1.00 | 23.16 |
| ATOM | 905 | ND2 | ASN | 117 | 22.749 | 29.225 | 51.663 | 1.00 | 22.67 |
| ATOM | 906 | C | ASN | 117 | 19.655 | 25.543 | 50.781 | 1.00 | 19.22 |
| ATOM | 907 | O | ASN | 117 | 18.465 | 25.737 | 51.042 | 1.00 | 20.65 |
| ATOM | 908 | N | VAL | 118 | 20.216 | 24.324 | 50.775 | 1.00 | 18.92 |
| ATOM | 909 | CA | VAL | 118 | 19.445 | 23.151 | 51.178 | 1.00 | 18.04 |
| ATOM | 910 | CB | VAL | 118 | 19.364 | 22.090 | 50.077 | 1.00 | 18.23 |
| ATOM | 911 | CG1 | VAL | 118 | 18.474 | 20.906 | 50.480 | 1.00 | 15.62 |
| ATOM | 912 | CG2 | VAL | 118 | 18.797 | 22.712 | 48.813 | 1.00 | 18.51 |
| ATOM | 913 | C | VAL | 118 | 20.133 | 22.575 | 52.422 | 1.00 | 17.95 |
| ATOM | 914 | O | VAL | 118 | 21.338 | 22.351 | 52.430 | 1.00 | 17.23 |
| ATOM | 915 | N | ALA | 119 | 19.326 | 22.435 | 53.478 | 1.00 | 17.06 |
| ATOM | 916 | CA | ALA | 119 | 19.833 | 21.907 | 54.727 | 1.00 | 17.10 |
| ATOM | 917 | CB | ALA | 119 | 18.932 | 22.450 | 55.851 | 1.00 | 17.38 |
| ATOM | 918 | C | ALA | 119 | 19.739 | 20.368 | 54.722 | 1.00 | 16.72 |
| ATOM | 919 | O | ALA | 119 | 18.760 | 19.858 | 54.161 | 1.00 | 16.13 |
| ATOM | 920 | N | GLY | 120 | 20.718 | 19.683 | 55.320 | 1.00 | 15.41 |
| ATOM | 921 | CA | GLY | 120 | 20.621 | 18.216 | 55.347 | 1.00 | 14.28 |
| ATOM | 922 | C | GLY | 120 | 21.960 | 17.576 | 55.058 | 1.00 | 13.76 |
| ATOM | 923 | O | GLY | 120 | 22.737 | 18.067 | 54.234 | 1.00 | 14.66 |
| ATOM | 924 | N | LEU | 121 | 22.244 | 16.452 | 55.717 | 1.00 | 12.70 |
| ATOM | 925 | CA | LEU | 121 | 23.529 | 15.767 | 55.522 | 1.00 | 11.00 |
| ATOM | 926 | CB | LEU | 121 | 23.882 | 15.004 | 56.781 | 1.00 | 10.76 |
| ATOM | 927 | CG | LEU | 121 | 23.943 | 15.759 | 58.103 | 1.00 | 9.49 |
| ATOM | 928 | CD1 | LEU | 121 | 24.449 | 14.822 | 59.193 | 1.00 | 7.07 |
| ATOM | 929 | CD2 | LEU | 121 | 24.866 | 16.971 | 57.969 | 1.00 | 8.49 |
| ATOM | 930 | C | LEU | 121 | 23.528 | 14.851 | 54.310 | 1.00 | 9.73 |
| ATOM | 931 | O | LEU | 121 | 22.575 | 14.134 | 53.975 | 1.00 | 10.55 |
| ATOM | 932 | N | VAL | 122 | 24.614 | 14.916 | 53.575 | 1.00 | 9.32 |
| ATOM | 933 | CA | VAL | 122 | 24.834 | 14.130 | 52.353 | 1.00 | 8.52 |
| ATOM | 934 | CB | VAL | 122 | 25.063 | 15.033 | 51.127 | 1.00 | 6.54 |
| ATOM | 935 | CG1 | VAL | 122 | 25.255 | 14.183 | 49.858 | 1.00 | 6.34 |
| ATOM | 936 | CG2 | VAL | 122 | 23.909 | 15.992 | 50.882 | 1.00 | 7.19 |
| ATOM | 937 | C | VAL | 122 | 26.025 | 13.196 | 52.613 | 1.00 | 8.38 |
| ATOM | 938 | O | VAL | 122 | 27.194 | 13.572 | 52.554 | 1.00 | 7.49 |
| ATOM | 939 | N | HIS | 123 | 25.693 | 11.961 | 52.933 | 1.00 | 8.70 |
| ATOM | 940 | CA | HIS | 123 | 26.619 | 10.910 | 53.254 | 1.00 | 9.15 |
| ATOM | 941 | CB | HIS | 123 | 25.995 | 9.8635 | 54.141 | 1.00 | 8.55 |
| ATOM | 942 | CG | HIS | 123 | 25.490 | 10.280 | 55.476 | 1.00 | 8.68 |
| ATOM | 943 | ND1 | HIS | 123 | 24.164 | 10.635 | 55.678 | 1.00 | 8.01 |
| ATOM | 944 | CE1 | HIS | 123 | 23.986 | 10.913 | 56.974 | 1.00 | 7.33 |
| ATOM | 945 | NE2 | HIS | 123 | 25.156 | 10.789 | 57.597 | 1.00 | 9.84 |
| ATOM | 946 | CD2 | HIS | 123 | 26.079 | 10.374 | 56.687 | 1.00 | 8.51 |
| ATOM | 947 | C | HIS | 123 | 27.238 | 10.273 | 51.992 | 1.00 | 8.74 |
| ATOM | 948 | O | HIS | 123 | 26.548 | 9.8185 | 51.133 | 1.00 | 9.79 |
| ATOM | 949 | N | ARG | 124 | 28.544 | 10.295 | 51.908 | 1.00 | 8.30 |
| ATOM | 950 | CA | ARG | 124 | 29.327 | 9.8085 | 50.787 | 1.00 | 7.51 |
| ATOM | 951 | CB | ARG | 124 | 29.950 | 11.016 | 50.038 | 1.00 | 6.35 |
| ATOM | 952 | CG | ARG | 124 | 28.857 | 11.950 | 49.441 | 1.00 | 6.41 |
| ATOM | 953 | CD | ARG | 124 | 29.525 | 12.993 | 48.565 | 1.00 | 6.17 |
| ATOM | 954 | NE | ARG | 124 | 28.611 | 13.914 | 47.957 | 1.00 | 6.87 |
| ATOM | 955 | CZ | ARG | 124 | 27.747 | 13.824 | 46.996 | 1.00 | 8.12 |
| ATOM | 956 | NH1 | ARG | 124 | 27.516 | 12.728 | 46.252 | 1.00 | 6.27 |
| ATOM | 957 | NH2 | ARG | 124 | 27.033 | 14.909 | 46.631 | 1.00 | 7.84 |
| ATOM | 958 | C | ARG | 124 | 30.408 | 8.871 | 51.264 | 1.00 | 7.13 |
| ATOM | 959 | O | ARG | 124 | 30.496 | 8.686 | 52.482 | 1.00 | 7.16 |
| ATOM | 960 | N | HIS | 125 | 31.238 | 8.287 | 50.388 | 1.00 | 8.38 |
| ATOM | 961 | CA | HIS | 125 | 32.302 | 7.401 | 50.830 | 1.00 | 9.33 |
| ATOM | 962 | CB | HIS | 125 | 32.816 | 6.389 | 49.794 | 1.00 | 9.69 |
| ATOM | 963 | CG | HIS | 125 | 31.660 | 5.665 | 49.141 | 1.00 | 7.74 |
| ATOM | 964 | ND1 | HIS | 125 | 30.806 | 4.831 | 49.778 | 1.00 | 8.49 |
| ATOM | 965 | CE1 | HIS | 125 | 29.907 | 4.383 | 48.933 | 1.00 | 7.96 |
| ATOM | 966 | NE2 | HIS | 125 | 30.208 | 4.908 | 47.725 | 1.00 | 9.47 |
| ATOM | 967 | CD2 | HIS | 125 | 31.289 | 5.714 | 47.836 | 1.00 | 8.48 |
| ATOM | 968 | C | HIS | 125 | 33.495 | 8.241 | 51.292 | 1.00 | 9.98 |
| ATOM | 969 | O | HIS | 125 | 33.868 | 9.228 | 50.668 | 1.00 | 11.12 |
| ATOM | 970 | N | ASN | 126 | 34.083 | 7.837 | 52.420 | 1.00 | 8.74 |
| ATOM | 971 | CA | ASN | 126 | 35.193 | 8.623 | 52.953 | 1.00 | 8.54 |
| ATOM | 972 | CB | ASN | 126 | 35.428 | 8.115 | 54.411 | 1.00 | 8.14 |
| ATOM | 973 | CG | ASN | 126 | 36.416 | 9.019 | 55.101 | 1.00 | 8.88 |
| ATOM | 974 | OD1 | ASN | 126 | 36.270 | 10.238 | 54.953 | 1.00 | 10.54 |
| ATOM | 975 | ND2 | ASN | 126 | 37.448 | 8.494 | 55.731 | 1.00 | 9.56 |
| ATOM | 976 | C | ASN | 126 | 36.402 | 8.566 | 52.090 | 1.00 | 8.32 |
| ATOM | 977 | O | ASN | 126 | 37.199 | 9.515 | 51.941 | 1.00 | 9.13 |
| ATOM | 978 | N | LYS | 127 | 36.595 | 7.416 | 51.408 | 1.00 | 8.46 |
| ATOM | 979 | CA | LYS | 127 | 37.733 | 7.182 | 50.536 | 1.00 | 6.93 |
| ATOM | 980 | CB | LYS | 127 | 38.785 | 6.232 | 51.138 | 1.00 | 8.23 |
| ATOM | 981 | CG | LYS | 127 | 39.335 | 6.671 | 52.443 | 1.00 | 13.12 |
| ATOM | 982 | CD | LYS | 127 | 40.489 | 5.811 | 52.965 | 1.00 | 16.68 |
| ATOM | 983 | CE | LYS | 127 | 40.871 | 6.388 | 54.335 | 1.00 | 21.82 |
| ATOM | 984 | NZ | LYS | 127 | 41.984 | 5.677 | 54.991 | 1.00 | 26.43 |
| ATOM | 985 | C | LYS | 127 | 37.279 | 6.566 | 49.206 | 1.00 | 5.67 |
| ATOM | 986 | O | LYS | 127 | 36.408 | 5.710 | 49.137 | 1.00 | 5.09 |
| ATOM | 987 | N | ILE | 128 | 37.890 | 7.084 | 48.137 | 1.00 | 6.06 |
| ATOM | 988 | CA | ILE | 128 | 37.584 | 6.599 | 46.802 | 1.00 | 5.35 |
| ATOM | 989 | CB | ILE | 128 | 36.554 | 7.482 | 46.060 | 1.00 | 3.79 |
| ATOM | 990 | CG1 | ILE | 128 | 37.101 | 8.916 | 45.871 | 1.00 | 2.49 |
| ATOM | 991 | CD1 | ILE | 128 | 36.165 | 9.779 | 45.033 | 1.00 | 4.28 |
| ATOM | 992 | CG2 | ILE | 128 | 35.180 | 7.503 | 46.769 | 1.00 | 2.00 |
| ATOM | 993 | C | ILE | 128 | 38.855 | 6.553 | 45.951 | 1.00 | 4.84 |
| ATOM | 994 | O | ILE | 128 | 39.849 | 7.235 | 46.194 | 1.00 | 3.72 |
| ATOM | 995 | N | THR | 129 | 38.812 | 5.755 | 44.906 | 1.00 | 4.26 |
| ATOM | 996 | CA | THR | 129 | 39.886 | 5.639 | 43.932 | 1.00 | 5.44 |
| ATOM | 997 | CB THR | 129 | 40.600 | 4.262 | 43.874 | 1.00 | 5.74 | |
| ATOM | 998 | OG1 THR | 129 | 41.384 | 4.124 | 45.083 | 1.00 | 6.67 | |
| ATOM | 999 | CG2 THR | 129 | 41.546 | 4.171 | 42.697 | 1.00 | 3.87 | |
| ATOM | 1000 | C | THR | 129 | 39.130 | 5.882 | 42.610 | 1.00 | 5.76 |
| ATOM | 1001 | O | THR | 129 | 38.104 | 5.237 | 42.387 | 1.00 | 4.96 |
| ATOM | 1002 | N | ILE | 130 | 39.584 | 6.856 | 41.882 | 1.00 | 6.40 |
| ATOM | 1003 | CA | ILE | 130 | 39.076 | 7.252 | 40.599 | 1.00 | 7.37 |
| ATOM | 1004 | CB | ILE | 130 | 38.692 | 8.769 | 40.544 | 1.00 | 7.11 |
| ATOM | 1005 | CG1 | ILE | 130 | 37.391 | 8.969 | 41.370 | 1.00 | 7.19 |
| ATOM | 1006 | CD1 | ILE | 130 | 36.998 | 10.444 | 41.506 | 1.00 | 10.30 |
| ATOM | 1007 | CG2 | ILE | 130 | 38.474 | 9.320 | 39.150 | 1.00 | 7.49 |
| ATOM | 1008 | C | ILE | 130 | 40.132 | 7.022 | 39.492 | 1.00 | 7.21 |
| ATOM | 1009 | O | ILE | 130 | 41.291 | 7.322 | 39.617 | 1.00 | 6.63 |
| ATOM | 1010 | N | LYS | 131 | 39.603 | 6.521 | 38.372 | 1.00 | 7.02 |
| ATOM | 1011 | CA | LYS | 131 | 40.359 | 6.352 | 37.170 | 1.00 | 8.19 |
| ATOM | 1012 | CB | LYS | 131 | 40.513 | 4.982 | 36.602 | 1.00 | 11.33 |
| ATOM | 1013 | CG | LYS | 131 | 41.314 | 3.971 | 37.416 | 1.00 | 13.57 |
| ATOM | 1014 | CD | LYS | 131 | 41.458 | 2.749 | 36.505 | 1.00 | 17.15 |
| ATOM | 1015 | CE | LYS | 131 | 42.455 | 1.746 | 37.039 | 1.00 | 20.87 |
| ATOM | 1016 | NZ | LYS | 131 | 42.779 | 0.788 | 35.929 | 1.00 | 25.22 |
| ATOM | 1017 | C | LYS | 131 | 39.709 | 7.288 | 36.109 | 1.00 | 6.91 |
| ATOM | 1018 | O | LYS | 131 | 38.489 | 7.342 | 36.009 | 1.00 | 5.90 |
| ATOM | 1019 | N | ALA | 132 | 40.583 | 8.053 | 35.447 | 1.00 | 6.42 |
| ATOM | 1020 | CA | ALA | 132 | 40.013 | 8.945 | 34.400 | 1.00 | 5.33 |
| ATOM | 1021 | CB | ALA | 132 | 39.562 | 10.235 | 35.083 | 1.00 | 2.00 |
| ATOM | 1022 | C | ALA | 132 | 41.063 | 9.240 | 33.354 | 1.00 | 5.57 |
| ATOM | 1023 | O | ALA | 132 | 42.191 | 8.728 | 33.449 | 1.00 | 6.67 |
| ATOM | 1024 | N | LYS | 133 | 40.734 | 10.026 | 32.341 | 1.00 | 6.83 |
| ATOM | 1025 | CA | LYS | 133 | 41.758 | 10.489 | 31.375 | 1.00 | 7.84 |
| ATOM | 1026 | CB | LYS | 133 | 41.311 | 10.463 | 29.904 | 1.00 | 9.83 |
| ATOM | 1027 | CG | LYS | 133 | 40.878 | 9.164 | 29.336 | 1.00 | 11.29 |
| ATOM | 1028 | CD | LYS | 133 | 41.983 | 8.133 | 29.442 | 1.00 | 14.36 |
| ATOM | 1029 | CE | LYS | 133 | 41.577 | 6.842 | 28.727 | 1.00 | 16.88 |
| ATOM | 1030 | NZ | LYS | 133 | 42.601 | 5.811 | 29.133 | 1.00 | 17.94 |
| ATOM | 1031 | C | LYS | 133 | 41.887 | 12.006 | 31.643 | 1.00 | 6.97 |
| ATOM | 1032 | O | LYS | 133 | 40.940 | 12.600 | 32.143 | 1.00 | 7.81 |
| ATOM | 1033 | N | ASP | 134 | 42.949 | 12.623 | 31.268 | 1.00 | 8.29 |
| ATOM | 1034 | CA | ASP | 134 | 43.141 | 14.055 | 31.363 | 1.00 | 7.39 |
| ATOM | 1035 | CB | ASP | 134 | 44.519 | 14.454 | 31.823 | 1.00 | 8.51 |
| ATOM | 1036 | CG | ASP | 134 | 45.717 | 14.189 | 30.975 | 1.00 | 8.37 |
| ATOM | 1037 | OD1 | ASP | 134 | 46.894 | 14.220 | 31.413 | 1.00 | 8.77 |
| ATOM | 1038 | OD2 | ASP | 134 | 45.585 | 13.946 | 29.746 | 1.00 | 9.62 |
| ATOM | 1039 | C | ASP | 134 | 42.752 | 14.675 | 30.015 | 1.00 | 9.13 |
| ATOM | 1040 | O | ASP | 134 | 42.283 | 13.980 | 29.053 | 1.00 | 10.00 |
| ATOM | 1041 | N | ILE | 135 | 42.905 | 15.962 | 29.856 | 1.00 | 8.82 |
| ATOM | 1042 | CA | ILE | 135 | 42.538 | 16.710 | 28.635 | 1.00 | 9.79 |
| ATOM | 1043 | CB | ILE | 135 | 42.652 | 18.224 | 28.949 | 1.00 | 10.08 |
| ATOM | 1044 | CG1 | ILE | 135 | 41.900 | 19.058 | 27.938 | 1.00 | 9.25 |
| ATOM | 1045 | CD1 | ILE | 135 | 41.972 | 20.551 | 28.227 | 1.00 | 9.86 |
| ATOM | 1046 | CG2 | ILE | 135 | 44.134 | 18.642 | 28.921 | 1.00 | 10.69 |
| ATOM | 1047 | C | ILE | 135 | 43.229 | 16.263 | 27.395 | 1.00 | 9.88 |
| ATOM | 1048 | O | ILE | 135 | 42.729 | 16.310 | 26.254 | 1.00 | 10.08 |
| ATOM | 1049 | N | GLU | 136 | 44.447 | 15.717 | 27.490 | 1.00 | 9.82 |
| ATOM | 1050 | CA | GLU | 136 | 45.211 | 15.168 | 26.423 | 1.00 | 9.97 |
| ATOM | 1051 | CB | GLU | 136 | 46.721 | 15.501 | 26.503 | 1.00 | 9.18 |
| ATOM | 1052 | CG | GLU | 136 | 47.025 | 16.963 | 26.269 | 1.00 | 11.11 |
| ATOM | 1053 | CD | GLU | 136 | 46.573 | 17.426 | 24.871 | 1.00 | 11.43 |
| ATOM | 1054 | OE1 | GLU | 136 | 46.759 | 16.636 | 23.938 | 1.00 | 12.83 |
| ATOM | 1055 | OE2 | GLU | 136 | 46.039 | 18.527 | 24.823 | 1.00 | 12.16 |
| ATOM | 1056 | C | GLU | 136 | 45.033 | 13.647 | 26.240 | 1.00 | 9.67 |
| ATOM | 1057 | O | GLU | 136 | 45.793 | 13.083 | 25.431 | 1.00 | 10.16 |
| ATOM | 1058 | N | GLY | 137 | 44.149 | 12.996 | 26.990 | 1.00 | 8.60 |
| ATOM | 1059 | CA | GLY | 137 | 43.916 | 11.554 | 26.862 | 1.00 | 7.80 |
| ATOM | 1060 | C | GLY | 137 | 44.812 | 10.661 | 27.640 | 1.00 | 6.10 |
| ATOM | 1061 | O | GLY | 137 | 44.951 | 9.443 | 27.445 | 1.00 | 6.27 |
| ATOM | 1062 | N | ASN | 138 | 45.611 | 11.224 | 28.533 | 1.00 | 6.53 |
| ATOM | 1063 | CA | ASN | 138 | 46.540 | 10.485 | 29.348 | 1.00 | 7.17 |
| ATOM | 1064 | CB | ASN | 138 | 47.859 | 11.112 | 29.564 | 1.00 | 6.62 |
| ATOM | 1065 | CG | ASN | 138 | 48.666 | 11.343 | 28.324 | 1.00 | 6.41 |
| ATOM | 1066 | OD1 | ASN | 138 | 48.891 | 10.474 | 27.473 | 1.00 | 7.83 |
| ATOM | 1067 | ND2 | ASN | 138 | 49.080 | 12.589 | 28.118 | 1.00 | 5.84 |
| ATOM | 1068 | C | ASN | 138 | 45.782 | 9.938 | 30.557 | 1.00 | 8.75 |
| ATOM | 1069 | O | ASN | 138 | 44.731 | 10.431 | 30.900 | 1.00 | 9.34 |
| ATOM | 1070 | N | ASP | 139 | 46.289 | 8.893 | 31.200 | 1.00 | 9.51 |
| ATOM | 1071 | CA | ASP | 139 | 45.642 | 8.232 | 32.304 | 1.00 | 9.46 |
| ATOM | 1072 | CB | ASP | 139 | 46.137 | 6.738 | 32.368 | 1.00 | 9.74 |
| ATOM | 1073 | CG | ASP | 139 | 45.953 | 6.042 | 31.034 | 1.00 | 10.25 |
| ATOM | 1074 | OD1 | ASP | 139 | 45.029 | 6.416 | 30.295 | 1.00 | 10.01 |
| ATOM | 1075 | OD2 | ASP | 139 | 46.802 | 5.192 | 30.714 | 1.00 | 10.42 |
| ATOM | 1076 | C | ASP | 139 | 45.931 | 8.866 | 33.648 | 1.00 | 8.48 |
| ATOM | 1077 | O | ASP | 139 | 47.094 | 9.212 | 33.934 | 1.00 | 8.89 |
| ATOM | 1078 | N | ILE | 140 | 44.896 | 9.015 | 34.458 | 1.00 | 8.07 |
| ATOM | 1079 | CA | ILE | 140 | 45.072 | 9.590 | 35.799 | 1.00 | 9.43 |
| ATOM | 1080 | CB | ILE | 140 | 44.435 | 10.979 | 35.964 | 1.00 | 12.18 |
| ATOM | 1081 | CG1 | ILE | 140 | 44.723 | 11.979 | 34.883 | 1.00 | 15.73 |
| ATOM | 1082 | CD1 | ILE | 140 | 46.188 | 12.373 | 34.669 | 1.00 | 17.79 |
| ATOM | 1083 | CG2 | ILE | 140 | 44.881 | 11.612 | 37.311 | 1.00 | 13.76 |
| ATOM | 1084 | C | ILE | 140 | 44.358 | 8.632 | 36.775 | 1.00 | 8.12 |
| ATOM | 1085 | O | ILE | 140 | 43.265 | 8.238 | 36.565 | 1.00 | 7.55 |
| ATOM | 1086 | N | GLN | 141 | 44.992 | 8.330 | 37.908 | 1.00 | 8.68 |
| ATOM | 1087 | CA | GLN | 141 | 44.370 | 7.492 | 38.929 | 1.00 | 8.09 |
| ATOM | 1088 | CB | GLN | 141 | 44.930 | 6.081 | 39.016 | 1.00 | 10.14 |
| ATOM | 1089 | CG | CLN | 141 | 44.139 | 5.210 | 39.984 | 1.00 | 13.25 |
| ATOM | 1090 | CD | GLN | 141 | 44.556 | 3.767 | 40.096 | 1.00 | 14.70 |
| ATOM | 1091 | OE1 | GLN | 141 | 45.723 | 3.425 | 40.103 | 1.00 | 16.12 |
| ATOM | 1092 | NE2 | GLN | 141 | 43.607 | 2.851 | 40.176 | 1.00 | 14.82 |
| ATOM | 1093 | C | GLN | 141 | 44.575 | 8.281 | 40.247 | 1.00 | 6.14 |
| ATOM | 1094 | O | GLN | 141 | 45.713 | 8.595 | 40.573 | 1.00 | 7.17 |
| ATOM | 1095 | N | LEU | 142 | 43.498 | 8.589 | 40.928 | 1.00 | 4.24 |
| ATOM | 1096 | CA | LEU | 142 | 43.540 | 9.376 | 42.127 | 1.00 | 4.16 |
| ATOM | 1097 | CB | LEU | 142 | 42.641 | 10.638 | 42.092 | 1.00 | 3.14 |
| ATOM | 1098 | CG | LEU | 142 | 42.865 | 11.671 | 40.964 | 1.00 | 4.72 |
| ATOM | 1099 | CD1 | LEU | 142 | 42.418 | 11.139 | 39.628 | 1.00 | 7.12 |
| ATOM | 1100 | CD2 | LEU | 142 | 42.191 | 12.984 | 41.303 | 1.00 | 5.38 |
| ATOM | 1101 | C | LEU | 142 | 43.024 | 8.590 | 43.362 | 1.00 | 4.55 |
| ATOM | 1102 | O | LEU | 142 | 42.026 | 7.918 | 43.302 | 1.00 | 3.43 |
| ATOM | 1103 | N | ARG | 143 | 43.706 | 8.838 | 44.442 | 1.00 | 5.60 |
| ATOM | 1104 | CA | ARG | 143 | 43.254 | 8.230 | 45.735 | 1.00 | 6.45 |
| ATOM | 1105 | CB | ARG | 143 | 44.406 | 7.410 | 46.295 | 1.00 | 6.30 |
| ATOM | 1106 | CG | ARG | 143 | 44.572 | 6.089 | 45.510 | 1.00 | 6.38 |
| ATOM | 1107 | CD | ARG | 143 | 45.870 | 5.421 | 45.955 | 1.00 | 7.24 |
| ATOM | 1108 | NE | ARG | 143 | 46.994 | 6.164 | 45.399 | 1.00 | 8.41 |
| ATOM | 1109 | CZ | ARG | 143 | 47.487 | 6.175 | 44.186 | 1.00 | 7.10 |
| ATOM | 1110 | NH1 | ARG | 143 | 47.010 | 5.371 | 43.229 | 1.00 | 10.56 |
| ATOM | 1111 | NH2 | ARG | 143 | 48.441 | 6.994 | 43.872 | 1.00 | 6.66 |
| ATOM | 1112 | C | ARG | 143 | 42.862 | 9.416 | 46.587 | 1.00 | 6.59 |
| ATOM | 1113 | O | ARG | 143 | 43.759 | 10.213 | 46.872 | 1.00 | 8.32 |
| ATOM | 1114 | N | LEU | 144 | 41.608 | 9.616 | 46.887 | 1.00 | 6.29 |
| ATOM | 1115 | CA | LEU | 144 | 41.146 | 10.800 | 47.621 | 1.00 | 7.40 |
| ATOM | 1116 | CB | LEU | 144 | 40.077 | 11.501 | 46.737 | 1.00 | 8.55 |
| ATOM | 1117 | CG | LEU | 144 | 40.603 | 12.027 | 45.375 | 1.00 | 8.47 |
| ATOM | 1118 | CD1 | LEU | 144 | 39.402 | 12.282 | 44.481 | 1.00 | 6.95 |
| ATOM | 1119 | CD2 | LEU | 144 | 41.379 | 13.312 | 45.591 | 1.00 | 5.70 |
| ATOM | 1120 | C | LEU | 144 | 40.470 | 10.405 | 48.909 | 1.00 | 9.13 |
| ATOM | 1121 | O | LEU | 144 | 39.953 | 9.293 | 49.040 | 1.00 | 8.94 |
| ATOM | 1122 | N | LYS | 145 | 40.423 | 11.365 | 49.832 | 1.00 | 10.82 |
| ATOM | 1123 | CA | LYS | 145 | 39.801 | 11.116 | 51.142 | 1.00 | 11.39 |
| ATOM | 1124 | CB | LYS | 145 | 40.924 | 10.619 | 52.041 | 1.00 | 13.95 |
| ATOM | 1125 | CG | LYS | 145 | 40.646 | 10.374 | 53.458 | 1.00 | 18.80 |
| ATOM | 1126 | CD | LYS | 145 | 41.861 | 9.909 | 54.232 | 1.00 | 25.06 |
| ATOM | 1127 | CE | LYS | 145 | 42.963 | 10.908 | 54.354 | 1.00 | 31.66 |
| ATOM | 1128 | NZ | LYS | 145 | 43.539 | 11.396 | 53.059 | 1.00 | 37.64 |
| ATOM | 1129 | C | LYS | 145 | 39.188 | 12.388 | 51.720 | 1.00 | 9.96 |
| ATOM | 1130 | O | LYS | 145 | 39.596 | 13.478 | 51.389 | 1.00 | 9.17 |
| ATOM | 1131 | N | GLY | 146 | 38.108 | 12.225 | 52.519 | 1.00 | 8.79 |
| ATOM | 1132 | CA | GLY | 146 | 37.456 | 13.328 | 53.137 | 1.00 | 8.43 |
| ATOM | 1133 | C | GLY | 146 | 36.897 | 14.360 | 52.201 | 1.00 | 8.40 |
| ATOM | 1134 | O | GLY | 146 | 36.151 | 14.060 | 51.254 | 1.00 | 8.45 |
| ATOM | 1135 | N | TYR | 147 | 37.263 | 15.616 | 52.405 | 1.00 | 8.09 |
| ATOM | 1136 | CA | TYR | 147 | 36.779 | 16.715 | 51.642 | 1.00 | 7.87 |
| ATOM | 1137 | CB | TYR | 147 | 37.196 | 18.074 | 52.234 | 1.00 | 9.36 |
| ATOM | 1138 | CG | TYR | 147 | 36.595 | 19.261 | 51.474 | 1.00 | 11.07 |
| ATOM | 1139 | CD1 | TYR | 147 | 35.277 | 19.634 | 51.672 | 1.00 | 11.07 |
| ATOM | 1140 | CE1 | TYR | 147 | 34.748 | 20.679 | 50.935 | 1.00 | 13.45 |
| ATOM | 1141 | CZ | TYR | 147 | 35.529 | 21.400 | 50.057 | 1.00 | 13.62 |
| ATOM | 1142 | OH | TYR | 147 | 35.036 | 22.483 | 49.389 | 1.00 | 13.97 |
| ATOM | 1143 | CE2 | TYR | 147 | 36.866 | 21.045 | 49.873 | 1.00 | 13.40 |
| ATOM | 1144 | CD2 | TYR | 147 | 37.362 | 19.974 | 50.592 | 1.00 | 12.10 |
| ATOM | 1145 | C | TYR | 147 | 36.885 | 16.626 | 50.152 | 1.00 | 5.90 |
| ATOM | 1146 | O | TYR | 147 | 35.845 | 16.786 | 49.504 | 1.00 | 4.84 |
| ATOM | 1147 | N | PRO | 148 | 38.043 | 16.403 | 49.590 | 1.00 | 7.00 |
| ATOM | 1148 | CA | PRO | 148 | 38.156 | 16.303 | 48.132 | 1.00 | 7.78 |
| ATOM | 1149 | CB | PRO | 148 | 39.605 | 16.180 | 47.837 | 1.00 | 7.31 |
| ATOM | 1150 | CG | PRO | 148 | 40.271 | 15.930 | 49.125 | 1.00 | 8.35 |
| ATOM | 1151 | CD | PRO | 148 | 39.340 | 16.286 | 50.255 | 1.00 | 7.21 |
| ATOM | 1152 | C | PRO | 148 | 37.394 | 15.074 | 47.639 | 1.00 | 7.01 |
| ATOM | 1153 | O | PRO | 148 | 36.995 | 15.119 | 46.479 | 1.00 | 8.08 |
| ATOM | 1154 | N | ALA | 149 | 37.271 | 14.025 | 48.435 | 1.00 | 5.19 |
| ATOM | 1155 | CA | ALA | 149 | 36.571 | 12.828 | 48.000 | 1.00 | 4.31 |
| ATOM | 1156 | CB | ALA | 149 | 36.715 | 11.666 | 48.983 | 1.00 | 2.00 |
| ATOM | 1157 | C | ALA | 149 | 35.079 | 13.175 | 47.824 | 1.00 | 3.82 |
| ATOM | 1158 | O | ALA | 149 | 34.463 | 12.772 | 46.850 | 1.00 | 3.79 |
| ATOM | 1159 | N | ILE | 150 | 34.535 | 13.957 | 48.742 | 1.00 | 4.49 |
| ATOM | 1160 | CA | ILE | 150 | 33.146 | 14.400 | 48.705 | 1.00 | 5.37 |
| ATOM | 1161 | CB | ILE | 150 | 32.790 | 15.107 | 50.026 | 1.00 | 7.40 |
| ATOM | 1162 | CG2 | ILE | 150 | 31.624 | 16.049 | 49.837 | 1.00 | 7.23 |
| ATOM | 1163 | CG1 | ILE | 150 | 32.660 | 14.085 | 51.137 | 1.00 | 8.06 |
| ATOM | 1164 | CD1 | ILE | 150 | 32.623 | 14.512 | 52.578 | 1.00 | 7.31 |
| ATOM | 1165 | C | ILE | 150 | 32.925 | 15.337 | 47.504 | 1.00 | 4.53 |
| ATOM | 1166 | O | ILE | 150 | 31.943 | 15.147 | 46.813 | 1.00 | 3.53 |
| ATOM | 1167 | N | VAL | 151 | 33.896 | 16.213 | 47.249 | 1.00 | 4.55 |
| ATOM | 1168 | CA | VAL | 151 | 33.781 | 17.130 | 46.083 | 1.00 | 4.71 |
| ATOM | 1169 | CB | VAL | 151 | 34.800 | 18.238 | 46.088 | 1.00 | 4.83 |
| ATOM | 1170 | CG1 | VAL | 151 | 34.777 | 19.142 | 44.820 | 1.00 | 4.32 |
| ATOM | 1171 | CG2 | VAL | 151 | 34.475 | 19.164 | 47.311 | 1.00 | 5.52 |
| ATOM | 1172 | C | VAL | 151 | 33.820 | 16.304 | 44.795 | 1.00 | 5.66 |
| ATOM | 1173 | O | VAL | 151 | 32.961 | 16.581 | 43.957 | 1.00 | 6.35 |
| ATOM | 1174 | N | PHE | 152 | 34.749 | 15.379 | 44.629 | 1.00 | 4.24 |
| ATOM | 1175 | CA | PHE | 152 | 34.658 | 14.562 | 43.361 | 1.00 | 4.96 |
| ATOM | 1176 | CB | PHE | 152 | 35.902 | 13.806 | 43.085 | 1.00 | 4.05 |
| ATOM | 1177 | CG | PHE | 152 | 37.140 | 14.561 | 42.669 | 1.00 | 6.55 |
| ATOM | 1178 | CD1 | PHE | 152 | 37.911 | 15.246 | 43.583 | 1.00 | 7.52 |
| ATOM | 1179 | CE1 | PHE | 152 | 39.104 | 15.892 | 43.234 | 1.00 | 6.30 |
| ATOM | 1180 | CZ | PHE | 152 | 39.474 | 15.858 | 41.904 | 1.00 | 4.29 |
| ATOM | 1181 | CE2 | PHE | 152 | 38.732 | 15.203 | 40.973 | 1.00 | 3.38 |
| ATOM | 1182 | CD2 | PHE | 152 | 37.586 | 14.528 | 41.355 | 1.00 | 4.89 |
| ATOM | 1183 | C | PHE | 152 | 33.386 | 13.735 | 43.308 | 1.00 | 4.69 |
| ATOM | 1184 | O | PHE | 152 | 32.812 | 13.579 | 42.203 | 1.00 | 5.23 |
| ATOM | 1185 | N | GLN | 153 | 32.847 | 13.159 | 44.374 | 1.00 | 4.19 |
| ATOM | 1186 | CA | GLN | 153 | 31.588 | 12.395 | 44.269 | 1.00 | 5.10 |
| ATOM | 1187 | CB | GLN | 153 | 31.284 | 11.638 | 45.555 | 1.00 | 4.46 |
| ATOM | 1188 | CG | GLN | 153 | 32.324 | 10.564 | 45.854 | 1.00 | 5.68 |
| ATOM | 1189 | CD | GLN | 153 | 32.214 | 9.837 | 47.177 | 1.00 | 7.54 |
| ATOM | 1190 | OE1 | GLN | 153 | 31.475 | 8.874 | 47.401 | 1.00 | 9.94 |
| ATOM | 1191 | NE2 | GLN | 153 | 33.031 | 10.289 | 48.117 | 1.00 | 5.48 |
| ATOM | 1192 | C | GLN | 153 | 30.446 | 13.259 | 43.812 | 1.00 | 5.17 |
| ATOM | 1193 | O | GLN | 153 | 29.581 | 12.883 | 42.981 | 1.00 | 6.71 |
| ATOM | 1194 | N | HIS | 154 | 30.385 | 14.471 | 44.338 | 1.00 | 4.09 |
| ATOM | 1195 | CA | HIS | 154 | 29.340 | 15.431 | 43.990 | 1.00 | 4.25 |
| ATOM | 1196 | CB | HIS | 154 | 29.636 | 16.731 | 44.863 | 1.00 | 3.87 |
| ATOM | 1197 | CG | HIS | 154 | 28.693 | 17.851 | 44.588 | 1.00 | 3.28 |
| ATOM | 1198 | ND1 | HIS | 154 | 27.711 | 18.274 | 45.436 | 1.00 | 5.34 |
| ATOM | 1199 | CE1 | HIS | 154 | 27.004 | 19.245 | 44.925 | 1.00 | 4.80 |
| ATOM | 1200 | NE2 | HIS | 154 | 27.491 | 19.463 | 43.683 | 1.00 | 6.11 |
| ATOM | 1201 | CD2 | HIS | 154 | 28.529 | 18.603 | 43.465 | 1.00 | 5.20 |
| ATOM | 1202 | C | HIS | 154 | 29.391 | 15.728 | 42.477 | 1.00 | 3.83 |
| ATOM | 1203 | O | HIS | 154 | 28.401 | 15.710 | 41.793 | 1.00 | 3.56 |
| ATOM | 1204 | N | GLU | 155 | 30.571 | 16.019 | 41.951 | 1.00 | 4.91 |
| ATOM | 1205 | CA | GLU | 155 | 30.779 | 16.302 | 40.553 | 1.00 | 7.09 |
| ATOM | 1206 | CB | GLU | 155 | 32.150 | 16.925 | 40.264 | 1.00 | 7.65 |
| ATOM | 1207 | CG | GLU | 155 | 32.331 | 18.281 | 41.029 | 1.00 | 6.88 |
| ATOM | 1208 | CD | GLU | 155 | 31.161 | 19.223 | 40.725 | 1.00 | 8.48 |
| ATOM | 1209 | OE1 | GLU | 155 | 30.644 | 19.235 | 39.570 | 1.00 | 6.97 |
| ATOM | 1210 | OE2 | GLU | 155 | 30.704 | 19.907 | 41.673 | 1.00 | 9.81 |
| ATOM | 1211 | C | GLU | 155 | 30.481 | 15.079 | 39.675 | 1.00 | 6.80 |
| ATOM | 1212 | O | GLU | 155 | 29.815 | 15.254 | 38.625 | 1.00 | 7.83 |
| ATOM | 1213 | N | ILE | 156 | 30.914 | 13.904 | 40.026 | 1.00 | 6.49 |
| ATOM | 1214 | CA | ILE | 156 | 30.573 | 12.698 | 39.226 | 1.00 | 6.27 |
| ATOM | 1215 | CB | ILE | 156 | 31.359 | 11.470 | 39.668 | 1.00 | 5.97 |
| ATOM | 1216 | CG1 | ILE | 156 | 32.848 | 11.653 | 39.436 | 1.00 | 4.24 |
| ATOM | 1217 | CD1 | ILE | 156 | 33.760 | 10.571 | 39.861 | 1.00 | 3.82 |
| ATOM | 1218 | CG2 | ILE | 156 | 30.804 | 10.186 | 39.036 | 1.00 | 7.37 |
| ATOM | 1219 | C | ILE | 156 | 29.066 | 12.513 | 39.298 | 1.00 | 5.98 |
| ATOM | 1220 | O | ILE | 156 | 28.467 | 12.196 | 38.252 | 1.00 | 5.77 |
| ATOM | 1221 | N | ASP | 157 | 28.408 | 12.644 | 40.429 | 1.00 | 5.23 |
| ATOM | 1222 | CA | ASP | 157 | 26.978 | 12.553 | 40.507 | 1.00 | 5.85 |
| ATOM | 1223 | CB | ASP | 157 | 26.371 | 12.978 | 41.821 | 1.00 | 7.42 |
| ATOM | 1224 | CG | ASP | 157 | 26.314 | 11.911 | 42.878 | 1.00 | 10.11 |
| ATOM | 1225 | OD1 | ASP | 157 | 26.594 | 10.731 | 42.577 | 1.00 | 9.03 |
| ATOM | 1226 | OD2 | ASP | 157 | 25.822 | 12.253 | 43.982 | 1.00 | 10.69 |
| ATOM | 1227 | C | ASP | 157 | 26.248 | 13.307 | 39.386 | 1.00 | 7.07 |
| ATOM | 1228 | O | ASP | 157 | 25.287 | 12.755 | 38.834 | 1.00 | 8.10 |
| ATOM | 1229 | N | HIS | 158 | 26.661 | 14.502 | 39.062 | 1.00 | 6.19 |
| ATOM | 1230 | CA | HIS | 158 | 26.112 | 15.287 | 38.029 | 1.00 | 7.49 |
| ATOM | 1231 | CB | HIS | 158 | 26.810 | 16.641 | 37.753 | 1.00 | 5.85 |
| ATOM | 1232 | CG | HIS | 158 | 26.559 | 17.707 | 38.769 | 1.00 | 5.01 |
| ATOM | 1233 | ND1 | HIS | 158 | 25.266 | 18.168 | 38.959 | 1.00 | 5.03 |
| ATOM | 1234 | CE1 | HIS | 158 | 25.282 | 19.121 | 39.895 | 1.00 | 4.19 |
| ATOM | 1235 | NE2 | HIS | 158 | 26.557 | 19.288 | 40.292 | 1.00 | 4.26 |
| ATOM | 1236 | CD2 | HIS | 158 | 27.363 | 18.439 | 39.584 | 1.00 | 2.60 |
| ATOM | 1237 | C | HIS | 158 | 26.090 | 14.599 | 36.656 | 1.00 | 6.47 |
| ATOM | 1238 | O | HIS | 158 | 25.171 | 14.898 | 35.913 | 1.00 | 9.43 |
| ATOM | 1239 | N | LEU | 159 | 27.078 | 13.780 | 36.392 | 1.00 | 8.63 |
| ATOM | 1240 | CA | LEU | 159 | 27.220 | 13.074 | 35.122 | 1.00 | 9.33 |
| ATOM | 1241 | CB | LEU | 159 | 28.636 | 12.532 | 34.967 | 1.00 | 7.47 |
| ATOM | 1242 | CG | LEU | 159 | 29.853 | 13.426 | 35.128 | 1.00 | 7.64 |
| ATOM | 1243 | CD1 | LEU | 159 | 31.101 | 12.606 | 34.923 | 1.00 | 6.21 |
| ATOM | 1244 | CD2 | LEU | 159 | 29.832 | 14.677 | 34.295 | 1.00 | 6.97 |
| ATOM | 1245 | C | LEU | 159 | 26.192 | 11.937 | 35.024 | 1.00 | 8.64 |
| ATOM | 1246 | O | LEU | 159 | 25.885 | 11.426 | 33.947 | 1.00 | 7.94 |
| ATOM | 1247 | N | ASN | 160 | 25.605 | 11.583 | 36.164 | 1.00 | 9.77 |
| ATOM | 1248 | CA | AEN | 160 | 24.580 | 10.573 | 36.277 | 1.00 | 9.90 |
| ATOM | 1249 | CB | ASN | 160 | 24.944 | 9.490 | 37.318 | 1.00 | 10.05 |
| ATOM | 1250 | CG | ASN | 160 | 26.211 | 8.761 | 36.969 | 1.00 | 8.81 |
| ATOM | 1251 | OD1 | ASN | 160 | 26.495 | 8.464 | 35.805 | 1.00 | 8.76 |
| ATOM | 1252 | ND2 | ASN | 160 | 27.049 | 8.507 | 37.960 | 1.00 | 11.57 |
| ATOM | 1253 | C | ASN | 160 | 23.207 | 11.100 | 36.542 | 1.00 | 10.13 |
| ATOM | 1254 | O | ASN | 160 | 22.238 | 10.334 | 36.824 | 1.00 | 9.97 |
| ATOM | 1255 | N | GLY | 161 | 22.996 | 12.404 | 36.413 | 1.00 | 10.67 |
| ATOM | 1256 | CA | GLY | 161 | 21.695 | 13.015 | 36.647 | 1.00 | 9.90 |
| ATOM | 1257 | C | GLY | 161 | 21.275 | 13.057 | 38.096 | 1.00 | 10.81 |
| ATOM | 1258 | O | GLY | 161 | 20.049 | 13.064 | 38.394 | 1.00 | 11.80 |
| ATOM | 1259 | N | VAL | 162 | 22.196 | 13.066 | 39.037 | 1.00 | 10.62 |
| ATOM | 1260 | CA | VAL | 162 | 21.975 | 13.082 | 40.469 | 1.00 | 10.24 |
| ATOM | 1261 | CB | VAL | 162 | 22.883 | 11.980 | 41.119 | 1.00 | 12.28 |
| ATOM | 1262 | CG1 | VAL | 162 | 22.819 | 12.009 | 42.654 | 1.00 | 11.36 |
| ATOM | 1263 | CG2 | VAL | 162 | 22.436 | 10.623 | 40.619 | 1.00 | 10.82 |
| ATOM | 1264 | C | VAL | 162 | 22.378 | 14.419 | 41.070 | 1.00 | 9.72 |
| ATOM | 1265 | O | VAL | 162 | 23.480 | 14.919 | 40.817 | 1.00 | 9.92 |
| ATOM | 1266 | N | MET | 163 | 21.507 | 14.997 | 41.900 | 1.00 | 8.97 |
| ATOM | 1267 | CA | MET | 163 | 21.754 | 16.260 | 42.575 | 1.00 | 9.40 |
| ATOM | 1268 | CB | MET | 163 | 20.557 | 17.207 | 42.379 | 1.00 | 9.97 |
| ATOM | 1269 | CG | MET | 163 | 20.282 | 17.521 | 40.923 | 1.00 | 11.50 |
| ATOM | 1270 | SD | MET | 163 | 21.495 | 18.562 | 40.125 | 1.00 | 12.73 |
| ATOM | 1271 | CE | MET | 163 | 21.165 | 20.109 | 40.910 | 1.00 | 11.88 |
| ATOM | 1272 | C | MET | 163 | 22.009 | 15.970 | 44.058 | 1.00 | 10.17 |
| ATOM | 1273 | O | MET | 163 | 21.477 | 14.989 | 44.595 | 1.00 | 10.49 |
| ATOM | 1274 | N | PHE | 164 | 22.853 | 16.760 | 44.715 | 1.00 | 9.65 |
| ATOM | 1275 | CA | PHE | 164 | 23.213 | 16.489 | 46.086 | 1.00 | 8.95 |
| ATOM | 1276 | CB | PHE | 164 | 24.153 | 17.529 | 46.683 | 1.00 | 8.12 |
| ATOM | 1277 | CG | PHE | 164 | 23.519 | 18.850 | 47.009 | 1.00 | 7.63 |
| ATOM | 1278 | CD1 | PHE | 164 | 22.981 | 19.041 | 48.271 | 1.00 | 7.03 |
| ATOM | 1279 | CD2 | PHE | 164 | 23.442 | 19.870 | 46.055 | 1.00 | 6.36 |
| ATOM | 1280 | CE1 | PHE | 164 | 22.357 | 20.248 | 48.597 | 1.00 | 9.27 |
| ATOM | 1281 | CE2 | PHE | 164 | 22.792 | 21.030 | 46.358 | 1.00 | 7.45 |
| ATOM | 1282 | CZ | PHE | 164 | 22.288 | 21.248 | 47.613 | 1.00 | 7.70 |
| ATOM | 1283 | C | PHE | 164 | 22.073 | 16.212 | 47.048 | 1.00 | 8.66 |
| ATOM | 1284 | O | PHE | 164 | 22.221 | 15.427 | 47.999 | 1.00 | 8.17 |
| ATOM | 1285 | N | TYR | 165 | 20.984 | 16.929 | 46.869 | 1.00 | 9.74 |
| ATOM | 1286 | CA | TYR | 165 | 19.833 | 16.874 | 47.754 | 1.00 | 9.53 |
| ATOM | 1287 | CB | TYR | 165 | 18.975 | 18.140 | 47.553 | 1.00 | 10.74 |
| ATOM | 1288 | CG | TYR | 165 | 18.393 | 18.301 | 46.175 | 1.00 | 9.43 |
| ATOM | 1289 | CD1 | TYR | 165 | 17.319 | 17.501 | 45.785 | 1.00 | 9.93 |
| ATOM | 1290 | CE1 | TYR | 165 | 16.744 | 17.616 | 44.529 | 1.00 | 9.33 |
| ATOM | 1291 | CZ | TYR | 165 | 17.210 | 18.604 | 43.679 | 1.00 | 8.93 |
| ATOM | 1292 | OH | TYR | 165 | 16.640 | 18.714 | 42.431 | 1.00 | 11.39 |
| ATOM | 1293 | CE2 | TYR | 165 | 18.278 | 19.393 | 44.033 | 1.00 | 7.81 |
| ATOM | 1294 | CD2 | TYR | 165 | 18.833 | 19.277 | 45.291 | 1.00 | 8.68 |
| ATOM | 1295 | C | TYR | 165 | 19.036 | 15.621 | 47.637 | 1.00 | 11.52 |
| ATOM | 1296 | O | TYR | 165 | 18.088 | 15.381 | 48.431 | 1.00 | 12.08 |
| ATOM | 1297 | N | ASP | 166 | 19.358 | 14.814 | 46.616 | 1.00 | 10.44 |
| ATOM | 1298 | CA | ASP | 166 | 18.686 | 13.533 | 46.441 | 1.00 | 10.72 |
| ATOM | 1299 | CB | ASP | 166 | 19.095 | 12.916 | 45.117 | 1.00 | 11.36 |
| ATOM | 1300 | CG | ASP | 166 | 18.738 | 13.691 | 43.871 | 1.00 | 12.08 |
| ATOM | 1301 | OD1 | ASP | 166 | 17.862 | 14.574 | 43.837 | 1.00 | 13.05 |
| ATOM | 1302 | OD2 | ASP | 166 | 19.284 | 13.344 | 42.800 | 1.00 | 12.90 |
| ATOM | 1303 | C | ASP | 166 | 19.152 | 12.599 | 47.567 | 1.00 | 10.61 |
| ATOM | 1304 | O | ASP | 166 | 18.499 | 11.577 | 47.834 | 1.00 | 9.33 |
| ATOM | 1305 | N | HIS | 167 | 20.291 | 12.921 | 48.205 | 1.00 | 10.36 |
| ATOM | 1306 | CA | HIS | 167 | 20.793 | 12.039 | 49.255 | 1.00 | 11.38 |
| ATOM | 1307 | CB | HIS | 167 | 22.358 | 12.033 | 49.264 | 1.00 | 10.95 |
| ATOM | 1308 | CG | HIS | 167 | 22.964 | 11.631 | 47.957 | 1.00 | 13.93 |
| ATOM | 1309 | ND1 | HIS | 167 | 22.923 | 10.334 | 47.453 | 1.00 | 13.92 |
| ATOM | 1310 | CE1 | HIS | 167 | 23.546 | 10.300 | 46.291 | 1.00 | 13.33 |
| ATOM | 1311 | NE2 | HIS | 167 | 24.004 | 11.501 | 46.018 | 1.00 | 12.92 |
| ATOM | 1312 | CD2 | HIS | 167 | 23.645 | 12.366 | 47.027 | 1.00 | 13.06 |
| ATOM | 1313 | C | HIS | 167 | 20.362 | 12.372 | 50.662 | 1.00 | 12.37 |
| ATOM | 1314 | O | HIS | 167 | 20.858 | 11.734 | 51.630 | 1.00 | 13.63 |
| ATOM | 1315 | N | ILE | 168 | 19.525 | 13.362 | 50.860 | 1.00 | 12.97 |
| ATOM | 1316 | CA | ILE | 168 | 19.109 | 13.854 | 52.159 | 1.00 | 12.73 |
| ATOM | 1317 | CB | ILE | 168 | 18.951 | 15.425 | 52.054 | 1.00 | 13.57 |
| ATOM | 1318 | CG1 | ILE | 168 | 20.228 | 16.110 | 51.593 | 1.00 | 11.58 |
| ATOM | 1319 | CD1 | ILE | 168 | 20.226 | 17.595 | 51.292 | 1.00 | 10.84 |
| ATOM | 1320 | CG2 | ILE | 168 | 18.502 | 15.964 | 53.420 | 1.00 | 12.92 |
| ATOM | 1321 | C | ILE | 168 | 17.838 | 13.216 | 52.672 | 1.00 | 14.26 |
| ATOM | 1322 | O | ILE | 168 | 16.805 | 13.155 | 52.017 | 1.00 | 15.10 |
| ATOM | 1323 | N | ASP | 169 | 17.823 | 12.772 | 53.913 | 1.00 | 15.41 |
| ATOM | 1324 | CA | ASP | 169 | 16.631 | 12.138 | 54.507 | 1.00 | 16.46 |
| ATOM | 1325 | CB | ASP | 169 | 17.078 | 11.500 | 55.840 | 1.00 | 16.03 |
| ATOM | 1326 | CG | ASP | 169 | 15.931 | 10.619 | 56.316 | 1.00 | 17.04 |
| ATOM | 1327 | OD1 | ASP | 169 | 14.943 | 11.119 | 56.861 | 1.00 | 18.29 |
| ATOM | 1328 | OD2 | ASP | 169 | 16.030 | 9.434 | 55.962 | 1.00 | 17.14 |
| ATOM | 1329 | C | ASP | 169 | 15.552 | 13.189 | 54.714 | 1.00 | 17.91 |
| ATOM | 1330 | O | ASP | 169 | 15.803 | 14.268 | 55.270 | 1.00 | 16.68 |
| ATOM | 1331 | N | LYS | 170 | 14.353 | 12.945 | 54.189 | 1.00 | 20.44 |
| ATOM | 1332 | CA | LYS | 170 | 13.289 | 13.920 | 54.322 | 1.00 | 24.90 |
| ATOM | 1333 | CB | LYS | 170 | 11.953 | 13.478 | 53.630 | 1.00 | 28.24 |
| ATOM | 1334 | CG | LYS | 170 | 10.958 | 14.663 | 53.604 | 1.00 | 31.46 |
| ATOM | 1335 | CD | LYS | 170 | 9.539 | 14.285 | 53.173 | 1.00 | 32.94 |
| ATOM | 1336 | CE | LYS | 170 | 8.812 | 13.500 | 54.256 | 1.00 | 33.30 |
| ATOM | 1337 | NZ | LYS | 170 | 7.402 | 13.165 | 53.899 | 1.00 | 34.02 |
| ATOM | 1338 | C | LYS | 170 | 12.943 | 14.155 | 55.804 | 1.00 | 25.11 |
| ATOM | 1339 | O | LYS | 170 | 12.858 | 15.277 | 56.264 | 1.00 | 26.53 |
| ATOM | 1340 | N | ASP | 171 | 12.768 | 13.048 | 56.520 | 1.00 | 24.81 |
| ATOM | 1341 | CA | ASP | 171 | 12.371 | 13.086 | 57.912 | 1.00 | 25.10 |
| ATOM | 1342 | CB | ASP | 171 | 11.725 | 11.776 | 58.342 | 1.00 | 29.27 |
| ATOM | 1343 | CG | ASP | 171 | 10.592 | 11.235 | 57.534 | 1.00 | 31.17 |
| ATOM | 1344 | OD1 | ASP | 171 | 9.713 | 11.934 | 57.004 | 1.00 | 31.40 |
| ATOM | 1345 | OD2 | ASP | 171 | 10.606 | 9.961 | 57.381 | 1.00 | 34.11 |
| ATOM | 1346 | C | ASP | 171 | 13.414 | 13.491 | 58.919 | 1.00 | 23.57 |
| ATOM | 1347 | O | ASP | 171 | 13.096 | 14.249 | 59.861 | 1.00 | 23.55 |
| ATOM | 1348 | N | HIS | 172 | 14.650 | 13.038 | 58.756 | 1.00 | 21.67 |
| ATOM | 1349 | CA | HIS | 172 | 15.718 | 13.379 | 59.697 | 1.00 | 19.37 |
| ATOM | 1350 | CB | HIS | 172 | 16.113 | 12.098 | 60.464 | 1.00 | 21.26 |
| ATOM | 1351 | CG | HIS | 172 | 14.941 | 11.559 | 61.215 | 1.00 | 23.69 |
| ATOM | 1352 | ND1 | HIS | 172 | 14.618 | 12.008 | 62.472 | 1.00 | 24.87 |
| ATOM | 1353 | CE1 | HIS | 172 | 13.515 | 11.392 | 62.882 | 1.00 | 26.42 |
| ATOM | 1354 | NE2 | HIS | 172 | 13.139 | 10.532 | 61.925 | 1.00 | 26.64 |
| ATOM | 1355 | CD2 | HIS | 172 | 14.026 | 10.625 | 60.873 | 1.00 | 25.35 |
| ATOM | 1356 | C | HIS | 172 | 16.950 | 13.850 | 58.932 | 1.00 | 16.97 |
| ATOM | 1357 | O | HIS | 172 | 17.981 | 13.177 | 58.955 | 1.00 | 16.83 |
| ATOM | 1358 | N | PRO | 173 | 16.836 | 14.984 | 58.274 | 1.00 | 14.57 |
| ATOM | 1359 | CA | PRO | 173 | 17.899 | 15.518 | 57.452 | 1.00 | 13.11 |
| ATOM | 1360 | CB | PRO | 173 | 17.279 | 16.758 | 56.843 | 1.00 | 12.46 |
| ATOM | 1361 | CG | PRO | 173 | 16.198 | 17.136 | 57.826 | 1.00 | 12.80 |
| ATOM | 1362 | CD | PRO | 173 | 15.585 | 15.805 | 58.221 | 1.00 | 13.20 |
| ATOM | 1363 | C | PRO | 173 | 19.199 | 15.766 | 58.159 | 1.00 | 13.01 |
| ATOM | 1364 | O | PRO | 173 | 20.244 | 15.711 | 57.538 | 1.00 | 12.71 |
| ATOM | 1365 | N | LEU | 174 | 19.213 | 16.071 | 59.457 | 1.00 | 13.35 |
| ATOM | 1366 | CA | LEU | 174 | 20.415 | 16.341 | 60.183 | 1.00 | 13.25 |
| ATOM | 1367 | CB | LEU | 174 | 20.352 | 17.642 | 60.973 | 1.00 | 14.23 |
| ATOM | 1368 | CG | LEU | 174 | 20.025 | 18.898 | 60.156 | 1.00 | 13.91 |
| ATOM | 1369 | CD1 | LEU | 174 | 20.038 | 20.108 | 61.077 | 1.00 | 16.72 |
| ATOM | 1370 | CD2 | LEU | 174 | 20.967 | 19.113 | 58.996 | 1.00 | 15.55 |
| ATOM | 1371 | C | LEU | 174 | 20.917 | 15.204 | 61.019 | 1.00 | 14.24 |
| ATOM | 1372 | O | LEU | 174 | 21.890 | 15.390 | 61.748 | 1.00 | 14.10 |
| ATOM | 1373 | N | GLN | 175 | 20.363 | 14.016 | 60.855 | 1.00 | 15.52 |
| ATOM | 1374 | CA | GLN | 175 | 20.798 | 12.832 | 61.630 | 1.00 | 17.11 |
| ATOM | 1375 | CB | OLN | 175 | 19.623 | 11.857 | 61.795 | 1.00 | 20.95 |
| ATOM | 1376 | CG | GLN | 175 | 19.941 | 10.655 | 62.677 | 1.00 | 27.18 |
| ATOM | 1377 | CD | GLN | 175 | 18.877 | 9.585 | 62.700 | 1.00 | 31.22 |
| ATOM | 1378 | OE1 | GLN | 175 | 18.540 | 8.994 | 63.751 | 1.00 | 33.20 |
| ATOM | 1379 | NE2 | GLN | 175 | 18.330 | 9.235 | 61.533 | 1.00 | 33.65 |
| ATOM | 1380 | C | GLN | 175 | 21.922 | 12.122 | 60.898 | 1.00 | 16.99 |
| ATOM | 1381 | O | GLN | 175 | 21.713 | 11.588 | 59.798 | 1.00 | 16.15 |
| ATOM | 1382 | N | PRO | 176 | 23.104 | 12.137 | 61.448 | 1.00 | 17.92 |
| ATOM | 1383 | CA | PRO | 176 | 24.264 | 11.514 | 60.860 | 1.00 | 19.07 |
| ATOM | 1384 | CB | PRO | 176 | 25.425 | 11.958 | 61.729 | 1.00 | 18.77 |
| ATOM | 1385 | CG | PRO | 176 | 24.866 | 13.138 | 62.518 | 1.00 | 18.47 |
| ATOM | 1386 | CD | PRO | 176 | 23.452 | 12.771 | 62.748 | 1.00 | 17.86 |
| ATOM | 1387 | C | PRO | 176 | 24.188 | 9.990 | 60.900 | 1.00 | 20.17 |
| ATOM | 1388 | O | PRO | 176 | 23.732 | 9.400 | 61.895 | 1.00 | 20.92 |
| ATOM | 1389 | N | HIS | 177 | 24.619 | 9.352 | 59.807 | 1.00 | 19.53 |
| ATOM | 1390 | CA | HIS | 177 | 24.656 | 7.914 | 59.784 | 1.00 | 20.36 |
| ATOM | 1391 | CB | HIS | 177 | 24.919 | 7.206 | 58.469 | 1.00 | 18.20 |
| ATOM | 1392 | CG | HIS | 177 | 23.802 | 7.337 | 57.474 | 1.00 | 14.95 |
| ATOM | 1393 | ND1 | HIS | 177 | 24.005 | 7.159 | 56.138 | 1.00 | 16.40 |
| ATOM | 1394 | CE1 | HIS | 177 | 22.864 | 7.356 | 55.480 | 1.00 | 16.26 |
| ATOM | 1395 | NE2 | HIS | 177 | 21.958 | 7.707 | 56.368 | 1.00 | 16.00 |
| ATOM | 1396 | CD2 | HIS | 177 | 22.523 | 7.700 | 57.617 | 1.00 | 14.21 |
| ATOM | 1397 | C | HIS | 177 | 25.658 | 7.423 | 60.841 | 1.00 | 21.02 |
| ATOM | 1398 | O | HIS | 177 | 26.693 | 8.019 | 61.082 | 1.00 | 22.02 |
| ATOM | 1399 | N | THR | 178 | 25.255 | 6.325 | 61.480 | 1.00 | 22.22 |
| ATOM | 1400 | CA | THR | 178 | 26.106 | 5.744 | 62.510 | 1.00 | 23.81 |
| ATOM | 1401 | CB | THR | 178 | 25.412 | 4.447 | 63.017 | 1.00 | 26.92 |
| ATOM | 1402 | OG1 | THR | 178 | 24.070 | 4.779 | 63.455 | 1.00 | 28.11 |
| ATOM | 1403 | CG2 | THR | 178 | 26.218 | 3.926 | 64.192 | 1.00 | 27.70 |
| ATOM | 1404 | C | THR | 178 | 27.479 | 5.379 | 61.959 | 1.00 | 23.54 |
| ATOM | 1405 | O | THR | 178 | 27.595 | 4.594 | 61.005 | 1.00 | 25.27 |
| ATOM | 1406 | N | ASP | 179 | 28.540 | 5.880 | 62.536 | 1.00 | 22.85 |
| ATOM | 1407 | CA | ASP | 179 | 29.901 | 5.593 | 62.172 | 1.00 | 22.95 |
| ATOM | 1408 | CB | ASP | 179 | 30.170 | 4.097 | 62.080 | 1.00 | 28.84 |
| ATOM | 1409 | CG | ASP | 179 | 29.950 | 3.241 | 63.312 | 1.00 | 33.14 |
| ATOM | 1410 | OD1 | ASP | 179 | 29.420 | 2.094 | 63.173 | 1.00 | 34.35 |
| ATOM | 1411 | OD2 | ASP | 179 | 30.323 | 3.605 | 64.467 | 1.00 | 34.72 |
| ATOM | 1412 | C | ASP | 179 | 30.399 | 6.357 | 60.953 | 1.00 | 20.84 |
| ATOM | 1413 | O | ASP | 179 | 31.509 | 6.146 | 60.505 | 1.00 | 19.20 |
| ATOM | 1414 | N | ALA | 180 | 29.601 | 7.293 | 60.426 | 1.00 | 19.98 |
| ATOM | 1415 | CA | ALA | 180 | 30.052 | 8.106 | 59.279 | 1.00 | 18.45 |
| ATOM | 1416 | CS | ALA | 180 | 28.857 | 8.752 | 58.604 | 1.00 | 17.76 |
| ATOM | 1417 | C | ALA | 180 | 31.020 | 9.145 | 59.822 | 1.00 | 18.09 |
| ATOM | 1418 | O | ALA | 180 | 30.881 | 9.578 | 60.960 | 1.00 | 18.46 |
| ATOM | 1419 | N | VAL | 181 | 32.063 | 9.501 | 59.124 | 1.00 | 17.49 |
| ATOM | 1420 | CA | VAL | 181 | 33.062 | 10.462 | 59.459 | 1.00 | 17.25 |
| ATOM | 1421 | CB | VAL | 181 | 34.320 | 10.313 | 58.578 | 1.00 | 17.21 |
| ATOM | 1422 | CG1 | VAL | 181 | 35.301 | 11.466 | 58.792 | 1.00 | 17.56 |
| ATOM | 1423 | CG2 | VAL | 181 | 35.050 | 9.012 | 58.805 | 1.00 | 17.06 |
| ATOM | 1424 | C | VAL | 181 | 32.558 | 11.911 | 59.223 | 1.00 | 18.05 |
| ATOM | 1425 | O | VAL | 181 | 32.154 | 12.253 | 58.113 | 1.00 | 16.95 |
| ATOM | 1426 | N | GLU | 182 | 32.586 | 12.714 | 60.268 | 1.00 | 17.72 |
| ATOM | 1427 | CA | GLU | 182 | 32.200 | 14.126 | 60.126 | 1.00 | 18.58 |
| ATOM | 1428 | CB | GLU | 182 | 31.974 | 14.715 | 61.518 | 1.00 | 22.08 |
| ATOM | 1429 | CG | GLU | 182 | 31.570 | 16.170 | 61.478 | 1.00 | 26.15 |
| ATOM | 1430 | CD | GLU | 182 | 31.741 | 16.841 | 62.827 | 1.00 | 31.27 |
| ATOM | 1431 | OE1 | GLU | 182 | 32.208 | 16.207 | 63.810 | 1.00 | 32.77 |
| ATOM | 1432 | OE2 | OLU | 182 | 31.553 | 18.068 | 62.895 | 1.00 | 34.76 |
| ATOM | 1433 | C | GLU | 182 | 33.434 | 14.816 | 59.533 | 1.00 | 18.49 |
| ATOM | 1434 | O | GLU | 182 | 34.509 | 14.839 | 60.154 | 1.00 | 17.55 |
| ATOM | 1435 | N | VAL | 183 | 33.323 | 15.377 | 58.351 | 1.00 | 19.14 |
| ATOM | 1436 | CA | VAL | 183 | 34.454 | 16.036 | 57.698 | 1.00 | 20.41 |
| ATOM | 1437 | CB | VAL | 183 | 34.382 | 15.826 | 56.174 | 1.00 | 18.55 |
| ATOM | 1438 | CG1 | VAL | 183 | 35.474 | 16.547 | 55.414 | 1.00 | 17.81 |
| ATOM | 1439 | CG2 | VAL | 183 | 34.544 | 14.317 | 55.887 | 1.00 | 16.84 |
| ATOM | 1440 | C | VAL | 183 | 34.624 | 17.504 | 58.084 | 1.00 | 23.97 |
| ATOM | 1441 | O | VAL | 183 | 33.925 | 18.419 | 57.618 | 1.00 | 23.28 |
| ATOM | 1442 | N | HIS | 184 | 35.657 | 17.750 | 58.915 | 1.00 | 27.34 |
| ATOM | 1443 | CA | HIS | 184 | 36.010 | 19.078 | 59.381 | 1.00 | 30.88 |
| ATOM | 1444 | CB | HIS | 184 | 36.761 | 19.018 | 60.736 | 1.00 | 31.42 |
| ATOM | 1445 | CG | HIS | 184 | 35.950 | 18.462 | 61.852 | 1.00 | 33.04 |
| ATOM | 1446 | ND1 | HIS | 184 | 36.127 | 17.192 | 62.386 | 1.00 | 33.48 |
| ATOM | 1447 | CE1 | HIS | 184 | 35.239 | 16.983 | 63.324 | 1.00 | 33.63 |
| ATOM | 1448 | NE2 | HIS | 184 | 34.472 | 18.078 | 63.427 | 1.00 | 34.18 |
| ATOM | 1449 | CD2 | HIS | 184 | 34.885 | 19.009 | 62.505 | 1.00 | 33.72 |
| ATOM | 1450 | C | HIS | 184 | 36.944 | 19.774 | 58.398 | 1.00 | 33.03 |
| ATOM | 1451 | OXT | HIS | 184 | 38.145 | 19.370 | 58.369 | 1.00 | 34.40 |
| ATOM | 1452 | O | HIS | 184 | 36.482 | 20.674 | 57.663 | 1.00 | 34.98 |
| ATOM | 1453 | O | WAT | 185 | 28.850 | 21.236 | 41.157 | 1.00 | 7.77 |
| ATOM | 1454 | O | WAT | 186 | 31.430 | 6.491 | 39.145 | 1.00 | 5.36 |
| ATOM | 1455 | O | WAT | 187 | 31.972 | 20.433 | 24.682 | 1.00 | 19.06 |
| ATOM | 1456 | O | WAT | 188 | 43.418 | 17.395 | 32.297 | 1.00 | 7.22 |
| ATOM | 1457 | O | WAT | 189 | 29.795 | 9.718 | 43.035 | 1.00 | 13.01 |
| ATOM | 1458 | O | WAT | 190 | 25.169 | 14.868 | 44.564 | 1.00 | 7.63 |
| ATOM | 1459 | O | WAT | 191 | 42.565 | 13.342 | 49.566 | 1.00 | 12.53 |
| ATOM | 1460 | O | WAT | 192 | 23.545 | 16.936 | 36.967 | 1.00 | 11.47 |
| ATOM | 1461 | O | WAT | 193 | 38.976 | 27.793 | 26.907 | 1.00 | 19.75 |
| ATOM | 1462 | O | WAT | 194 | 35.953 | 1.677 | 55.506 | 1.00 | 8.03 |
| ATOM | 1463 | O | WAT | 195 | 45.203 | 4.786 | 35.591 | 1.00 | 19.10 |
| ATOM | 1464 | O | WAT | 196 | 40.702 | 22.372 | 37.469 | 1.00 | 5.65 |
| ATOM | 1465 | O | WAT | 197 | 18.244 | 27.128 | 33.934 | 1.00 | 9.55 |
| ATOM | 1466 | O | WAT | 198 | 43.230 | 2.407 | 45.385 | 1.00 | 14.44 |
| ATOM | 1467 | O | WAT | 199 | 28.760 | 15.753 | 52.327 | 1.00 | 6.36 |
| ATOM | 1468 | O | WAT | 200 | 39.314 | 16.273 | 54.340 | 1.00 | 10.38 |
| ATOM | 1469 | O | WAT | 201 | 41.628 | 6.864 | 49.085 | 1.00 | 13.25 |
| ATOM | 1470 | O | WAT | 202 | 32.628 | 8.643 | 33.259 | 1.00 | 14.96 |
| ATOM | 1471 | O | WAT | 203 | 23.608 | 15.684 | 31.276 | 1.00 | 11.92 |
| ATOM | 1472 | O | WAT | 204 | 41.619 | 27.241 | 32.862 | 1.00 | 12.62 |
| ATOM | 1473 | O | WAT | 205 | 25.455 | 16.379 | 42.062 | 1.00 | 9.79 |
| ATOM | 1474 | O | WAT | 206 | 23.458 | 25.587 | 46.689 | 1.00 | 9.28 |
| ATOM | 1475 | O | WAT | 207 | 40.092 | 2.673 | 49.703 | 1.00 | 21.98 |
| ATOM | 1476 | O | WAT | 208 | 35.495 | 30.435 | 31.702 | 1.00 | 9.21 |
| ATOM | 1477 | O | WAT | 209 | 40.520 | 4.464 | 47.917 | 1.00 | 17.40 |
| ATOM | 1478 | O | WAT | 210 | 28.201 | 9.930 | 46.793 | 1.00 | 14.09 |
| ATOM | 1479 | O | WAT | 211 | 23.089 | 19.943 | 52.306 | 1.00 | 14.82 |
| ATOM | 1480 | O | WAT | 212 | 44.399 | 21.289 | 35.915 | 1.00 | 13.38 |
| ATOM | 1481 | O | WAT | 213 | 38.302 | 11.682 | 56.310 | 1.00 | 13.86 |
| ATOM | 1482 | O | WAT | 214 | 29.194 | 31.320 | 26.877 | 1.00 | 14.92 |
| ATOM | 1483 | O | WAT | 215 | 41.034 | 0.350 | 43.151 | 1.00 | 8.79 |
| ATOM | 1484 | O | WAT | 216 | 37.315 | 5.552 | 55.980 | 1.00 | 15.69 |
| ATOM | 1485 | O | WAT | 217 | 28.681 | 5.735 | 37.072 | 1.00 | 27.33 |
| ATOM | 1486 | O | WAT | 218 | 27.519 | 26.142 | 27.532 | 1.00 | 20.83 |
| ATOM | 1487 | O | WAT | 220 | 26.523 | 16.871 | 54.153 | 1.00 | 7.21 |
| ATOM | 1488 | O | WAT | 221 | 26.631 | 8.829 | 40.556 | 1.00 | 13.85 |
| ATOM | 1489 | O | WAT | 222 | 42.195 | 14.491 | 24.181 | 1.00 | 18.25 |
| ATOM | 1490 | O | WAT | 223 | 39.484 | 2.299 | 47.684 | 1.00 | 17.37 |
| ATOM | 1491 | O | WAT | 224 | 42.696 | 5.852 | 33.808 | 1.00 | 21.32 |
| ATOM | 1492 | O | WAT | 225 | 21.738 | 21.298 | 24.368 | 1.00 | 34.50 |
| ATOM | 1493 | O | WAT | 226 | 22.987 | 10.820 | 52.989 | 1.00 | 14.83 |
| ATOM | 1494 | O | WAT | 227 | 46.793 | 19.037 | 41.919 | 1.00 | 17.41 |
| ATOM | 1495 | O | WAT | 228 | 50.134 | 12.914 | 25.200 | 1.00 | 13.49 |
| ATOM | 1496 | O | WAT | 229 | 34.941 | 32.358 | 33.918 | 1.00 | 10.04 |
| ATOM | 1497 | O | WAT | 230 | 29.840 | 4.973 | 57.907 | 1.00 | 13.19 |
| ATOM | 1498 | O | WAT | 231 | 41.476 | 32.535 | 41.932 | 1.00 | 16.82 |
| ATOM | 1499 | O | WAT | 233 | 47.577 | 10.560 | 24.957 | 1.00 | 14.85 |
| ATOM | 1500 | O | WAT | 234 | 31.423 | 7.923 | 35.851 | 1.00 | 18.25 |
| ATOM | 1501 | O | WAT | 235 | 24.429 | 27.131 | 29.298 | 1.00 | 20.86 |
| ATOM | 1502 | O | WAT | 236 | 45.316 | 2.958 | 43.842 | 1.00 | 16.57 |
| ATOM | 1503 | O | WAT | 237 | 46.300 | 14.369 | 48.285 | 1.00 | 15.62 |
| ATOM | 1504 | O | WAT | 238 | 22.551 | 21.212 | 56.437 | 1.00 | 20.75 |
| ATOM | 1505 | O | WAT | 240 | 42.496 | 7.817 | 57.030 | 1.00 | 36.21 |
| ATOM | 1506 | O | WAT | 241 | 29.753 | 7.840 | 45.086 | 1.00 | 20.48 |
| ATOM | 1507 | O | WAT | 242 | 44.157 | 18.020 | 35.284 | 1.00 | 15.10 |
| ATOM | 1508 | O | WAT | 243 | 32.571 | 28.209 | 41.063 | 1.00 | 26.82 |
| ATOM | 1509 | O | WAT | 244 | 29.928 | 3.636 | 51.745 | 1.00 | 17.99 |
| ATOM | 1510 | O | WAT | 245 | 17.606 | 26.422 | 41.913 | 1.00 | 14.25 |
| ATOM | 1511 | O | WAT | 246 | 20.589 | 12.991 | 55.488 | 1.00 | 19.81 |
| ATOM | 1512 | O | WAT | 247 | 39.341 | 29.548 | 29.158 | 1.00 | 18.35 |
| ATOM | 1513 | O | WAT | 248 | 17.156 | 16.295 | 61.445 | 1.00 | 12.69 |
| ATOM | 1514 | O | WAT | 249 | 24.354 | 14.243 | 33.242 | 1.00 | 12.22 |
| ATOM | 1515 | O | WAT | 250 | 32.830 | 14.766 | 20.087 | 1.00 | 25.25 |
| ATOM | 1516 | O | WAT | 251 | 16.815 | 19.536 | 36.675 | 1.00 | 15.62 |
| ATOM | 1517 | O | WAT | 252 | 28.441 | 34.415 | 30.350 | 1.00 | 14.51 |
| ATOM | 1518 | O | WAT | 253 | 26.031 | 8.747 | 44.300 | 1.00 | 20.25 |
| ATOM | 1519 | O | WAT | 254 | 43.077 | 16.183 | 18.629 | 1.00 | 31.67 |
| ATOM | 1520 | O | WAT | 256 | 33.197 | 19.064 | 19.853 | 1.00 | 24.22 |
| ATOM | 1521 | O | WAT | 257 | 30.278 | 20.250 | 16.867 | 1.00 | 42.28 |
| ATOM | 1522 | O | WAT | 258 | 20.798 | 17.479 | 26.186 | 1.00 | 29.53 |
| ATOM | 1523 | O | WAT | 259 | 35.416 | 13.350 | 21.607 | 1.00 | 22.56 |
| ATOM | 1524 | O | WAT | 260 | 35.637 | 44.109 | 41.691 | 1.00 | 21.90 |
| ATOM | 1525 | O | WAT | 261 | 24.100 | 18.625 | 43.187 | 1.00 | 8.00 |
| ATOM | 1526 | O | WAT | 263 | 22.843 | 12.530 | 27.491 | 1.00 | 22.94 |
| ATOM | 1527 | O | WAT | 264 | 44.179 | 24.395 | 43.693 | 1.00 | 23.48 |
| ATOM | 1528 | O | WAT | 265 | 18.860 | 10.472 | 42.233 | 1.00 | 22.82 |
| ATOM | 1529 | O | WAT | 266 | 16.026 | 26.688 | 39.851 | 1.00 | 14.46 |
| ATOM | 1530 | O | WAT | 267 | 26.345 | 14.750 | 24.426 | 1.00 | 23.54 |
| ATOM | 1531 | O | WAT | 268 | 37.901 | 14.303 | 60.873 | 1.00 | 57.94 |
| ATOM | 1532 | O | WAT | 269 | 23.911 | 8.885 | 51.213 | 1.00 | 19.17 |
| ATOM | 1533 | O | WAT | 270 | 25.088 | 18.293 | 25.334 | 1.00 | 21.16 |
| ATOM | 1534 | O | WAT | 271 | 32.345 | 12.423 | 22.061 | 1.00 | 26.85 |
| ATOM | 1535 | O | WAT | 272 | 29.849 | 8.405 | 23.646 | 1.00 | 22.55 |
| ATOM | 1536 | O | WAT | 273 | 38.933 | 19.380 | 16.508 | 1.00 | 29.99 |
| ATOM | 1537 | O | WAT | 274 | 28.930 | 6.191 | 30.272 | 1.00 | 30.24 |
| ATOM | 1538 | O | WAT | 277 | 51.699 | 18.896 | 51.858 | 1.00 | 26.44 |
| ATOM | 1539 | O | WAT | 278 | 26.732 | 24.016 | 51.882 | 1.00 | 20.15 |
| ATOM | 1540 | O | WAT | 280 | 35.901 | 30.991 | 27.045 | 1.00 | 31.15 |
| ATOM | 1541 | O | WAT | 281 | 34.878 | 34.657 | 45.573 | 1.00 | 23.59 |
| ATOM | 1542 | O | WAT | 282 | 22.582 | 24.196 | 49.931 | 1.00 | 17.52 |
| ATOM | 1543 | O | WAT | 283 | 25.665 | 18.480 | 61.477 | 1.00 | 22.72 |
| ATOM | 1544 | O | WAT | 285 | 16.617 | 23.741 | 53.679 | 1.00 | 22.92 |
| ATOM | 1545 | O | WAT | 286 | 27.425 | 5.238 | 57.616 | 1.00 | 28.57 |
| ATOM | 1546 | O | WAT | 287 | 24.194 | 17.156 | 62.543 | 1.00 | 22.92 |
| ATOM | 1547 | O | WAT | 288 | 43.605 | 13.871 | 55.205 | 1.00 | 47.34 |
| ATOM | 1548 | O | WAT | 289 | 14.252 | 10.820 | 52.850 | 1.00 | 28.43 |
| ATOM | 1549 | O | WAT | 291 | 29.445 | 0.878 | 44.981 | 1.00 | 19.69 |
| ATOM | 1550 | O | WAT | 292 | 33.986 | 13.974 | 64.333 | 1.00 | 37.04 |
| ATOM | 1551 | O | WAT | 293 | 41.717 | 34.544 | 35.754 | 1.00 | 26.74 |
| ATOM | 1552 | O | WAT | 294 | 44.822 | 15.305 | 22.321 | 1.00 | 16.03 |
| ATOM | 1553 | O | WAT | 295 | 36.295 | 31.861 | 47.655 | 1.00 | 26.65 |
| ATOM | 1554 | O | WAT | 296 | 42.599 | 28.766 | 43.606 | 1.00 | 26.10 |
| ATOM | 1555 | O | WAT | 297 | 39.805 | 5.761 | 18.396 | 1.00 | 41.39 |
| ATOM | 1556 | O | WAT | 298 | 13.973 | 17.827 | 35.310 | 1.00 | 54.73 |
| ATOM | 1557 | O | WAT | 299 | 27.996 | 7.316 | 26.790 | 1.00 | 18.08 |
| ATOM | 1558 | O | WAT | 300 | 42.886 | 21.511 | 50.516 | 1.00 | 18.76 |
| ATOM | 1559 | O | WAT | 303 | 49.197 | 25.130 | 46.385 | 1.00 | 34.63 |
| ATOM | 1560 | O | WAT | 304 | 30.514 | 20.096 | 22.934 | 1.00 | 26.25 |
| ATOM | 1561 | O | WAT | 305 | 16.070 | 11.629 | 32.114 | 1.00 | 33.09 |
| ATOM | 1562 | O | WAT | 307 | 45.009 | 9.505 | 49.911 | 1.00 | 38.13 |
| ATOM | 1563 | O | WAT | 308 | 19.596 | 10.468 | 29.457 | 1.00 | 17.29 |
| ATOM | 1564 | O | WAT | 309 | 15.205 | 14.450 | 44.995 | 1.00 | 14.06 |
| ATOM | 1565 | O | WAT | 310 | 29.356 | 16.954 | 20.232 | 1.00 | 31.55 |
| ATOM | 1566 | O | WAT | 311 | 24.108 | 38.264 | 35.844 | 1.00 | 39.11 |
| ATOM | 1567 | O | WAT | 312 | 35.407 | 6.361 | 25.290 | 1.00 | 30.83 |
| ATOM | 1568 | O | WAT | 313 | 39.712 | 31.481 | 26.164 | 1.00 | 59.55 |
| ATOM | 1569 | O | WAT | 314 | 46.008 | 26.765 | 46.643 | 1.00 | 24.46 |
| ATOM | 1570 | O | WAT | 315 | 31.206 | 32.756 | 27.472 | 1.00 | 25.26 |
| ATOM | 1571 | O | WAT | 316 | 50.563 | 22.574 | 47.365 | 1.00 | 33.15 |
| ATOM | 1572 | O | WAT | 317 | 44.191 | 6.963 | 54.611 | 1.00 | 37.60 |
| ATOM | 1573 | O | WAT | 318 | 25.966 | 3.136 | 59.271 | 1.00 | 35.51 |
| ATOM | 1574 | O | WAT | 319 | 37.521 | 28.687 | 19.179 | 1.00 | 42.82 |
| ATOM | 1575 | O | WAT | 320 | 37.860 | 6.732 | 23.019 | 1.00 | 29.39 |
| ATOM | 1576 | O | WAT | 321 | 16.404 | 30.148 | 38.616 | 1.00 | 44.90 |
| ATOM | 1577 | O | WAT | 322 | 20.948 | 10.666 | 55.483 | 1.00 | 20.70 |
| ATOM | 1578 | O | WAT | 323 | 42.010 | 40.095 | 40.065 | 1.00 | 30.74 |
| ATOM | 1579 | O | WAT | 324 | 34.974 | 43.535 | 31.498 | 1.00 | 33.73 |
| ATOM | 1580 | O | WAT | 325 | 36.339 | 21.393 | 17.438 | 1.00 | 22.90 |
| ATOM | 1581 | O | WAT | 326 | 22.573 | 26.604 | 27.035 | 1.00 | 25.45 |
| ATOM | 1582 | O | WAT | 327 | 43.925 | 1.275 | 33.700 | 1.00 | 31.52 |
| ATOM | 1583 | O | WAT | 328 | 44.055 | 25.497 | 31.799 | 1.00 | 25.75 |
| ATOM | 1584 | O | WAT | 329 | 21.385 | 6.303 | 61.996 | 1.00 | 33.54 |
| ATOM | 1585 | O | WAT | 330 | 29.459 | 11.771 | 63.481 | 1.00 | 30.61 |
| ATOM | 1586 | O | WAT | 331 | 44.458 | 29.701 | 35.168 | 1.00 | 21.73 |
| ATOM | 1587 | O | WAT | 332 | 44.874 | 3.401 | 28.884 | 1.00 | 21.60 |
| ATOM | 1588 | O | WAT | 333 | 35.791 | 46.126 | 38.798 | 1.00 | 31.81 |
| ATOM | 1589 | O | WAT | 335 | 42.315 | 35.196 | 45.915 | 1.00 | 81.46 |
| ATOM | 1590 | O | WAT | 336 | 31.457 | 36.447 | 45.501 | 1.00 | 48.90 |
| ATOM | 1591 | O | WAT | 337 | 21.963 | 8.289 | 49.003 | 1.00 | 25.78 |
| ATOM | 1592 | O | WAT | 338 | 46.389 | 28.795 | 37.209 | 1.00 | 25.28 |
| ATOM | 1593 | O | WAT | 339 | 24.612 | 30.116 | 24.354 | 1.00 | 52.56 |
| ATOM | 1594 | O | WAT | 340 | 32.083 | 2.650 | 30.885 | 1.00 | 67.46 |
| ATOM | 1595 | O | WAT | 341 | 44.830 | 28.999 | 40.931 | 1.00 | 47.88 |
| ATOM | 1596 | O | WAT | 342 | 30.337 | 12.782 | 23.955 | 1.00 | 18.21 |
| ATOM | 1597 | O | WAT | 343 | 28.938 | 3.896 | 35.075 | 1.00 | 33.18 |
| ATOM | 1598 | O | WAT | 344 | 14.617 | 18.243 | 39.459 | 1.00 | 29.61 |
| ATOM | 1599 | O | WAT | 345 | 24.634 | 22.916 | 24.622 | 1.00 | 27.73 |
| ATOM | 1600 | O | WAT | 346 | 39.434 | 34.582 | 32.483 | 1.00 | 20.28 |
| ATOM | 1601 | O | WAT | 347 | 12.161 | 23.144 | 40.518 | 1.00 | 61.26 |
| ATOM | 1602 | O | WAT | 348 | 27.481 | 2.007 | 50.279 | 1.00 | 79.56 |
| ATOM | 1603 | O | WAT | 349 | 42.979 | 34.755 | 39.897 | 1.00 | 44.74 |
| ATOM | 1604 | O | WAT | 350 | 28.778 | 41.078 | 42.112 | 1.00 | 28.20 |
| ATOM | 1605 | O | WAT | 351 | 17.300 | 10.548 | 37.874 | 1.00 | 43.80 |
| ATOM | 1606 | O | WAT | 352 | 19.099 | 8.296 | 59.238 | 1.00 | 40.38 |
| ATOM | 1607 | O | WAT | 353 | 27.220 | 31.276 | 48.083 | 1.00 | 40.23 |
| ATOM | 1608 | O | WAT | 354 | 25.981 | 34.775 | 41.644 | 1.00 | 40.74 |
| ATOM | 1609 | O | WAT | 355 | 27.143 | 6.951 | 48.473 | 1.00 | 25.01 |
| ATOM | 1610 | O | WAT | 356 | 38.151 | 11.528 | 18.954 | 1.00 | 25.07 |
| ATOM | 1611 | O | WAT | 357 | 19.762 | 7.319 | 56.021 | 1.00 | 25.55 |
| ATOM | 1612 | O | WAT | 358 | 28.144 | 1.582 | 55.375 | 1.00 | 89.67 |
| ATOM | 1613 | O | WAT | 360 | 27.638 | 16.644 | 62.799 | 1.00 | 23.18 |
| ATOM | 1614 | O | WAT | 361 | 14.684 | 31.678 | 44.174 | 1.00 | 71.66 |
| ATOM | 1615 | O | WAT | 362 | 15.650 | 11.558 | 66.663 | 1.00 | 64.23 |
| ATOM | 1616 | O | WAT | 363 | 15.237 | 18.359 | 54.178 | 1.00 | 39.41 |
| ATOM | 1617 | O | WAT | 364 | 43.172 | 16.847 | 51.474 | 1.00 | 22.97 |
| ATOM | 1618 | O | WAT | 365 | 18.986 | 26.729 | 27.844 | 1.00 | 48.99 |
| ATOM | 1619 | O | WAT | 366 | 27.139 | 19.402 | 23.399 | 1.00 | 29.96 |
| ATOM | 1620 | O | WAT | 367 | 45.600 | 0.114 | 38.791 | 1.00 | 74.35 |
| ATOM | 1621 | O | WAT | 369 | 19.987 | 11.960 | 57.958 | 1.00 | 16.62 |
| ATOM | 1622 | O | WAT | 372 | 27.335 | 27.231 | 54.352 | 1.00 | 40.25 |
| ATOM | 1623 | O | WAT | 373 | 15.884 | 15.171 | 31.990 | 1.00 | 16.33 |
| ATOM | 1624 | O | WAT | 374 | 15.330 | 24.772 | 50.039 | 1.00 | 32.03 |
| ATOM | 1625 | O | WAT | 377 | 43.724 | 3.843 | 52.380 | 1.00 | 65.10 |
| ATOM | 1626 | O | WAT | 378 | 38.825 | 38.327 | 52.464 | 1.00 | 49.54 |
| ATOM | 1627 | O | WAT | 379 | 46.669 | 23.629 | 33.133 | 1.00 | 28.01 |
| ATOM | 1628 | O | WAT | 380 | 25.700 | 12.197 | 66.584 | 1.00 | 89.69 |
| ATOM | 1629 | O | WAT | 381 | 16.946 | 9.817 | 50.702 | 1.00 | 39.01 |
| ATOM | 1630 | O | WAT | 382 | 28.300 | 9.517 | 63.491 | 1.00 | 33.60 |
| ATOM | 1631 | O | WAT | 384 | 46.564 | 10.837 | 53.832 | 1.00 | 46.92 |
| ATOM | 1632 | O | WAT | 385 | 19.884 | 8.624 | 51.494 | 1.00 | 27.80 |
| ATOM | 1633 | O | WAT | 387 | 40.902 | 2.984 | 29.363 | 1.00 | 31.27 |
| ATOM | 1634 | O | WAT | 391 | 34.316 | 2.463 | 62.378 | 1.00 | 58.47 |
| ATOM | 1635 | O | WAT | 392 | 33.435 | 11.543 | 52.204 | 1.00 | 30.98 |
| ATOM | 1636 | O | WAT | 393 | 31.770 | 25.813 | 26.248 | 1.00 | 27.06 |
| ATOM | 1637 | O | WAT | 400 | 29.567 | 21.566 | 48.768 | 1.00 | 15.75 |
| ATOM | 1638 | O | WAT | 402 | 48.664 | 8.376 | 47.162 | 1.00 | 18.07 |
| ATOM | 1639 | O | WAT | 403 | 27.502 | 36.401 | 36.418 | 1.00 | 21.54 |
| ATOM | 1640 | O | WAT | 404 | 38.545 | 3.037 | 55.400 | 1.00 | 13.22 |
| ATOM | 1641 | O | WAT | 405 | 31.109 | 23.890 | 44.564 | 1.00 | 12.95 |
| ATOM | 1642 | O | WAT | 406 | 37.990 | 6.814 | 28.956 | 1.00 | 20.51 |
| ATOM | 1643 | O | WAT | 407 | 23.258 | 29.488 | 41.877 | 1.00 | 11.41 |
| ATOM | 1644 | O | WAT | 409 | 38.433 | 6.626 | 31.944 | 1.00 | 19.15 |
| ATOM | 1645 | O | WAT | 410 | 31.295 | 21.118 | 43.998 | 1.00 | 14.11 |
| ATOM | 1646 | O | WAT | 411 | 29.526 | 7.658 | 41.193 | 1.00 | 14.42 |
| ATOM | 1647 | O | WAT | 412 | 47.253 | 15.532 | 34.182 | 1.00 | 15.59 |
| ATOM | 1648 | O | WAT | 413 | 32.025 | 22.355 | 49.225 | 1.00 | 22.54 |
| ATOM | 1649 | O | WAT | 414 | 35.003 | 0.189 | 57.857 | 1.00 | 17.18 |
| ATOM | 1650 | O | WAT | 415 | 38.803 | 38.578 | 33.800 | 1.00 | 50.11 |
| ATOM | 1651 | O | WAT | 416 | 31.617 | 3.615 | 39.556 | 1.00 | 15.08 |
| ATOM | 1652 | O | WAT | 417 | 41.233 | 19.520 | 51.473 | 1.00 | 26.94 |
| ATOM | 1653 | O | WAT | 418 | 34.336 | 27.496 | 25.242 | 1.00 | 32.64 |
| ATOM | 1654 | O | WAT | 419 | 31.486 | 2.145 | 58.952 | 1.00 | 26.96 |
| ATOM | 1655 | O | WAT | 420 | 20.483 | 8.464 | 45.003 | 1.00 | 39.84 |
| ATOM | 1656 | O | WAT | 421 | 38.402 | 6.719 | 60.710 | 1.00 | 53.78 |
| ATOM | 1657 | O | WAT | 422 | 17.225 | 14.988 | 64.662 | 1.00 | 27.63 |
| ATOM | 1658 | O | WAT | 423 | 29.858 | 4.786 | 43.647 | 1.00 | 25.15 |
| ATOM | 1659 | O | WAT | 424 | 30.773 | 22.384 | 54.282 | 1.00 | 36.55 |
| ATOM | 1660 | O | WAT | 425 | 38.526 | 14.414 | 56.721 | 1.00 | 20.85 |
| ATOM | 1661 | O | WAT | 426 | 30.456 | 20.512 | 46.952 | 1.00 | 19.94 |
| ATOM | 1662 | O | WAT | 427 | 22.093 | 34.199 | 38.603 | 1.00 | 30.42 |
| ATOM | 1663 | O | WAT | 428 | 29.003 | 23.510 | 42.193 | 1.00 | 14.64 |
| ATOM | 1664 | O | WAT | 430 | 24.931 | 30.471 | 52.337 | 1.00 | 30.16 |
| ATOM | 1665 | O | WAT | 431 | 48.705 | 28.493 | 42.035 | 1.00 | 55.68 |
| ATOM | 1666 | O | WAT | 432 | 39.117 | 4.219 | 32.847 | 1.00 | 25.71 |
| ATOM | 1667 | O | WAT | 433 | 26.609 | 27.683 | 58.327 | 1.00 | 27.50 |
| ATOM | 1668 | O | WAT | 434 | 39.186 | −0.478 | 36.174 | 1.00 | 49.55 |
| ATOM | 1669 | O | WAT | 435 | 41.271 | 4.828 | 21.864 | 1.00 | 39.72 |
| ATOM | 1670 | O | WAT | 436 | 41.092 | 33.182 | 29.047 | 1.00 | 32.94 |
| ATOM | 1671 | O | WAT | 437 | 15.296 | 21.832 | 45.786 | 1.00 | 40.79 |
| ATOM | 1672 | O | WAT | 438 | 28.338 | 42.926 | 45.181 | 1.00 | 54.46 |
| ATOM | 1673 | O | WAT | 439 | 18.910 | 32.039 | 47.096 | 1.00 | 34.92 |
| ATOM | 1674 | O | WAT | 440 | 20.737 | 30.240 | 33.890 | 1.00 | 27.30 |
| ATOM | 1675 | O | WAT | 441 | 39.566 | 2.311 | 52.603 | 1.00 | 9.84 |
| ATOM | 1676 | O | WAT | 442 | 26.307 | 7.449 | 29.241 | 1.00 | 24.87 |
| ATOM | 1677 | O | WAT | 443 | 33.345 | 6.388 | 32.193 | 1.00 | 25.75 |
| ATOM | 1678 | O | WAT | 444 | 31.294 | 5.444 | 34.030 | 1.00 | 30.09 |
| ATOM | 1679 | O | WAT | 445 | 28.477 | 5.556 | 33.091 | 1.00 | 33.90 |
| ATOM | 1680 | O | WAT | 446 | 35.818 | 5.354 | 27.499 | 1.00 | 34.01 |
| ATOM | 1681 | O | WAT | 447 | 38.643 | 6.449 | 25.935 | 1.00 | 36.56 |
| ATOM | 1682 | O | WAT | 448 | 32.925 | 5.503 | 23.694 | 1.00 | 34.87 |
| ATOM | 1683 | O | WAT | 449 | 36.725 | 28.997 25.274 | 1.00 | 30.86 | |
| ATOM | 1684 | O | WAT | 450 | 33.363 | 29.455 | 26.675 | 1.00 | 37.40 |
| ATOM | 1685 | O | WAT | 451 | 41.451 | 27.420 | 25.703 | 1.00 | 33.62 |
| ATOM | 1686 | O | WAT | 452 | 29.410 | 26.542 | 26.305 | 1.00 | 29.99 |
| ATOM | 1687 | O | WAT | 453 | 36.755 | 35.395 | 27.932 | 1.00 | 31.87 |
| ATOM | 1688 | O | WAT | 454 | 38.358 | 33.650 | 29.807 | 1.00 | 27.03 |
| ATOM | 1689 | O | WAT | 455 | 37.465 | 31.045 | 29.786 | 1.00 | 24.00 |
| ATOM | 1690 | O | WAT | 456 | 36.203 | 38.857 | 29.055 | 1.00 | 33.74 |
| ATOM | 1691 | O | WAT | 457 | 43.139 | 26.818 | 23.216 | 1.00 | 42.86 |
| ATOM | 1692 | O | WAT | 458 | 44.010 | 27.925 | 26.702 | 1.00 | 35.47 |
| ATOM | 1693 | O | WAT | 459 | 42.654 | 24.489 | 23.829 | 1.00 | 36.86 |
| ATOM | 1694 | O | WAT | 460 | 41.901 | 30.150 | 29.907 | 1.00 | 28.79 |
| ATOM | 1695 | O | WAT | 461 | 26.772 | 28.402 | 27.845 | 1.00 | 24.54 |
| ATOM | 1696 | O | WAT | 462 | 26.549 | 25.016 | 24.992 | 1.00 | 30.41 |
| ATOM | 1697 | O | WAT | 463 | 24.198 | 25.675 | 25.291 | 1.00 | 29.34 |
| ATOM | 1698 | O | WAT | 464 | 18.389 | 23.209 | 26.099 | 1.00 | 27.72 |
| ATOM | 1699 | O | WAT | 465 | 15.792 | 17.751 | 27.506 | 1.00 | 35.38 |
| ATOM | 1700 | O | WAT | 466 | 18.177 | 19.068 | 27.187 | 1.00 | 29.61 |
| ATOM | 1701 | O | WAT | 467 | 20.277 | 34.110 | 40.402 | 1.00 | 39.33 |
| ATOM | 1702 | O | WAT | 468 | 22.420 | 34.685 | 35.945 | 1.00 | 32.47 |
| ATOM | 1703 | O | WAT | 469 | 25.586 | 36.936 | 38.120 | 1.00 | 30.14 |
| ATOM | 1704 | O | WAT | 470 | 22.975 | 32.962 | 41.300 | 1.00 | 37.30 |
| ATOM | 1705 | O | WAT | 471 | 22.668 | 19.271 | 25.898 | 1.00 | 29.84 |
| ATOM | 1706 | O | WAT | 472 | 18.891 | 9.006 | 33.972 | 1.00 | 36.50 |
| ATOM | 1707 | O | WAT | 473 | 24.180 | 8.334 | 27.833 | 1.00 | 29.82 |
| ATOM | 1708 | O | WAT | 474 | 26.583 | 4.932 | 30.103 | 1.00 | 31.47 |
| ATOM | 1709 | O | WAT | 475 | 35.276 | 12.918 | 19.062 | 1.00 | 29.07 |
| ATOM | 1710 | O | WAT | 476 | 37.941 | 14.051 | 18.038 | 1.00 | 30.43 |
| ATOM | 1711 | O | WAT | 477 | 38.446 | 16.323 | 16.217 | 1.00 | 33.99 |
| ATOM | 1712 | O | WAT | 478 | 38.163 | 8.369 | 18.377 | 1.00 | 28.75 |
| ATOM | 1713 | O | WAT | 479 | 42.854 | 7.090 | 21.196 | 1.00 | 35.78 |
| ATOM | 1714 | O | WAT | 480 | 43.719 | 8.314 | 25.189 | 1.00 | 18.37 |
| ATOM | 1715 | O | WAT | 481 | 44.810 | 8.599 | 19.951 | 1.00 | 33.64 |
| ATOM | 1716 | O | WAT | 482 | 47.966 | 7.671 | 21.852 | 1.00 | 31.71 |
| ATOM | 1717 | O | WAT | 483 | 45.820 | 13.136 | 19.737 | 1.00 | 32.03 |
| ATOM | 1718 | O | WAT | 484 | 31.751 | 17.094 | 18.635 | 1.00 | 30.24 |
| ATOM | 1719 | O | WAT | 485 | 27.993 | 14.973 | 20.979 | 1.00 | 33.51 |
| ATOM | 1720 | O | WAT | 486 | 26.220 | 11.398 | 22.499 | 1.00 | 33.95 |
| ATOM | 1721 | O | WAT | 487 | 28.510 | 14.814 | 17.996 | 1.00 | 35.70 |
| ATOM | 1722 | O | WAT | 488 | 33.549 | 20.609 | 17.456 | 1.00 | 30.90 |
| ATOM | 1723 | O | WAT | 489 | 27.960 | 13.392 | 23.087 | 1.00 | 26.06 |
| ATOM | 1724 | O | WAT | 490 | 40.175 | 20.917 | 14.980 | 1.00 | 37.89 |
| ATOM | 1725 | ZN | Zn | 500 | 26.949 | 20.605 | 41.894 | 1.00 | 12.48 |
| END | | | | | | | | | |

What is claimed is:

1. A molecule or molecular complex comprising at least a portion of an S. aureus peptide deformylase or an S. aureus peptide deformylase-like active site comprising amino acids Arg56, Ser57, Gly58, Val59, Gly60, Leu61, Ala62, Leu105, Thr107, Gly108, Glu109, Gly110, Cys111, Leu112, Ser113, Ile150, Val151, His154, Glu155, and His158, the active site being defined by a set of points having a root mean square deviation of less than about 0.35 Å from points representing the backbone atoms of said amino acids as represented by structure coordinates listed in Table 1.

2. The molecule or molecular complex of claim 1 further comprising a coordinated metal ion selected from the group of metals consisting of Fe, Zn, Ni and combinations thereof.

3. The molecule or molecular complex of claim 2, wherein the metal ion is coordinated by the amino acids Cys111, His154, and His158.

4. A molecule or molecular complex comprising at least a portion of an S. aureus peptide deformylase or an S. aureus peptide deformylase-like active site comprising amino acids Arg56, Ser57, Gly58, Val59, Gly60, Leu61, Ala62, Leu105, Thr107, Gly108, Glu109, Gly110, Cys111, Leu112, Ser113, Ile150, Val151, His154, Glu155, and His158, the active site being defined by a set of points having a root mean square deviation of less than about 0.8 Å from points representing the backbone atoms of said amino acids as represented by structure coordinates listed in Table 1.

5. The molecule or molecular complex of claim 4 further comprising a coordinated metal ion selected from the group of metals consisting of Fe, Zn, Ni and combinations thereof.

6. The molecule or molecular complex of claim 5, wherein the metal ion is coordinated by the amino acids Cys111, His154, and His158.

7. A machine-readable data storage medium comprising a data storage material encoded with machine readable data which, when using a machine programmed with instructions for using said data, displays a graphical three-dimensional representation of at least one molecule or molecular complex selected from the group consisting of:
(i) a molecule or molecular complex comprising at least a portion of an S. aureus peptide deformylase or an S. aureus peptide deformylase-like active site comprising amino acids Arg56, Ser57, Gly58, Val59, Gly60, Leu61, Ala62, Leu105, Thr107, Gly108, Glu109, Gly110, Cys111, Leu112, Ser113, Ile150, Val151, His154, Glu155, and His158, the active site being defined by a set of points having a root mean square deviation of less than about 0.35 Å from points representing the backbone atoms of said amino acids as represented by structure coordinates listed in Table 1; and
(iii) a molecule or molecular complex that is structurally homologous to an S. aureus peptide deformylase molecule or molecular complex, wherein the S. aureus peptide deformylase molecule or molecular complex is represented by structure coordinates listed in Table 1.

8. A computer-assisted method for identifying a potential modifier of S. aureus peptide deformylase activity comprising:
supplying a computer modeling application with a set of structure coordinates of a molecule or molecular complex, the molecule or molecular complex comprising at least a portion of at least one S. aureus peptide deformylase or S. aureus peptide deformylase-like active site, the active site comprising amino acids Arg56, Ser57, Gly58, Val59, Gly60, Leu61, Ala62, Leu105, Thr107, Gly108, Glu109, Gly110, Cys111, Leu112, Ser113, Ile150, Val151, His154, Glu155, and His158;
supplying the computer modeling application with a set of structure coordinates of a chemical entity; and
determining whether the chemical entity is expected to bind to the molecule or molecular complex, wherein binding to the molecule or molecular complex is indicative of potential modification of S. aureus peptide deformylase activity.

9. A computer-assisted method for identifying a potential modifier of S. aureus peptide deformylase activity comprising:
supplying a computer modeling application with a set of structure coordinates of a molecule or molecular complex, the molecule or molecular complex comprising at least a portion of at least one S. aureus peptide deformylase or S. aureus peptide deformylase-like active site, the active site comprising amino acids Arg56, Ser57, Gly58, Val59, Gly60, Leu61, Ala62, Leu105, Thr107, Gly108, Glu109, Gly110, Cys111, Leu112, Ser113, Ile150, Val151, His154, Glu155, and His158;
supplying the computer modeling application with a set of structure coordinates of a chemical entity; and
determining whether the chemical entity is expected to bind to the molecule or molecular complex, wherein binding to the molecule or molecular complex is indicative of potential modification of S. aureus peptide deformylase activity.

10. A computer-assisted method for designing a potential modifier of S. aureus peptide deformylase activity de novo comprising:
supplying a computer modeling application with a set of structure coordinates of a molecule or molecular complex, the molecule or molecular complex comprising at least a portion of at least one S. aureus peptide deformylase or S. aureus peptide deformylase-like active site, wherein the active site comprises amino acids Arg56, Ser57, Gly58, Val59, Gly60, Leu61, Ala62, Leu105, Thr107, Gly108, Glu109, Gly110, Cys111, Leu112, Ser113, Ile150, Val151, His154, Glu155, and His158;
forming a chemical entity represented by set of structure coordinates; and
determining whether the chemical entity is expected to bind to the molecule or molecular complex, wherein binding to the molecule or molecular complex is indicative of potential modification of S. aureus peptide deformylase activity.


SEQUENCE LISTING (11 sequences)

SEQ ID NO: 1 - protein, 213 aa, Staphylococcus aureus
MYEYLNNLFTVIQLKQIKIRKVQYMLTMKDIIRDGHPTLRQKAAELELPLTKEEKETLIAMREFLVNSQDEEIAKRYGLRSGVGLAAPQINISKRMIAVLIPDDGSGKSYDYMLVNPKIVSHSVQEAYLPTGEGCLSVDDNVAGLVHRHNKITIKAKDIEGNDIQLRLKGYPAIVFQHEIDHLNGVMFYDHIDKDHPLQPHTDAVEVHQHHHH

SEQ ID NO: 2 - protein, 168 aa, Escherichia coli
SVLQVLHIPDERLRKVAKPVEEVNAEIQRIVDDMFETMYAEEGIGLAATQVDIHQRIIVIDVSENRDERLVLINPELLEKSGETGIEEGCLSIPEQRALVPRAEKVKIRALDRDGKPFELEADGLLAICIQHEMDHLVGKLFMDYLSPLKQQRIRQKVEKLDRLKARA

SEQ ID NO: 3 - protein, 169 aa, Haemophilus influenzae
MTALNVLIYPDDHLKVVCEPVTKVNDAIRKIVDDMFDTMYQEKGIGLAAPQVDILQRIITIDVEGDKQNQFVLINPEILASEGETGIEEGCLSIPGFRALVPRKEKVTVRALDRDGKEFTLDADGLLAICIQHEIDHLNGILFVDYLSPLKRQRIKEKLIKYKKQIAKS

SEQ ID NO: 4 - protein, 160 aa, Bacillus subtilis
MAVKKVVTHPAEVLETPAETVTVFDKKLKKLLDDMYDTMLEMDGVGLAAPQIGILKRAAVVEIGDDRGRIDLVNPEILEKSGEQTGIEGCLSFPNVYGDVTRADYVKVRAFNRQGKPFILEARGFLARAVQHEMDHLDGVLFTSKISKYYTEDELADMEG

SEQ ID NO: 5 - protein, 216 aa, Mycoplasma pneumoniae
MTKILPVSTISIFRIILILPQINMELLPTKAWLVLDDVKEINEPTKPVQFPLDQASLDCIAKMMAYVDASYNGDAEKYGIIPGIGIAANQIGYWKQMFYIHLMDGGVEHKCLLINPKIINLSANKSFLKSGEGCLSVPKMHQGYVIRHEWITITGFDWLQQKEITITATGLFGMCLQHEFDHLQGRFYYHRINPLNPLFTNKEWKVINPALPSDSE

SEQ ID NO: 6 - protein, 170 aa, Staphylococcus aureus
MAIKKLVPASHPILTKKAQAVKTFDDSLKRLLQDLEDTMYAQEAAGLCAPQINQSLQVAIIDMEMEGLLQLVNPKIISQSNETITDLEGSITLPDVYGEVTRSKMIVVESYDVNGNKVELTAHEDVARMILHIIDQMNGIPFTERADRILTDKEVEAYFINDRSHHHHHH

SEQ ID NO: 7 - protein, 172 aa, Staphylococcus aureus
MLTMKDIIRDGHLRQKAAEIELPLTEEKETLIMREFLVNSQDEEIAKRYGGVGLAAPQINISKRMIAVLIPDDGSGKSYDLVNPKIVSSVQEAYLPTEGCLVDDNVALVHRHNRIIKAKDIEGNDIQLRLKGYPAIVFQHEIDHINGVMFYDHIDKDHPLQPHTDAVEVHHH

SEQ ID NO: 8 - protein, 138 aa, Escherichia coli
SVLRKVAKPVEEVEIQRIVDMFETMYGIGLAATQVDIHQRIIVIDVSENLINPELLESGETGIEGCLIPEQRLVPRAEKVIRALDRDGKPFELEADGLIAICIQHEMDHLVGKLFMDYLSPLKQQRIRQKVEKLDRLK

SEQ ID NO: 9 - protein, 6 aa, Artificial
  Amino Acid Residue
GXGLAA

SEQ ID NO: 10 - protein, 5 aa, Artificial
  Amino Acid Residue
EGCLS

SEQ ID NO: 11 - protein, 10 aa, Artificial
  Amino Acid Residue
IXXQHEXDHL